SEQUENCE LISTING (261 sequences)

SEQ ID NO: 1 - protein, 14 aa, Human coronavirus
TAYNGYLTSSSKTP

SEQ ID NO: 2 - protein, 15 aa, Human coronavirus
LVPNQPYPNASFDNF

SEQ ID NO: 3 - protein, 14 aa, Human coronavirus
NRFTTTLNDFNLVA

SEQ ID NO: 4 - protein, 18 aa, Human coronavirus
QGLLPPKNSIDAFKLNIK

SEQ ID NO: 5 - protein, 20 aa, Human coronavirus
NTCVGSDNVTDFNAIATCDW

SEQ ID NO: 6 - protein, 15 aa, Human coronavirus
FYSYATHSDKFTDGV

SEQ ID NO: 7 - protein, 19 aa, Human coronavirus
TESNKKFLPFQQFGRDIAD

SEQ ID NO: 8 - protein, 19 aa, Human coronavirus
TDAVRDPQTLEILDITPCS

SEQ ID NO: 9 - protein, 27 aa, Human coronavirus
QVAVLYQDVNCTEVPVAIHADQLTPTW

SEQ ID NO: 10 - protein, 17 aa, Human coronavirus
CASYQTQTNSPRRARSV

SEQ ID NO: 11 - protein, 23 aa, Human coronavirus
ARSVASQSIIAYTMSLGAENSVA

SEQ ID NO: 12 - protein, 21 aa, Human coronavirus
RALTGIAVEQDKNTQEVFAQV

SEQ ID NO: 13 - protein, 21 aa, Human coronavirus
FAQVKQIYKTPPIKDFGGFNF

SEQ ID NO: 14 - protein, 16 aa, Human coronavirus
NFSQILPDPSKPSKRS

SEQ ID NO: 15 - protein, 14 aa, Human coronavirus
KRSFIEDLLFNKVT

SEQ ID NO: 16 - protein, 23 aa, Human coronavirus
PELDSFKEELDKYFKNHTSPDVD

SEQ ID NO: 17 - protein, 17 aa, Human coronavirus
SPDVDLGDISGINASVV

SEQ ID NO: 18 - protein, 23 aa, Human coronavirus
EHDYQIGGYTEKWESGVKDCVVL

SEQ ID NO: 19 - protein, 15 aa, Human coronavirus
CDIKDLPKEITVATS

SEQ ID NO: 20 - protein, 15 aa, Human coronavirus
QRVAGDSGFAAYSRY

SEQ ID NO: 21 - protein, 21 aa, Human coronavirus
GSKSPIQYIDIGNYTVSCLPF

SEQ ID NO: 22 - protein, 17 aa, Human coronavirus
ALLLLDRLNQLESKMSG

SEQ ID NO: 23 - protein, 24 aa, Human coronavirus
NQLESKMSGKGQQQQGQTVTKKSA

SEQ ID NO: 24 - protein, 18 aa, Human coronavirus
QTVTKKSAAEASKKPRQK

SEQ ID NO: 25 - protein, 16 aa, Human coronavirus
RGPEQTQGNFGDQELI

SEQ ID NO: 26 - protein, 17 aa, Human coronavirus
AYKTFPPTEPKKDKKKK

SEQ ID NO: 27 - protein, 16 aa, Human coronavirus
KDKKKKADETQALPQR

SEQ ID NO: 28 - protein, 25 aa, Human coronavirus
ADETQALPQRQKKQQTVTLLPAADL

SEQ ID NO: 29 - protein, 24 aa, Human coronavirus
TLLPAADLDDFSKQLQQSMSSADS

SEQ ID NO: 30 - protein, 14 aa, Human coronavirus
LGDELGTDPYEDFQ

SEQ ID NO: 31 - protein, 15 aa, Human coronavirus
LYIDINGNLHPDSAT

SEQ ID NO: 32 - protein, 15 aa, Human coronavirus
ATKYLVQQESPFVMM

SEQ ID NO: 33 - protein, 14 aa, Human coronavirus
IVFDGKSKCEESSA

SEQ ID NO: 34 - protein, 15 aa, Human coronavirus
LNDFSNSGSDVLYQP

SEQ ID NO: 35 - protein, 14 aa, Human coronavirus
LSFCAFAVDAAKAY

SEQ ID NO: 36 - protein, 14 aa, Human coronavirus
YDFGDFIQTTPGSG

SEQ ID NO: 37 - protein, 14 aa, Human coronavirus
DFIQTTPGSGVPVV

SEQ ID NO: 38 - protein, 15 aa, Human coronavirus
FLLKYNENGTITDAV

SEQ ID NO: 39 - protein, 14 aa, Human coronavirus
GVLTESNKKFLPFQ

SEQ ID NO: 40 - protein, 15 aa, Human coronavirus
YQTQTNSPRRARSVA

SEQ ID NO: 41 - protein, 14 aa, Human coronavirus
NTQEVFAQVKQIYK

SEQ ID NO: 42 - protein, 21 aa, Human coronavirus
MADSNGTITVEELKKLLEQWN

SEQ ID NO: 43 - protein, 17 aa, Human coronavirus
KEPCSSGTYEGNSPFHP

SEQ ID NO: 44 - protein, 14 aa, Human coronavirus
TLLPAADLDDFSKQ

SEQ ID NO: 45 - protein, 14 aa, Human coronavirus
AADLDDFSKQLQQS

SEQ ID NO: 46 - protein, 14 aa, Human coronavirus
YRKVLLRKNGNKGA

SEQ ID NO: 47 - protein, 15 aa, Human coronavirus
LLRKNGNKGAGGHSY

SEQ ID NO: 48 - protein, 19 aa, Human coronavirus
GNKGAGGHSYGADLKSFDL

SEQ ID NO: 49 - protein, 14 aa, Human coronavirus
DELGTDPYEDFQEN

SEQ ID NO: 50 - protein, 14 aa, Human coronavirus
WNTKHSSGVTRELM

SEQ ID NO: 51 - protein, 14 aa, Human coronavirus
GVYCCREHEHEIAW

SEQ ID NO: 52 - protein, 14 aa, Human coronavirus
HEIAWYTERSEKSY

SEQ ID NO: 53 - protein, 14 aa, Human coronavirus
SYELQTPFEIKLAK

SEQ ID NO: 54 - protein, 14 aa, Human coronavirus
AKKFDTFNGECPNF

SEQ ID NO: 55 - protein, 14 aa, Human coronavirus
CDHCGETSWQTGDF

SEQ ID NO: 56 - protein, 17 aa, Human coronavirus
LTNIFGTVYEKLKPVLD

SEQ ID NO: 57 - protein, 14 aa, Human coronavirus
GVEFLRDGWEIVKF

SEQ ID NO: 58 - protein, 20 aa, Human coronavirus
TGDLQPLEQPTSEAVEAPLV

SEQ ID NO: 59 - protein, 18 aa, Human coronavirus
KGGAPTKVTFGDDTVIEV

SEQ ID NO: 60 - protein, 14 aa, Human coronavirus
NITFELDERIDKVL

SEQ ID NO: 61 - protein, 18 aa, Human coronavirus
EFACVVADAVIKTLQPVS

SEQ ID NO: 62 - protein, 16 aa, Human coronavirus
NKGEDIQLLKSAYENF

SEQ ID NO: 63 - protein, 15 aa, Human coronavirus
YDKLVSSFLEMKSEK

SEQ ID NO: 64 - protein, 16 aa, Human coronavirus
AFYILPSIISNEKQEI

SEQ ID NO: 65 - protein, 14 aa, Human coronavirus
LGYVTHGLNLEEAA

SEQ ID NO: 66 - protein, 15 aa, Human coronavirus
THGLNLEEAARYMRS

SEQ ID NO: 67 - protein, 14 aa, Human coronavirus
TAYNGYLTSSSKTP

SEQ ID NO: 68 - protein, 14 aa, Human coronavirus
GEVITFDNLKTLLS

SEQ ID NO: 69 - protein, 18 aa, Human coronavirus
GRYMSALNHTKKWKYPQV

SEQ ID NO: 70 - protein, 15 aa, Human coronavirus
TVGELGDVRETMSYL

SEQ ID NO: 71 - protein, 17 aa, Human coronavirus
MGTLSYEQFKKGVQIPC

SEQ ID NO: 72 - protein, 16 aa, Human coronavirus
FTCASEYTGNYQCGHY

SEQ ID NO: 73 - protein, 14 aa, Human coronavirus
YQCGHYKHITSKET

SEQ ID NO: 74 - protein, 15 aa, Human coronavirus
LVPNQPYPNASFDNF

SEQ ID NO: 75 - protein, 14 aa, Human coronavirus
FPDLNGDVVAIDYK

SEQ ID NO: 76 - protein, 17 aa, Human coronavirus
NSFDVLKSEDAQGMDNL

SEQ ID NO: 77 - protein, 18 aa, Human coronavirus
SEEVVENPTIQKDVLECN

SEQ ID NO: 78 - protein, 14 aa, Human coronavirus
GLAAVNSVPWDTIA

SEQ ID NO: 79 - protein, 18 aa, Human coronavirus
TIANYAKPFLNKVVSTTT

SEQ ID NO: 80 - protein, 16 aa, Human coronavirus
FLNKVVSTTTNIVTRC

SEQ ID NO: 81 - protein, 14 aa, Human coronavirus
VKMFDAYVNTFSST

SEQ ID NO: 82 - protein, 14 aa, Human coronavirus
FSSTFNVPMEKLKT

SEQ ID NO: 83 - protein, 16 aa, Human coronavirus
TLVATAEAELAKNVSL

SEQ ID NO: 84 - protein, 15 aa, Human coronavirus
SAAKKNNLPFKLTCA

SEQ ID NO: 85 - protein, 16 aa, Human coronavirus
VHVMSKHTDFSSEIIG

SEQ ID NO: 86 - protein, 14 aa, Human coronavirus
DFDTWFSQRGGSYT

SEQ ID NO: 87 - protein, 14 aa, Human coronavirus
TFDSEYCRHGTCER

SEQ ID NO: 88 - protein, 16 aa, Human coronavirus
DVLYQPPQTSITSAVL

SEQ ID NO: 89 - protein, 16 aa, Human coronavirus
NRFTTTLNDFNLVAMK

SEQ ID NO: 90 - protein, 14 aa, Human coronavirus
LTQDHVDILGPLSA

SEQ ID NO: 91 - protein, 15 aa, Human coronavirus
GMNGRTILGSALLED

SEQ ID NO: 92 - protein, 18 aa, Human coronavirus
QGLLPPKNSIDAFKLNIK

SEQ ID NO: 93 - protein, 16 aa, Human coronavirus
KLKKSLNVAKSEFDRD

SEQ ID NO: 94 - protein, 15 aa, Human coronavirus
LRANSAVKLQNNELS

SEQ ID NO: 95 - protein, 15 aa, Human coronavirus
AVKLQNNELSPVALR

SEQ ID NO: 96 - protein, 14 aa, Human coronavirus
CAAGTTQTACTDDN

SEQ ID NO: 97 - protein, 15 aa, Human coronavirus
LALLSDLQDLKWARF

SEQ ID NO: 98 - protein, 15 aa, Human coronavirus
ATVRLQAGNATEVPA

SEQ ID NO: 99 - protein, 16 aa, Human coronavirus
VKMLCTHTGTGQAITV

SEQ ID NO: 100 - protein, 14 aa, Human coronavirus
KYVQIPTTCANDPV

SEQ ID NO: 101 - protein, 15 aa, Human coronavirus
RVCGVSAARLTPCGT

SEQ ID NO: 102 - protein, 14 aa, Human coronavirus
AVAKHDFFKFRIDG

SEQ ID NO: 103 - protein, 14 aa, Human coronavirus
GDFIQTTPGSGVPV

SEQ ID NO: 104 - protein, 14 aa, Human coronavirus
QTYHPNCVNCLDDR

SEQ ID NO: 105 - protein, 16 aa, Human coronavirus
ALTNNVAFQTVKPGNF

SEQ ID NO: 106 - protein, 14 aa, Human coronavirus
GFFKEGSSVELKHF

SEQ ID NO: 107 - protein, 20 aa, Human coronavirus
QLLFVVEVVDKYFDCYDGGC

SEQ ID NO: 108 - protein, 19 aa, Human coronavirus
SLYVKPGGTSSGDATTAYA

SEQ ID NO: 109 - protein, 16 aa, Human coronavirus
NRDVDTDFVNEFYAYL

SEQ ID NO: 110 - protein, 14 aa, Human coronavirus
SQGLVASIKNFKSV

SEQ ID NO: 111 - protein, 14 aa, Human coronavirus
QHTMLVKQGDDYVY

SEQ ID NO: 112 - protein, 14 aa, Human coronavirus
VDDIVKTDGTLMIE

SEQ ID NO: 113 - protein, 14 aa, Human coronavirus
AIDAYPLTKHPNQE

SEQ ID NO: 114 - protein, 14 aa, Human coronavirus
GCDVTDVTQLYLGG

SEQ ID NO: 115 - protein, 14 aa, Human coronavirus
GLYKNTCVGSDNVT

SEQ ID NO: 116 - protein, 20 aa, Human coronavirus
NTCVGSDNVTDFNAIATCDW

SEQ ID NO: 117 - protein, 14 aa, Human coronavirus
YQKVGMQKYSTLQG

SEQ ID NO: 118 - protein, 14 aa, Human coronavirus
STLQGPPGTGKSHF

SEQ ID NO: 119 - protein, 15 aa, Human coronavirus
ARARVECFDKFKVNS

SEQ ID NO: 120 - protein, 14 aa, Human coronavirus
DPAQLPAPRTLLTK

SEQ ID NO: 121 - protein, 14 aa, Human coronavirus
PRTLLTKGTLEPEY

SEQ ID NO: 122 - protein, 14 aa, Human coronavirus
IFTQTTETAHSCNV

SEQ ID NO: 123 - protein, 14 aa, Human coronavirus
GCHATREAVGTNLP

SEQ ID NO: 124 - protein, 14 aa, Human coronavirus
VQMLSDTLKNLSDR

SEQ ID NO: 125 - protein, 16 aa, Human coronavirus
FDYVYNPFMIDVQQWG

SEQ ID NO: 126 - protein, 14 aa, Human coronavirus
MIDVQQWGFTGNLQ

SEQ ID NO: 127 - protein, 15 aa, Human coronavirus
TGNLQSNHDLYCQVH

SEQ ID NO: 128 - protein, 21 aa, Human coronavirus
LADKFPVLHDIGNPKAIKCVP

SEQ ID NO: 129 - protein, 15 aa, Human coronavirus
FYSYATHSDKFTDGV

SEQ ID NO: 130 - protein, 15 aa, Human coronavirus
FDTRVLSNLNLPGCD

SEQ ID NO: 131 - protein, 16 aa, Human coronavirus
QVVSDIDYVPLKSATC

SEQ ID NO: 132 - protein, 15 aa, Human coronavirus
PSVGPKQASLNGVTL

SEQ ID NO: 133 - protein, 16 aa, Human coronavirus
GVVQQLPETYFTQSRN

SEQ ID NO: 134 - protein, 14 aa, Human coronavirus
AMDEFIERYKLEGY

SEQ ID NO: 135 - protein, 14 aa, Human coronavirus
LAKRFKESPFELED

SEQ ID NO: 136 - protein, 18 aa, Human coronavirus
FITDAQTGSSKCVCSVID

SEQ ID NO: 137 - protein, 15 aa, Human coronavirus
IKSQDLSVVSKVVKV

SEQ ID NO: 138 - protein, 14 aa, Human coronavirus
YPKLQSSQAWQPGV

SEQ ID NO: 139 - protein, 14 aa, Human coronavirus
QRMLLEKCDLQNYG

SEQ ID NO: 140 - protein, 15 aa, Human coronavirus
HFGAGSDKGVAPGTA

SEQ ID NO: 141 - protein, 14 aa, Human coronavirus
ITEHSWNADLYKLM

SEQ ID NO: 142 - protein, 16 aa, Human coronavirus
LSSYSLFDMSKFPLKL

SEQ ID NO: 143 - protein, 14 aa, Human coronavirus
SLLLCRMNSRNYIA

SEQ ID NO: 144 - protein, 14 aa, Human coronavirus
QCVNLTTRTQLPPA

SEQ ID NO: 145 - protein, 19 aa, Human coronavirus
TQLPPAYTNSFTRGVYYPD

SEQ ID NO: 146 - protein, 15 aa, Human coronavirus
FHAIHVSGTNGTKRF

SEQ ID NO: 147 - protein, 17 aa, Human coronavirus
NLVRDLPQGFSALEPLV

SEQ ID NO: 148 - protein, 18 aa, Human coronavirus
LALHRSYLTPGDSSSGWT

SEQ ID NO: 149 - protein, 20 aa, Human coronavirus
KYNENGTITDAVDCALDPLS

SEQ ID NO: 150 - protein, 18 aa, Human coronavirus
IYQAGSTPCNGVEGFNCY

SEQ ID NO: 151 - protein, 14 aa, Human coronavirus
VLSFELLHAPATVC

SEQ ID NO: 152 - protein, 15 aa, Human coronavirus
LLHAPATVCGPKKST

SEQ ID NO: 153 - protein, 14 aa, Human coronavirus
VKNKCVNFNFNGLT

SEQ ID NO: 154 - protein, 19 aa, Human coronavirus
TESNKKFLPFQQFGRDIAD

SEQ ID NO: 155 - protein, 19 aa, Human coronavirus
TDAVRDPQTLEILDITPCS

SEQ ID NO: 156 - protein, 27 aa, Human coronavirus
QVAVLYQDVNCTEVPVAIHADQLTPTW

SEQ ID NO: 157 - protein, 18 aa, Human coronavirus
IHADQLTPTWRVYSTGSN

SEQ ID NO: 158 - protein, 15 aa, Human coronavirus
YSTGSNVFQTRAGCL

SEQ ID NO: 159 - protein, 17 aa, Human coronavirus
CASYQTQTNSPRRARSV

SEQ ID NO: 160 - protein, 23 aa, Human coronavirus
ARSVASQSIIAYTMSLGAENSVA

SEQ ID NO: 161 - protein, 14 aa, Human coronavirus
PVSMTKTSVDCTMY

SEQ ID NO: 162 - protein, 21 aa, Human coronavirus
RALTGIAVEQDKNTQEVFAQV

SEQ ID NO: 163 - protein, 21 aa, Human coronavirus
FAQVKQIYKTPPIKDFGGFNF

SEQ ID NO: 164 - protein, 16 aa, Human coronavirus
NFSQILPDPSKPSKRS

SEQ ID NO: 165 - protein, 14 aa, Human coronavirus
KRSFIEDLLFNKVT

SEQ ID NO: 166 - protein, 15 aa, Human coronavirus
EDLLFNKVTLADAGF

SEQ ID NO: 167 - protein, 15 aa, Human coronavirus
YGDCLGDIAARDLIC

SEQ ID NO: 168 - protein, 16 aa, Human coronavirus
ICAQKFNGLTVLPPLL

SEQ ID NO: 169 - protein, 15 aa, Human coronavirus
FGAGAALQIPFAMQM

SEQ ID NO: 170 - protein, 15 aa, Human coronavirus
NGIGVTQNVLYENQK

SEQ ID NO: 171 - protein, 14 aa, Human coronavirus
IGKIQDSLSSTASA

SEQ ID NO: 172 - protein, 15 aa, Human coronavirus
VLNDILSRLDKVEAE

SEQ ID NO: 173 - protein, 14 aa, Human coronavirus
LSRLDKVEAEVQID

SEQ ID NO: 174 - protein, 16 aa, Human coronavirus
AICHDGKAHFPREGVF

SEQ ID NO: 175 - protein, 17 aa, Human coronavirus
HWFVTQRNFYEPQIITT

SEQ ID NO: 176 - protein, 17 aa, Human coronavirus
VIGIVNNTVYDPLQPEL

SEQ ID NO: 177 - protein, 14 aa, Human coronavirus
TVYDPLQPELDSFK

SEQ ID NO: 178 - protein, 23 aa, Human coronavirus
PELDSFKEELDKYFKNHTSPDVD

SEQ ID NO: 179 - protein, 17 aa, Human coronavirus
SPDVDLGDISGINASVV

SEQ ID NO: 180 - protein, 16 aa, Human coronavirus
EIDRLNEVAKNLNESL

SEQ ID NO: 181 - protein, 14 aa, Human coronavirus
EVAKNLNESLIDLQ

SEQ ID NO: 182 - protein, 14 aa, Human coronavirus
SCGSCCKFDEDDSE

SEQ ID NO: 183 - protein, 16 aa, Human coronavirus
FDEDDSEPVLKGVKLH

SEQ ID NO: 184 - protein, 16 aa, Human coronavirus
TIGTVTLKQGEIKDAT

SEQ ID NO: 185 - protein, 23 aa, Human coronavirus
EHDYQIGGYTEKWESGVKDCVVL

SEQ ID NO: 186 - protein, 14 aa, Human coronavirus
MADSNGTITVEELK

SEQ ID NO: 187 - protein, 16 aa, Human coronavirus
RSMWSFNPETNILLNV

SEQ ID NO: 188 - protein, 15 aa, Human coronavirus
CDIKDLPKEITVATS

SEQ ID NO: 189 - protein, 15 aa, Human coronavirus
KLGASQRVAGDSGFA

SEQ ID NO: 190 - protein, 15 aa, Human coronavirus
QRVAGDSGFAAYSRY

SEQ ID NO: 191 - protein, 15 aa, Human coronavirus
SSGTYEGNSPFHPLA

SEQ ID NO: 192 - protein, 19 aa, Human coronavirus
LIELCVDEAGSKSPIQYID

SEQ ID NO: 193 - protein, 21 aa, Human coronavirus
GSKSPIQYIDIGNYTVSCLPF

SEQ ID NO: 194 - protein, 14 aa, Human coronavirus
RSKQRRPQGLPNNT

SEQ ID NO: 195 - protein, 14 aa, Human coronavirus
DLKFPRGQGVPINT

SEQ ID NO: 196 - protein, 15 aa, Human coronavirus
NTNSSPDDQIGYYRR

SEQ ID NO: 197 - protein, 14 aa, Human coronavirus
KDHIGTRNPANNAA

SEQ ID NO: 198 - protein, 15 aa, Human coronavirus
RGTSPARMAGNGGDA

SEQ ID NO: 199 - protein, 17 aa, Human coronavirus
MAGNGGDAALALLLLDR

SEQ ID NO: 200 - protein, 17 aa, Human coronavirus
ALLLLDRLNQLESKMSG

SEQ ID NO: 201 - protein, 24 aa, Human coronavirus
NQLESKMSGKGQQQQGQTVTKKSA

SEQ ID NO: 202 - protein, 18 aa, Human coronavirus
QTVTKKSAAEASKKPRQK

SEQ ID NO: 203 - protein, 16 aa, Human coronavirus
RGPEQTQGNFGDQELI

SEQ ID NO: 204 - protein, 16 aa, Human coronavirus
IKLDDKDPNFKDQVIL

SEQ ID NO: 205 - protein, 17 aa, Human coronavirus
AYKTFPPTEPKKDKKKK

SEQ ID NO: 206 - protein, 16 aa, Human coronavirus
KDKKKKADETQALPQR

SEQ ID NO: 207 - protein, 25 aa, Human coronavirus
ADETQALPQRQKKQQTVTLLPAADL

SEQ ID NO: 208 - protein, 24 aa, Human coronavirus
TLLPAADLDDFSKQLQQSMSSADS

SEQ ID NO: 209 - protein, 17 aa, Human coronavirus
MVNPSKGSSGLLITKLT

SEQ ID NO: 210 - protein, 18 aa, Human coronavirus
VKQLPEQFDSLNLSAGTQ

SEQ ID NO: 211 - protein, 16 aa, Human coronavirus
SLLATLDDPYVEDSVA

SEQ ID NO: 212 - protein, 15 aa, Human coronavirus
MNKSFLPQFTSDQAV

SEQ ID NO: 213 - protein, 17 aa, Human coronavirus
WLNNFNVPSPLNWERKI

SEQ ID NO: 214 - protein, 16 aa, Human coronavirus
ISASYRFVTFEPFNVS

SEQ ID NO: 215 - protein, 16 aa, Human coronavirus
SRSFFEDLLFDKVKLS

SEQ ID NO: 216 - protein, 19 aa, Human coronavirus
VDKQVILVSNNKEDFDIIQ

SEQ ID NO: 217 - protein, 16 aa, Human coronavirus
LLHSKVKVVPNLYVVV

SEQ ID NO: 218 - protein, 15 aa, Human coronavirus
FDLVQYDFTDYKLEL

SEQ ID NO: 219 - protein, 16 aa, Human coronavirus
PASQVIVNNYDKSAGY

SEQ ID NO: 220 - protein, 15 aa, Human coronavirus
CTGSPYIDDFNKIAS

SEQ ID NO: 221 - protein, 19 aa, Human coronavirus
CWETGKVKPPLNKNYVFTG

SEQ ID NO: 222 - protein, 20 aa, Human coronavirus
AKVRVDCYDKFKINDTTCKY

SEQ ID NO: 223 - protein, 18 aa, Human coronavirus
ISPYNSQNYVAKRVLGVQ

SEQ ID NO: 224 - protein, 14 aa, Human coronavirus
EFRDYFNNNTDSIV

SEQ ID NO: 225 - protein, 18 aa, Human coronavirus
RKASGLFDAIWDAFVAAI

SEQ ID NO: 226 - protein, 16 aa, Human coronavirus
NVGEPIISQPSKLLNG

SEQ ID NO: 227 - protein, 18 aa, Human coronavirus
ISYLNRARGSSAARLEPC

SEQ ID NO: 228 - protein, 26 aa, Human coronavirus
CIKACEVVVTNLNKSAGWPLNKFGKA

SEQ ID NO: 229 - protein, 15 aa, Human coronavirus
VFINKYGLEDFNFEH

SEQ ID NO: 230 - protein, 19 aa, Human coronavirus
TVLNASIPESKPLADDDSA

SEQ ID NO: 231 - protein, 17 aa, Human coronavirus
ASQEGVLSEREKLLHLI

SEQ ID NO: 232 - protein, 19 aa, Human coronavirus
QFGLQDGFYSANFLDDNVL

SEQ ID NO: 233 - protein, 19 aa, Human coronavirus
CDCGSTKLPYYEFEKVHVQ

SEQ ID NO: 234 - protein, 15 aa, Human coronavirus
FDLVQYDFTDYKLEL

SEQ ID NO: 235 - protein, 16 aa, Human coronavirus
SQVIVNNYDKSAGYPF

SEQ ID NO: 236 - protein, 19 aa, Human coronavirus
LSTCDGQKFTDESFYKNMY

SEQ ID NO: 237 - protein, 16 aa, Human coronavirus
FPLKLAGTAVINLRAD

SEQ ID NO: 238 - protein, 15 aa, Human coronavirus
HATRDSIGTNFPLQL

SEQ ID NO: 239 - protein, 15 aa, Human coronavirus
CTGSPYIDDFNRIAS

SEQ ID NO: 240 - protein, 21 aa, Human coronavirus
AKVRVECYDKFKINDTTRKYV

SEQ ID NO: 241 - protein, 18 aa, Human coronavirus
ASSRSAIEDLLFDKVKLS

SEQ ID NO: 242 - protein, 16 aa, Human coronavirus
SQVIVNNYDKSAGYPF

SEQ ID NO: 243 - protein, 19 aa, Human coronavirus
LSTCDGQKFTDESFYKNMY

SEQ ID NO: 244 - protein, 16 aa, Human coronavirus
AETQKATEEAFKQSYA

SEQ ID NO: 245 - protein, 19 aa, Human coronavirus
SWEIGKVKPPLNKNYVFTG

SEQ ID NO: 246 - protein, 21 aa, Human coronavirus
AKVRVECYDKFKINDTTRKYV

SEQ ID NO: 247 - protein, 15 aa, Human coronavirus
HATRDSIGTNFPLQL

SEQ ID NO: 248 - protein, 19 aa, Human coronavirus
DVIDNEIIVKPNISLCVPL

SEQ ID NO: 249 - protein, 18 aa, Human coronavirus
KHSDFGLGDLVDSYFEND

SEQ ID NO: 250 - protein, 16 aa, Human coronavirus
CITSREVVVTNLNKSA

SEQ ID NO: 251 - protein, 19 aa, Human coronavirus
DVIDNEIIVKPNISLCVPL

SEQ ID NO: 252 - protein, 18 aa, Human coronavirus
KHSDFGLGDLVDSYFEND

SEQ ID NO: 253 - protein, 30 aa, Human coronavirus
LYEQVVMDYLDNLKPRVEAPKQEEPPNTED

SEQ ID NO: 254 - protein, 27 aa, Human coronavirus
VSVSSPDAVTTYNGYLTSSSKTSEEHF

SEQ ID NO: 255 - protein, 27 aa, Human coronavirus
LGDELGTDPIEDYEQNWNTKHGSGALR

SEQ ID NO: 256 - protein, 26 aa, Human coronavirus
FTLRSITAQPVKIDNASPASTVHATA

SEQ ID NO: 257 - protein, 28 aa, Human coronavirus
TIVVTEGDGISTPKLKEDYQIGGYSEDR

SEQ ID NO: 258 - protein, 24 aa, Human coronavirus
SSGVANPAMDPIYDEPTTTTSVPL

SEQ ID NO: 259 - protein, 24 aa, Human coronavirus
MADNGTITVEELKQLLEQWNLVIG

SEQ ID NO: 260 - protein, 40 aa, Human coronavirus
TPKDHIGTRNPNNNAATVLQLPQGTTLPKGFYAEGSRGGS

SEQ ID NO: 261 - protein, 20 aa, Human coronavirus
FSLDVSEKSGNFKHLREFVF

(19) United States
(12) Patent Application Publication
(10) Pub. No.: US 2023/0184764 A1
(43) Pub. Date: Jun. 15, 2023

(54) PEPTIDE SEQUENCES FOR DETECTION AND DIFFERENTIATION OF ANTIBODY RESPONSES TO SARS-COV-2 AND OTHER HUMAN CORONA VIRUSES

(71) Applicant: The Trustees of Columbia University in the City of New York, New York, NY (US)

(72) Inventors: Walter Ian Lipkin, New York, NY (US); Nischay MISHRA, New York, NY (US); Thomas BRIESE, White Plains, NY (US); Cheng GUO, Fort Lee, NJ (US); Shreyas JOSHI, Fort Lee, NJ (US); Rafal TOKARZ, Queens Village, NY (US)

(21) Appl. No.: 17/917,184

(22) PCT Filed: Apr. 6, 2021
(86) PCT No.: PCT/US2021/025976

§ 371 Date: Oct. 5, 2022

Related U.S. Application Data
(60) Provisional application No. 63/064,074, filed on Aug. 11, 2020.
(60) Provisional application No. 63/005,865, filed on Apr. 6, 2020.

(51) Int. Cl. G01N 33/569

(52) U.S. Cl. CPC G01N 33/56983; G01N 2333/165; G01N 2469/20

(57) ABSTRACT

The current disclosure provides compositions, methods, and kits for detecting the exposure to and infection by certain viruses. Specifically, the current disclosure allows for the rapid differential serological detection of exposure to, and infection by viruses. In particular, the current disclosure allows for the rapid differential serological detection of exposure to, and infection by corona virus (SARS-CoV-2). The current disclosure provides for peptides for the differential detection of SARS-CoV-2.

### CROSS-REFERENCE TO OTHER APPLICATIONS

The present application claims priority to U.S. patent applications Ser. Nos. 63/005,865 filed Apr. 6, 2020 and 63/064,074 filed Aug. 11, 2020, both of which are hereby incorporated by reference in its entirety.

### STATEMENT OF GOVERNMENT SUPPORT

This invention was made with government support under AI109761 awarded by the National Institutes of Health. As such, the United States government has certain rights in this invention.

### FIELD

This disclosure herein relates to the field of detection of antibody responses and exposure to viruses, in particular the detection of an antibody response and exposure to corona virus (SARS-CoV-2).

### BACKGROUND

In December 2019, a cluster of cases were found positive for severe pneumonia-like illness in Wuhan, Hubei Province, China. A cluster of 41 patients that developed severe pneumonia like illness were shown to be infected with a novel coronavirus (nCoV) (Coronaviridae Study Group 2020). Sequence analyses of nCoV indicated it was a beta coronavirus, with high similarity to severe acute respiratory syndrome coronavirus (SARS-CoV). Subsequently, the International Committee on Taxonomy of Viruses (ICTV)/WHO named this virus severe acute respiratory syndrome coronavirus 2 (SARS-CoV-2) and its disease coronavirus disease 2019 (COVID-19) (Zhou et al. 2020). On Jan. 30, 2020, the WHO declared COVID-19 a global health emergency. Within 12 weeks after identification of first cluster, there were greater than 790,000 positive cases of COVID-19 globally, with 38,000 fatalities (19%). A COVID-19 outbreak began in the United States with an evacuated patient from Wuhan on Jan. 15, 2020. On Feb. 4, 2020, the Secretary of the Department of Health and Human Services (HHS), USA also declared a public health emergency with significant potential to affect national security or the health and security of United States citizens living abroad. As of April 2020, the virus had resulted in over a million cases worldwide, with tens of thousand deaths.

The severity and rapid progression of COVID-19 has impacted the majority of the population through physical, social, and financial distress. The mitigation and containment measures forced global lockdowns, curfews and home isolations, that have already affected global economy to a greater extent than the terrorist attacks of Sep. 11, 2001, or the 2008 financial crisis. It has been estimated the pandemic may cost approximately $3 trillion and result in a loss of 5-10% global GDP

Diagnostic tests are critical for clinical and public health management of COVID-19. These tests fall into two broad categories: molecular assays used to identify infected individuals and to detect environmental contamination; and serological assays used to determine who has been exposed.

Molecular assays for detection of SARS-CoV-2 are useful in diagnosis of active infection but cannot provide insight into historical infection, especially in those individuals with mild or non-existence symptoms.

Additionally, vaccines are at the forefront of COVID-19 research. As these vaccines are developed, there is a need for rapid reliable assays for testing the efficacy of such vaccines.

Thus, there is a need for a sensitive, specific, and inexpensive high throughput serological assay for the diagnosis of SARS-CoV-2 exposure.

### SUMMARY

The current disclosure provides compositions, methods, devices, and kits for detecting the exposure to, and infection by, certain viruses. Specifically, the current disclosure allows for the rapid differential serological detection of exposure to, and infection by viruses. In particular, the current disclosure allows for the rapid serological differential detection of exposure to, and infection by corona virus (SARS-CoV-2), and discrimination of antibody responses to linear epitopes specific to seven human coronaviruses: SARS-CoV-2; SARS; MERS; NL-63; OC-43; 229E; and HKU1.

Described herein is the identification of peptides that will enable specific and sensitive serological differential diagnosis of coronavirus infections on a wide range of platforms including but not limited to microarrays, ELISA, RIA, lateral flow, western blot, and bead-based assays. These assays will allow identification of individuals who are immune to future infection, capable of deployment to the workforce and who may serve as donors of antibodies for control of COVID-19. Cross-reactivity between SARS-CoV-2, SARS-CoV, MERS-CoV and seasonal coronaviruses (e.g., OC43, 229E, HKU1, and NL63) complicate differential serodiagnosis and efforts to investigate the epidemiology of infection and linkage to disease. Serology by plaque reduction neutralization test (PRNT) is an option but PRNTs are expensive, labor-intensive and require live virus and BSL3 facility. Furthermore, antibodies to conserved coronaviruses domains may confound assay specificity, particularly in the acute and early convalescent phase.

Described herein is a sensitive, highly multiplexed, microarray-based assay that enabled the discrimination of antibody responses to linear epitopes specific to seven human coronaviruses: SARS-CoV-2: SARS: MERS: NL-63: OC-43: 229E and HKU1. Also disclosed herein is the identification of 29 highly specific IgG epitopes for SARS-CoV-2 (Table 2) as well as additional 163 IgG epitopes for SARS-CoV-2 (Table 4) and 16 highly specific IgM epitopes for SARS-CoV-2 (Table 3). Also disclosed herein are highly specific epitopes for a human coronavirus other than SARS-CoV-2, listed in Table 5.

The compositions, methods, devices, and kits for detection of exposure to, and infection by SARS-CoV-2 comprise specific peptides, isolated and non-isolated, which are strongly reactive with, and specific for SARS-CoV-2, i.e., reactive and specific epitopes of antibodies to SARS-CoV-2. Additionally, the compositions, methods, devices, and kits for differential detection of exposure to, and infection by SARS-CoV-2 comprise specific peptides, isolated and non-isolated, which are strongly reactive with, and specific for SARS-CoV-2, i.e., reactive and specific epitopes of antibodies to SARS-CoV-2, as well as specific peptides, isolated and non-isolated, which are strongly reactive with, and specific for a human coronavirus other than SARS-CoV-2.

Thus, one embodiment of the present disclosure is a peptide, which is reactive with, and specific for SARS-CoV-2 antibodies, listed in Table 2 (SEQ ID NOs: 1-26), Table 3 (SEQ ID NOs: 30-45), and Table 4 (SEQ ID NOs: 46-208). A further embodiment is a peptide, which is reactive with, and specific for a human coronavirus other than SARS-CoV-2, listed in Table 5 (SEQ ID NOs: 209-261).

A further embodiment is a collection or set of peptides comprising at least one peptide which is reactive with, and specific for SARS-CoV-2 antibodies, listed in Table 2 (SEQ ID NOs: 1-26), Table 3 (SEQ ID NOs: 30-45), and Table 4 (SEQ ID NOs: 46-208). A further embodiment is a collection or set of peptides comprising at least one peptide which is reactive with, and specific for a human coronavirus other than SARS-CoV-2, listed in Table 5 (SEQ ID NOs: 209-261).

Yet a further embodiment of the present disclosure are collections or sets of peptides comprising amino acid sequences shifted one residue across one or more peptides chosen from the group consisting of the peptides that are reactive with, and specific for SARS-CoV-2 antibodies listed in listed in Table 2 (SEQ ID NOs: 1-26), Table 3 (SEQ ID NOs: 30-45), and Table 4 (SEQ ID NOs: 46-208). Yet a further embodiment of the present disclosure are collections or sets of peptides comprising amino acid sequences shifted one residue across one or more peptides chosen from the group consisting of the peptides which is reactive with, and specific for a human coronavirus other than SARS-CoV-2, listed in Table 5 (SEQ ID NOs: 209-261).

These various peptides, or collections or sets of peptides can comprise or consist of peptides, that comprise or consist of 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, up to 25 amino acids in length, up to 30 amino acids in length, up to 35 amino acids in length, up to 40 amino acids in length, and up to 50 amino acids in length.

The number of peptides in a used in a collection or set can range from 2 peptides to a number in the thousands to tens of thousands to hundreds of thousands.

In another aspect, the disclosure provides compositions comprising two or more disclosed peptides.

In another aspect, the disclosure provides nucleic acids comprising a sequence encoding a disclosed peptide. In addition, the disclosure provides vectors comprising such nucleic acids, and host cells comprising such vectors. In certain embodiments, the vector is a shuttle vector. In other embodiments, the vector is an expression vector (e.g., a bacterial or eukaryotic expression vector). In certain embodiments, the host cell is a bacterial cell. In other embodiments, the host cell is a eukaryotic cell.

In non-limiting examples, the antibodies to SARS-CoV-2 and/or other human coronaviruses can be detected using any number of immunodetection techniques, which include but are not necessarily limited to microarrays, ELISA, RIA, lateral flow, western blot, bead-based assays, dipstick type of assay or a SNAP test, multiplex antibody detection techniques of various kinds, or any modification of such assays that are suitable for detecting antibodies of interest.

In one embodiment, the immunodetection technique is in the form of a programmable peptide array.

In certain embodiments, peptides are attached to or immobilized on a solid support. In one embodiment, the peptides are attached to a solid support through a metallic nanolayer. In certain embodiments, the solid support is a bead (e.g., a colloidal particle, metallic nanoparticle or nanoshell, or latex bead), a flow path in a lateral flow immunoassay device (e.g., a porous membrane), a blot (e.g., Western blot, a slot blot, or dot blot), a flow path in an analytical or centrifugal rotor, or a tube or well (e.g., in a plate suitable for an ELISA assay or microarray). In certain embodiments, peptides are isolated (e.g., synthetic and/or purified) peptides. In certain embodiments, peptides are conjugated to a ligand. For example, in certain embodiments, the peptides are biotinylated. In other embodiments, the peptides are conjugated to streptavidin, avidin, or neutravidin. In other embodiments, the peptides are conjugated to a carrier protein (e.g., serum albumin, keyhole limpet hemocyanin (KLH), or an immunoglobulin Fc domain).

In another aspect, the disclosure provides devices. In certain embodiments, the devices are useful for performing an immunoassay. For example, in certain embodiments, the device is a lateral flow immunoassay device. In other embodiments, the device is an analytical or centrifugal rotor. In other embodiments, the device is a tube or a well, e.g., in a plate suitable for an ELISA assay or a microarray. In still other embodiments, the device is an electrochemical, optical, or opto-electronic sensor.

In certain embodiments, the device comprises at least one disclosed peptide. In other embodiments, the device comprises a collection or set of the disclosed peptides as described herein. In certain embodiments, the peptides are attached to or immobilized upon the device.

In another aspect, the disclosure provides methods of detecting in a sample an antibody to an epitope of SARS-CoV-2. In certain embodiments, the methods comprise contacting a sample with one or more disclosed peptides and detecting formation of an antibody-peptide complex comprising said peptide, wherein formation of said complex is indicative of the presence of an antibody to an epitope of SARS-CoV-2 antigen in said sample. In certain embodiments, the methods comprise contacting the sample with a collection or set of different peptides described herein. In certain embodiments, the methods comprise contacting the sample with a collection or set of all of the peptides described herein.

In one embodiment, the disclosure provides a method for the serological detection of exposure to and/or infection by SARS-CoV-2, comprising the use of a peptide or peptides which are reactive with, and specific for SARS-CoV-2 antibodies comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof.

In further embodiments, the method for the serological detection of exposure to and/or infection by SARS-CoV-2 in a sample, comprises: contacting the sample with a peptide or peptides which are reactive with, and specific for SARS-CoV-2 antibodies comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof; and detecting the binding between the anti-SARS-CoV-2 antibodies in the sample and the peptide or peptides.

Yet a further embodiment is a method for the differential serological detection of exposure to and/or infection by SARS-CoV-2, comprising the use of a peptide or peptides which are reactive with, and specific for SARS-CoV-2 antibodies and/or other human coronaviruses comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptides chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof.

In further embodiments, the method for the differential serological detection of exposure to and/or infection by SARS-CoV-2 in a sample, comprises: contacting the sample with a peptide or peptides which are reactive with, and specific for SARS-CoV-2 antibodies and/or other human coronaviruses comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptides chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof; and detecting the binding between the anti-SARS-CoV-2 and/or anti-human coronavirus antibodies in the sample and the peptide or peptides.

In certain embodiments, the peptide or each peptide in the collection or set is an isolated (e.g., synthetic and/or purified) peptide. In certain embodiments, the peptide, peptides or collection or set of peptides is attached to or immobilized upon a solid support. In one embodiment, the peptide, peptides or collection or set of peptides is attached to the solid support through a metallic (e.g., gold) nanolayer. In certain embodiments, the solid support is a bead or plurality of beads (e.g., a colloidal particle, a metallic nanoparticle or nanoshell, or a latex bead), a flow path in a lateral flow immunoassay device (e.g., a porous membrane), a flow path in an analytical or centrifugal rotor, a blot (e.g., Western blot, a slot blot, or dot blot), or a tube or a well (e.g., in a plate suitable for an ELISA assay or microarray). In certain embodiments, the solid support comprises metal, glass, a cellulose-based material (e.g., nitrocellulose), or a polymer (e.g., polystyrene, polyethylene, polypropylene, polyester, nylon, or polysulfone).

A further embodiment is a method for the serological detection of exposure to and/or infection by SARS-CoV-2, comprising the use of a peptide microarray comprising peptide or peptides which are reactive with, and specific for SARS-CoV-2 antibodies comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof.

In further embodiments, the method for the serological detection of exposure to and/or infection by SARS-CoV-2 in a sample, comprises: contacting the sample with a peptide microarray comprising a peptide or peptides which are reactive with, and specific for SARS-CoV-2 antibodies comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof; and detecting the binding between the anti-SARS-CoV-2 antibodies in the sample and the peptide or peptides in the microarray.

Yet a further embodiment is a method for the differential serological detection of exposure to and/or infection by SARS-CoV-2, comprising the use of a peptide microarray comprising a peptide or peptides which are reactive with, and specific for SARS-CoV-2 antibodies and/or other human coronaviruses comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptides chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof.

In further embodiments, the method for the differential serological detection of exposure to and/or infection by SARS-CoV-2 in a sample, comprises: contacting the sample with a peptide microarray comprising a peptide or peptides which are reactive with, and specific for SARS-CoV-2 antibodies and/or other human coronaviruses comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptides chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof; and detecting the binding between the anti-SARS-CoV-2 and/or anti-human coronavirus antibodies in the sample and the peptide or peptides in the microarray.

In certain embodiments, the detecting step comprises performing an ELISA assay. In other embodiments, the detecting step comprises performing a lateral flow immunoassay. In other embodiments, the detecting step comprises performing an agglutination assay. In other embodiments, the detecting step comprises spinning the sample in an analytical or centrifugal rotor. In other embodiments, the detecting step comprises analyzing the sample using a Western blot, a slot blot, or a dot blot. In still other embodiments, the detecting step comprises analyzing the sample with an electrochemical sensor, an optical sensor, or an opto-electronic sensor. In certain embodiments, the detecting step comprises performing a wavelength shift assay.

In some embodiments, the peptides consist of the amino acid sequences SEQ ID NOs: 1-261.

The present disclosure also includes systems and kits for the differential serological detection of exposure to and/or infection by SARS-COV-2.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 shows the multidimensional scaling (MDS) of differential IgM peptide signals in assays of sera from subjects with a history of infection with SARS-CoV-2 (n=100) or without historical exposure to SARS-CoV-2 (controls) (n=32). Based on MDS analysis, samples with exposure of SARS-CoV-2 samples (green triangles) versus controls (red circles) clustered in overlapping groups.

FIG. 2 shows the multidimensional scaling (MDS) of differential IgG peptide signals in assays of sera from subjects with a history of infection with SARS-CoV-2 (n=100) or without historical exposure to SARS-CoV-2 (controls) (n=32). Based on MDS analysis, samples with exposure of SARS-CoV-2 samples (green triangles) versus controls (red circles) clustered into two separate groups.

FIG. 3 is a heatmap showing the proteome-wide linear epitope mapping of SARS-CoV-2-specific IgG antibodies by a HCoV peptide array. X axis represents 981 peptides from SARS-CoV-2 proteins, Y axis represents 132 samples tested using the HCoV peptide array. The heatmap is plotted with normalized values of individual peptide intensity in AU for each of the 132 plasma samples. Panel grids outlined in dotted lines show highly reactive areas in S and N proteins. *ORF1ab protein is large so divided partly in lower panel owing to larger size.

FIG. 4 are graphs of the reactivity intensities of each peptide from 29 immunogenic linear IgG epitopes are plotted on log scale (y-axis). Reactivity to selected peptides is plotted for corresponding groups shown in different colors (x-axis).

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “sample” means any substance containing or presumed to contain antibodies, in particular those to SARS-CoV-2. The sample can be of natural or synthetic origin and can be obtained by any means known to those of skill in the art. The sample can be a sample of tissue or fluid isolated from a subject including but not limited to, plasma, serum, whole blood, spinal fluid, semen, amniotic fluid, lymph fluid, synovial fluid, urine, tears, blood cells, organs, and tissue. Samples can be research, clinical, or environmental. Sample can also be blood products used to transfuse or treat. Samples can also be synthetic and include but are not limited to in vitro cell culture constituents including but not limited to conditioned medium, recombinant cells, and cell components.

As used herein, the term “subject” means any organism including, without limitation, a mammal such as a mouse, a rat, a dog, a guinea pig, a ferret, a rabbit and a primate. In the preferred embodiment, the subject is a human being, a pet or livestock animal.

The term “patient” as used in this application means a human subject.

The term “detection”, “detect”, “detecting” and the like as used herein means as used herein means to discover the presence or existence of.

The terms “identification”, “identify”, “identifying” and the like as used herein means to recognize exposure to a specific virus or viruses in sample from a subject.

The term “peptide” includes any sequence of two or more amino acids. Peptide sequences specifically recited herein are written with the amino terminus on the left and the carboxy terminus on the right.

The term “amino acid,” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate, hippuric acid, octahydroindole-2-carboxylic acid, statine, 1,2,3,4,-tetrahydroisoquinoline-3-carboxylic acid, penicillamine, omithine, citruline, alpha-methylalanine, para-benzoylphenylalanine, phenylglycine, propargylglycine, sarcosine, and tert-butylglycine). The term also includes natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide).

An “antigen” (from antibody-generating) or “immunogen” is a substance that prompts the generation of antibodies and can cause an immune response. They may also be used for diagnostic or patient selection or characterization purposes.

Antibodies (also known as immunoglobulins (Ig)) are globulin proteins that are found in blood or other bodily fluids of vertebrates and are used by the immune system to identify and neutralize foreign objects, such as bacteria and viruses. They are typically made of basic structural units—each with two large heavy chains and two small light chains—to form, for example, monomers with one unit, dimers with two units or pentamers with five units. Antibodies are produced by B cells. There are several different types of antibody heavy chains, and several different kinds of antibodies, which are grouped into different isotypes based on which heavy chain they possess. Five different antibody isotypes are known in mammals, which perform different roles, and help direct the appropriate immune response for each different type of foreign object they encounter.

Although the general structure of all antibodies is very similar, a small region at the tip of the protein is extremely variable, allowing millions of antibodies with slightly different tip structures to exist. This region is known as the hypervariable region. Each of these variants can bind to a different target, known as an antigen. This huge diversity of antibodies allows the immune system to recognize an equally wide diversity of antigens. The part of the antigen recognized by an antibody is termed an “epitope.” These epitopes bind with their antibody in a highly specific interaction, called induced fit, which allows antibodies to identify and bind only their specific epitope in the matching antigen(s) in the midst of the millions of different molecules that make up an organism. Recognition of an antigen by an antibody tags it for attack by other parts of the immune system. Antibodies can also neutralize targets directly by, for example, binding to a part of a pathogen that it needs to cause an infection. Production of antibodies is the main function of the humoral immune system.

As used herein, the term “isolated” and the like means that the referenced material is free of components found in the natural environment in which the material is normally found. In particular, isolated biological material is free of cellular components. An isolated peptide or protein may be associated with other proteins or nucleic acids, or both, with which it associates in the cell, or with cellular membranes if it is a membrane-associated protein. An isolated material may be, but need not be, purified.

The term “substantially purified,” as used herein, refers to a molecule, such as a peptide, that is substantially free of cellular material (proteins, lipids, carbohydrates, nucleic acids), culture medium, chemical precursors, chemicals used in synthesis of the peptide, or combinations thereof. A peptide that is substantially purified has less than about 40%, 30%, 25%, 20%, 15%, 10%, 5%, 2%, 1% or less of the cellular material, culture medium, other polypeptides, chemical precursors, and/or chemicals used in synthesis of the peptide. Accordingly, a substantially pure molecule, such as a peptide, can be at least about 60%, 70%, 75%, 80%, 85%, 90%, 95%, 98%, or 99%, by dry weight, the molecule of interest.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation.

For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The current disclosure enhances the differential diagnosis and management of SARS-CoV-2 by establishing a new serologic assay platform for profiling a subject's pathogen exposure history, as well as providing peptides which are reactive with and sensitive to antibodies to SARS-CoV-2.

In contrast to molecular diagnostics where advances in technology such as polymerase chain reaction and high-throughput screening have dramatically improved sensitivity, specificity and breadth over the past 20 years, serologic methods remained largely unchanged. This lag is important given the role of serology in establishing the distribution and frequency of infection, testing the significance of association between the finding of an agent and disease, and in focusing efforts in pathogen discovery. Described herein is a sensitive, unbiased, highly multiplexed platform for diagnostic serology of SARS-CoV-2 in particular. This peptide array-based platform will enable new strategies for investigating the epidemiology and pathogenesis of acute and chronic diseases due to infection and for monitoring humoral responses to vaccines and immunomodulatory drugs. It will also serve as a screening tool for rapid selection of key informative peptides that can be used in established, inexpensive, alternative platforms including lateral flow immunoassays. Such applications will have practical utility for both clinical medicine and public health by enabling retrospective differential diagnosis of an infectious illness (when genetic footprints of the agent may no longer be present), and in facilitating outbreak investigation and surveillance.

This is particularly useful in infection by SARS-CoV-2 as a patient could no longer have the evidence of acute infection, e.g., viral nucleic acids, but have had a SARS-CoV-2 infection in the past. These assays will allow identification of individuals who are immune to future infection, capable of deployment to the workforce and who may serve as donors of antibodies for control of COVID-19, as well as for testing the effectiveness of vaccines for SARS-CoV-2.

Additionally, serodiagnosis of SARS-CoV-2 infection can be impeded by immunological cross-reactivity among the human coronaviruses (HCoVs): SARS-CoV-2;

SARS-CoV-1; MERS-CoV; OC43; 229E; HKU1; and NL63. Disclosed herein are SARS-CoV-2 peptides that enable discrimination between exposure to SARS-CoV-2 and other HCoVs.

A high-density peptide microarray and plasma samples collected at two time points from 50 subjects with SARS-CoV-2 infection confirmed by qPCR, samples collected in 2004-2005 from 11 subjects with IgG antibodies to SARS-CoV-1, 11 subjects with IgG antibodies to other seasonal human coronaviruses (HCoV), and 10 healthy human subjects were used. Through statistical modeling with linear regression and multidimensional scaling specific peptides were identified that were reassembled to identify 29 linear IgG SARS-CoV-2 epitopes that were immunoreactive with plasma from individuals who had asymptomatic, mild or severe SARS-CoV-2 infections, as well as 16 IgM SARS-CoV-2 epitopes and an additional 163 IgG epitopes for SARS-CoV-2. Also disclosed herein are highly specific epitopes for a human coronavirus other than SARS-CoV-2.

Thus disclosed herein is a sensitive, highly multiplexed, microarray-based assay that enabled the discrimination of antibody responses to linear epitopes specific to seven human coronaviruses: SARS-CoV-2; SARS; MERS; NL-63; OC-43; 229E; and HKU1 and the identification of 29 highly specific IgG epitopes, 16 IgM epitopes for SARS-CoV-2, and additional 163 IgG epitopes for SARS-CoV-2 and 53 highly specific epitopes for a human coronavirus other than SARS-CoV-2.

The work reported herein has been published in recently (see Mishra et, Immunoreactive peptide maps of SARS-CoV-2, Commun Biol 4, 225 (2021) (herein incorporated by reference in its entirety)).

### Isolated Peptides Specific and Sensitive for SARS-COV-2 Antibodies (SARS-COV-2 Reactive Peptides) and Other Human Coronavirus Antibodies (Human Coronavirus Reactive Peptides)

The present disclosure includes isolated peptides which are strongly reactive with, and sensitive to antibodies to SARS-CoV-2 in a patient sample. These peptides can be used in any type of serological assay or platform, now known or later developed, to screen for the presence of antibodies to SARS-CoV-2 and to determine if a subject has had an infection by and/or exposure to SARS-COV-2. These peptides can also be used to test for and monitor humoral responses to vaccines and immunomodulatory drugs, thus, being useful for the development of treatment and preventative agents for SARS-CoV-2.

One embodiment is an isolated peptide highly specific for SARS-CoV-2 listed in Table 2 (SEQ ID NOs: 1-29). A further embodiment is an isolated peptide highly specific for SARS-CoV-2 listed in Table 3 (SEQ ID NOs: 30-46). A further embodiment is an isolated peptide highly specific for SARS-CoV-2 listed in Table 4 (SEQ ID NOs: 46-208).

In further embodiments, isolated peptides highly specific for other human coronaviruses are also provided for herein. Isolated peptides highly specific for human coronavirus HKU1 are listed in Table 5 (SEQ ID NOs: 209-223). Isolated peptides highly specific for human coronavirus NL63 are listed in Table 5 (SEQ ID NOs: 224-233). Isolated peptides highly specific for human coronavirus OC43 are listed in Table 5 (SEQ ID NOs: 234-247). Isolated peptides highly specific for human coronavirus 229E are listed in Table 5 (SEQ ID NOs: 248-252). Isolated peptides highly specific for human coronavirus SARS-CoV-1 are listed in Table 5 (SEQ ID NOs: 253-261).

A further embodiment comprises a collection or set of peptides comprising amino acid sequences shifted one residue across any of the peptides listed in Table 2 (SEQ ID NOs: 1-26), Table 3 (SEQ ID NOs: 30-45), Table 4 (SEQ ID NOs: 46-208), and Table 5 (SEQ ID NOs: 209-261). This collection or set can contain peptides that are 6 amino acids in length, 7 amino acids in length, 8 amino acids in length, 9 amino acids in length, 10 amino acids in length, 11 amino acids in length, and up to 12 amino acids in length.

In one embodiment, each peptide in the set or collection is 12 amino acids in length. Other embodiments include a collection or set of isolated peptides comprising or consisting of amino acid sequences shifted one residue across one, two, three, four, five, six, seven, eight, nine, ten, eleven or all twelve of these twelve peptides. This collection or set can comprise or consist of isolated peptides comprising or consisting of 6 amino acids in length, 7 amino acids in length, 8 amino acids in length, 9 amino acids in length, 10 amino acids in length, 11 amino acids in length, and up to 12 amino acids in length.

Peptides of 12 amino acids were constructed based upon work that shows that antibodies bind to linear peptide sequences ranging from 5 to 9 amino acids in length and bind most efficiently when targets are flanked by additional amino acids (Buus et al. 2012). However, peptides containing less than 12 amino acids in length and more than 12 amino acids in length can be used. Peptides 13 amino acids in length, 14 amino acids in length, 15 amino acids in length, 16 amino acids in length, 17 amino acids in length, 18 amino acids in length, 19 amino acids in length, 20 amino acids in length, up to 25 amino acids in length, up to 30 amino acids in length, up to 35 amino acids in length, up to 40 amino acids in length, and up to 50 amino acids in length can be used.

In certain embodiments, the peptides described herein are produced by synthetic chemistry (i.e., a “synthetic peptide”). In other embodiments, peptides of the invention are produced biologically. An isolated peptide of the invention can be in water, a buffer, or in a dry form awaiting reconstitution, e.g., as part of a kit. An isolated peptide of the present invention can be in the form of a pharmaceutically acceptable salt. Suitable acids and bases that are capable of forming salts with the peptides of the present invention are well known to those of skill in the art and include inorganic and organic acids and bases.

In certain embodiments, the peptides described herein are modified. The peptides may be modified by a variety of techniques, such as by denaturation with heat and/or a detergent (e.g., SDS). Alternatively, peptides may be modified by association with one or more further moieties. The association can be covalent or non-covalent, and can be, for example, via a terminal amino acid linker, such as lysine or cysteine, a chemical coupling agent, or a peptide bond. The additional moiety can be, for example, a ligand, a ligand receptor, a fusion partner, a detectable label, an enzyme, or a substrate that immobilizes the peptide.

The disclosed peptides can be conjugated to a ligand, such as biotin (e.g., via a cysteine or lysine residue), a lipid molecule (e.g., via a cysteine residue), or a carrier protein (e.g., serum albumin, immunoglobulin Fc domain, keyhole limpet hemocyanin (KLH) via e.g., a cysteine or lysine residue). Attachment to ligands, such as biotin, can be useful for associating the peptide with ligand receptors, such as avidin, streptavidin, polymeric streptavidin, or neutravidin. Avidin, streptavidin, polymeric streptavidin, or neutravidin, in turn, can be linked to a signaling moiety (e.g., an enzyme, such as horse radish peroxidase (HRP) or alkaline phosphatase (ALP), or other moiety that can be visualized, such as a metallic nanoparticle or nanoshell (e.g., colloidal gold) or a fluorescent moiety), or a solid substrate (e.g., nitrocellulose membrane). Alternatively, the peptides of the invention can be fused or linked to a ligand receptor, such as avidin, streptavidin, polymeric streptavidin, or neutravidin, thereby facilitating the association of the peptides with the corresponding ligand, such as biotin and any moiety (e.g., signaling moiety) or solid substrate attached thereto. Examples of other ligand-receptor pairs are well-known in the art and can similarly be used.

The peptides can be fused to a fusion partner (e.g., a peptide or other moiety) that can be used to improve purification, to enhance expression of the peptide in a host cell, to aid in detection, and to stabilize the peptide. Examples of suitable compounds for fusion partners include carrier proteins (e.g., serum albumin, immunoglobulin Fc domain, KLH), and enzymes (e.g., horse radish peroxidase (HRP), beta-galactosidase, glutathione-S-transferase, alkaline phosphatase). The fusion can be achieved by means of a peptide bond. For example, peptides of the invention and fusion partners can be fusion proteins and can be directly fused in-frame or can comprise a peptide linker.

In addition, the disclosed peptides may be modified to include any of a variety of known chemical groups or molecules. Such modifications include, but are not limited to, glycosylation, acetylation, acylation, ADP-ribosylation, amidation, covalent attachment to polyethylene glycol (e.g., PEGylation), covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, ubiquitination, modifications with fatty acids, and transfer-RNA mediated addition of amino acids to proteins such as arginylation. Analogues of an amino acid (including unnatural amino acids) and peptides with substituted linkages are also included.

The disclosed peptides that consist of any of the sequences discussed herein may be modified by any of the discussed modifications. Such peptides still “comprise” or “consist of” the amino acids.

Modifications as set forth above are well-known to those of skill in the art and have been described in great detail in the scientific literature.

### Nucleic Acids Comprising a Sequence Encoding the SARS-CoV-2 Reactive Peptides and Other Human Coronavirus Reactive Peptides

In another aspect, the disclosure provides nucleic acids comprising a sequence encoding any disclosed peptide. Nucleic acids of the invention can be single- or double-stranded. A nucleic acid can be RNA, DNA, cDNA, genomic DNA, chemically synthesized RNA or DNA or combinations thereof. The nucleic acids can be purified free of other components, such as proteins, lipids and other polynucleotides. For example, the nucleic acids can be 50%, 75%, 90%, 95%, 96%, 97%, 98%, 99%, or 100% purified. The nucleic acids encode the peptides described herein. In certain embodiments, the nucleic acids encode a peptide having the sequence listed in Table 2 (SEQ ID NOs: 1-26), Table 3 (SEQ ID NOs: 30-45), Table 4 (SEQ ID NOs: 46-208) and/or Table 5 (SEQ ID NOs: 209-261). Nucleic acids can comprise other nucleotide sequences, such as sequences coding for linkers, signal sequences, TMR stop transfer sequences, transmembrane domains, or ligands useful in protein purification such as glutathione-S-transferase, histidine tag, and staphylococcal protein A.

Methods for preparing polynucleotides operably linked to an expression control sequence and expressing them in a host cell are well-known in the art. See, e.g., U.S. Pat. No. 4,366,246. A nucleic acid of the invention is operably linked when it is positioned adjacent to or close to one or more expression control elements, which direct transcription and/or translation of the polynucleotide.

Thus, for example, the peptides described herein can be produced recombinantly following conventional genetic engineering techniques. To produce a recombinant peptide, a nucleic acid encoding the peptide is inserted into a suitable expression system. Generally, a recombinant molecule or vector is constructed in which the polynucleotide sequence encoding the selected peptide is operably linked to an expression control sequence permitting expression of the peptide. Numerous types of appropriate expression vectors are known in the art, including, e.g., vectors containing bacterial, viral, yeast, fungal, insect or mammalian expression systems. Methods for obtaining and using such expression vectors are well-known. For guidance in this and other molecular biology techniques used for compositions or methods of the invention, see, e.g., Sambrook et al., Molecular Cloning, A Laboratory Manual, current edition, Cold Spring Harbor Laboratory, New York; Miller et al, Genetic Engineering, 8:277-298 (Plenum Press, current edition), Wu et al., Methods in Gene Biotechnology (CRC Press, New York, N.Y., current edition), Recombinant Gene Expression Protocols, in Methods in Molecular Biology, Vol. 62, (Tuan, ed., Humana Press, Totowa, N.J., current edition), and Current Protocols in Molecular Biology, (Ausabel et al., Eds.,) John Wiley & Sons, NY (current edition), and references cited therein.

Accordingly, the disclosure also provides vectors comprising nucleic acids described herein, and host cells comprising such vectors. In certain embodiments, the vector is a shuttle vector. In other embodiments, the vector is an expression vector (e.g., a bacterial or eukaryotic expression vector). In certain embodiments, the host cell is a bacterial cell. In other embodiments, the host cell is a eukaryotic cell.

### Platforms, Assays and Device for Using the SARS-CoV-2 Reactive Peptides and Other Human Coronavirus Reactive Peptides

There are a number of different conventional assays for detecting formation of an antibody-peptide complex comprising a peptide or peptides of the invention. For example, the detecting step can comprise performing a microarray assay, an ELISA assay, performing an immunofluorescence assay, performing a lateral flow immunoassay, performing an agglutination assay, performing a wavelength shift assay, performing a Western blot, slot blot, or dot blot, analyzing the sample in an analytical or centrifugal rotor, or analyzing the sample with an electrochemical, optical, or opto-electronic sensor. These different assays are described herein and/or are well-known to those skilled in the art.

Thus, the peptides disclosed herein can be used in any assay, format or platform for antibody detection including but not limited to microarrays, ELISA, RIA, lateral flow, western blot, and bead-based assays, as well as those platforms that are later developed.

In certain embodiments, the assay comprises immobilizing the antibody(s) in the sample; adding a peptide disclosed herein; and detecting the degree of antibody bound to the peptide, e.g., by the peptide being labeled or by adding a labeled substance, such as a labeled binding partner (e.g., streptavidin-HRP or streptavidin-colloidal gold complex) or a labeled antibody which specifically recognizes the peptide.

In other embodiments, the assay comprises immobilizing a peptide disclosed herein; adding the sample containing antibodies; and detecting the amount of antibody bound to the peptide, e.g., by adding another peptide disclosed herein conjugated, directly or indirectly, to a label (e.g., metallic nanoparticle or metallic nanoshell, fluorescent label, or enzyme (e.g., horseradish peroxidase or alkaline phosphatase)) or by adding a labeled substance, such as a binding partner or a labeled antibody which specifically recognizes the sample antibodies (e.g., anti-human IgG antibodies, or anti-human IgM antibodies).

In other embodiments, the assay comprises immobilizing a peptide disclosed herein; adding the sample containing antibodies; and detecting the amount of antibody bound to the peptide, e.g., by adding a first binding partner which specifically recognizes the sample antibodies (e.g., anti-human IgG antibodies, or anti-human IgM antibodies), and further adding a second binding partner, wherein the second binding partner is labeled and recognizes said first binding partner.

In still other embodiments, the assay comprises: reacting the peptide and the sample containing antibodies without any of the reactants being immobilized, and then detecting the amount of complexes of antibody and peptide, e.g., by the peptide being labeled or by adding a labeled substance, such as a labeled binding partner (e.g., streptavidin-HRP or streptavidin-colloidal gold complex) or a labeled antibody which specifically recognizes the peptide.

Immobilization of a peptide can be either covalent or non-covalent, and the non-covalent immobilization can be non-specific (e.g., non-specific binding to a polystyrene surface in a microtiter well). Specific or semi-specific binding to a solid or semi-solid carrier, support or surface, can be achieved by the peptide having, associated with it, a moiety which enables its covalent or non-covalent binding to the solid or semi-solid carrier, support or surface. For example, the moiety can have affinity to a component attached to the carrier, support or surface. In this case, the moiety may be, for example, a biotin or biotinyl group or an analogue thereof bound to an amino acid group of the peptide, and the component is then avidin, streptavidin, neutravidin, or an analogue thereof.

Suitable carriers, supports, and surfaces include, but are not limited to, metallic nanolayers, beads (e.g., magnetic beads, colloidal particles or metallic nanoparticles or nanoshells, such as colloidal gold, or particles or nanoparticles comprising silica, latex, polystyrene, polycarbonate, or PDVF), latex of co-polymers such as styrene-divinyl benzene, hydroxylated styrene-divinyl benzene, polystyrene, carboxylated polystyrene, beads of carbon black, non-activated or polystyrene or polyvinyl chloride activated glass, epoxy-activated porous magnetic glass, gelatin or polysaccharide particles or other protein particles, red blood cells, mono- or polyclonal antibodies or Fab fragments of such antibodies.

The protocols for immunoassays using antigens for detection of specific antibodies are well known in art. For example, a conventional sandwich assay can be used, or a conventional competitive assay format can be used.

Devices for performing specific binding assays, especially immunoassays, are known and can be readily adapted for use in the present methods. Solid-phase assay devices include microtiter plates, flow-through assay devices (e.g., lateral flow immunoassay devices), dipsticks, and immunocapillary or immunochromatographic immunoassay devices.

In embodiments, the solid or semi-solid surface or carrier is the floor or wall in a microtiter well, a filter surface or membrane (e.g., a nitrocellulose membrane or a PVDF (polyvinylidene fluoride) membrane), a hollow fiber, a beaded chromatographic medium (e.g., an agarose or polyacrylamide gel), a magnetic bead, a fibrous cellulose matrix, an HPLC matrix, an FPLC matrix, a substance having molecules of such a size that the molecules with the peptide bound thereto, when dissolved or dispersed in a liquid phase, can be retained by means of a filter, a substance capable of forming micelles or participating in the formation of micelles allowing a liquid phase to be changed or exchanged without entraining the micelles, a water-soluble polymer, or any other suitable carrier, support or surface.

In some embodiments, the peptide is provided with a suitable label which enables detection. Conventional labels may be used which are capable, alone or in concert with other compositions or compounds, of providing a detectable signal. Suitable labels include, but are not limited to, enzymes (e.g., HRP, beta-galactosidase, or alkaline phosphatase), fluorescent labels, radioactive labels, colored latex particles, and metal-conjugated labels (e.g., metallic nanolayers, metallic nanoparticle- or metallic nanoshell-conjugated labels). Suitable metallic nanoparticle or metallic nanoshell labels include, but are not limited to, gold particles, silver particles, copper particles, platinum particles, cadmium particles, composite particles, gold hollow spheres, gold-coated silica nanoshells, and silica-coated gold shells. Metallic nanolayers suitable for detectable layers include nanolayers comprised of cadmium, zinc, mercury, and noble metals, such as gold, silver, copper, and platinum.

Suitable detection methods include, but are not limited to, detection of an agent which is tagged, directly or indirectly, with a colorimetric assay (e.g., for detection of HRP or beta-galactosidase activity), visual inspection using light microscopy, immunofluorescence microscopy, including confocal microscopy, or by flow cytometry (FACS), autoradiography (e.g., for detection of a radioactively labeled agent), electron microscopy, immunostaining, subcellular fractionation, or the like. In one embodiment, a radioactive element (e.g., a radioactive amino acid) is incorporated directly into a peptide chain. In another embodiment, a fluorescent label is associated with a peptide via biotin/avidin interaction, association with a fluorescein conjugated antibody, or the like. In one embodiment, a detectable specific binding partner for the antibody is added to the mixture. For example, the binding partner can be a detectable secondary antibody or other binding agent (e.g., protein A, protein G, protein L or combinations thereof) which binds to the first antibody. This secondary antibody or other binding agent can be labeled with, for example, a radioactive, enzymatic, fluorescent, luminescent, metallic nanoparticle or metallic nanoshell (e.g. colloidal gold), or other detectable label, such as an avidin/biotin system. In another embodiment, the binding partner is a peptide disclosed herein, which can be conjugated directly or indirectly to an enzyme, such as horseradish peroxidase or alkaline phosphatase or other signaling moiety. In such embodiments, the detectable signal is produced by adding a substrate of the enzyme that produces a detectable signal, such as a chromogenic, fluorogenic, or chemiluminescent substrate.

In some embodiments, the detection procedure comprises visibly inspecting the antibody-peptide complex for a color change or inspecting the antibody-peptide complex for a physical-chemical change. Physical-chemical changes may occur with oxidation reactions or other chemical reactions. They may be detected by eye, using a spectrophotometer, or the like.

One assay format is a lateral flow immunoassay format. Antibodies to human or animal immunoglobulins, can be labeled with a signal generator or reporter (e.g., colloidal gold) that is dried and placed on a glass fiber pad (sample application pad or conjugate pad). The diagnostic peptide is immobilized on membrane, such as nitrocellulose or a PVDF (polyvinylidene fluoride) membrane. When a sample is applied to the sample application pad (or flows through the conjugate pad), it dissolves the labeled reporter, which then binds to all antibodies in the sample. The resulting complexes are then transported into the next membrane (PVDF or nitrocellulose containing the diagnostic peptide) by capillary action. If antibodies against the diagnostic peptide are present, they bind to the diagnostic peptide striped on the membrane, thereby generating a signal (e.g., a band that can be seen or visualized). An additional antibody specific to the labeled antibody or a second labeled antibody can be used to produce a control signal.

An alternative format for the lateral flow immunoassay comprises the peptides or compositions being conjugated to a ligand (e.g., biotin) and complexed with labeled ligand receptor (e.g., streptavidin-colloidal gold). The labeled peptide complexes can be placed on the sample application pad or conjugate pad. Anti-human IgG/IgM or anti-animal IgG/IgM antibodies are immobilized on a membrane, such as nitrocellulose of PVDF, at a test site. When sample is added to the sample application pad, antibodies in the sample react with the labeled peptide complexes such that antibodies that bind to peptides become indirectly labeled. The antibodies in the sample are then transported into the next membrane (PVDF or nitrocellulose containing the diagnostic peptide) by capillary action and bind to the immobilized anti-human IgG/IgM or anti-animal IgG/IgM antibodies. If any of the sample antibodies are bound to the labeled peptides, the label associated with the peptides can be seen or visualized at the test site.

Another assay for the screening of blood products or other physiological or biological fluids is an enzyme linked immunosorbent assay, i.e., an ELISA. Typically in an ELISA, isolated peptides or collection or set of peptides disclosed herein, are adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of nonfat dry milk which also contains a preservative, salts, and an antifoaming agent). The well is then incubated with a biological sample suspected of containing specific antibody. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody that is conjugated to an enzyme or other label by standard procedures and is dissolved in blocking buffer. The label can be chosen from a variety of enzymes, including horseradish peroxidase (HRP), beta-galactosidase, alkaline phosphatase (ALP), and glucose oxidase. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length). Conditions for performing ELISA assays are well-known in the art.

In another embodiment of an ELISA, a peptide or a collection or set of peptides disclosed herein is immobilized on a surface, such as a ninety-six-well ELISA plate A sample is then added and the assay proceeds as above.

In still other embodiments, a peptide or collection or set of peptides disclosed herein are electro- or dot-blotted onto nitrocellulose paper. Subsequently, a sample, such as a biological fluid (e.g., serum or plasma) is incubated with the blotted antigen, and antibody in the biological fluid is allowed to bind to the antigen(s). The bound antibody can then be detected, e.g., by standard immunoenzymatic methods or by visualization using metallic nanoparticles or nanoshells coupled to secondary antibodies or other antibody binding agents or combinations thereof.

It should be understood by one of skill in the art that any number of conventional protein assay formats, particularly immunoassay formats, may be designed to utilize the isolated peptides, and collections and sets of peptides disclosed herein, for the detection of SARS-CoV-2 and/or other human coronavirus antibodies in a subject. Thus, the use of the peptides is thus not limited by the selection of the particular assay format, and is believed to encompass assay formats that are known to those of skill in the art. To date most serology has been performed using singleplex ELISA, complement fixation or neutralization assays. More recently, Luminex-based systems have been employed that can address up to 100 antigenic targets simultaneously (i.e., 100 individual pathogens, 100 individual antigenic targets for one pathogen, or some variation thereof). Additionally, arrays are established that comprise spotted recombinant proteins expressed in vitro in E. coli, S. cerevesiae, baculoviruses, or cell-free, coupled transcription-translation.

One goal of the present disclosure is to automate the process of SARS-CoV-2 antibody detection and make it inexpensive, quick and accurate as well as detect exposure per se rather than to rigorously characterize humoral responses to specific pathogens.

One assay that meets these requirements is a programmable peptide array.

The peptide array capacity can be exploited to print multiple arrays per glass slide (configurations of 1, 3, 8, or 12 arrays can be printed).

Thus, one embodiment is a peptide microarray comprising peptides that are reactive with, and specific for SARS-CoV-2 antibodies. In some embodiments the peptide microarray comprises: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof.

A further embodiment is a peptide microarray comprising peptide or peptides which are reactive with, and specific for SARS-CoV-2 antibodies and/or other human coronaviruses comprising: a peptide or peptides chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptides chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof.

In some embodiments the peptide array comprises a set or collection of peptides, comprising amino acid sequences shifted one residue across all of the peptides comprising or consisting of the amino acid sequences of SEQ ID NOs: 1-29 and/or SEQ ID NOs: 30-45 and/or SEQ ID NOs: 46-208 and/or SEQ ID NOs: 209-261.

### Methods and Systems for Serological Detection of Exposure to SARS-CoV-2

The present disclosure also includes methods and systems for the detection of exposure to antigens of SARS-CoV-2, i.e., antibodies to SARS-CoV-2, in any sample utilizing the various peptides, isolated and non-isolated, and peptide microarrays disclosed herein.

Suitable methods typically include: receiving or obtaining (e.g., from a patient) a sample of bodily fluid or tissue likely to contain antibodies; contacting (e.g., incubating or reacting) a sample to be assayed with a peptide or peptides of the invention, under conditions effective for the formation of a specific peptide-antibody complex (e.g., for specific binding of the peptide to the antibody); and assaying the contacted (reacted) sample for the presence of an antibody-peptide reaction (e.g., determining the amount of an antibody-peptide complex). The presence of an elevated amount of the antibody-peptide complex indicates that the subject was exposed to and infected by SARS-CoV-2. A peptide, including a modified form thereof, which “binds specifically” to an antibody against a SARS-CoV-2 antigen interacts with the antibody, or forms or undergoes a physical association with it, in an amount and for a sufficient time to allow detection of the antibody.

Conditions for reacting peptides and antibodies so that they react specifically are well-known to those of skill in the art. See, e.g., Current Protocols in Immunology (Coligan et al., editors, John Wiley & Sons, Inc.).

Once the disclosed peptide or peptides and sample antibody are permitted to react in a suitable medium, an assay is performed to determine the presence or absence of an antibody-peptide reaction. Any of the assays discussed herein can be used.

The methods and systems of the present disclosure may be used to detect exposure to antigens of SARS-CoV-2 in research and clinical settings.

One sample for use in the methods is a biological sample. A biological sample may be obtained from a tissue of a subject or bodily fluid from a subject including but not limited to nasopharyngeal aspirate, blood, cerebrospinal fluid, saliva, serum, plasma, urine, sputum, bronchial lavage, pericardial fluid, or peritoneal fluid, or a solid such as feces. The subject may be any animal, particularly a vertebrate and more particularly a mammal, including, without limitation, a cow, dog, human, monkey, mouse, pig, or rat. In one embodiment, the subject is a human.

A sample may also be a research, clinical, or environmental sample, such as cells, cell culture, cell culture medium, and compositions for use as, or the development of pharmaceutical and therapeutic agents.

Additional applications include, without limitation, detection of the screening of blood products (e.g., screening blood products for infectious agents), biodefense, food safety, environmental contamination, forensics, and genetic-comparability studies. The present disclosure also provides methods and systems for detecting viral antibodies in cells, cell culture, cell culture medium and other compositions used for the development of pharmaceutical and therapeutic and immunomodulatory agents.

The subject may have been exposed to antigens of SARS-CoV-2, suspected of having exposure to antigens of SARS-CoV-2 or believed not to have had exposure to antigens of SARS-CoV-2.

In one embodiment, the subject is being tested for use of their plasma for treatment of COVID-19 patients.

In one embodiment, the subject may be a test subject, which has been administered a SARS-CoV-2 vaccine or immunomodulatory agent.

The systems and methods described herein support the detection and measure of a humoral immune response to SARS-CoV-2.

One embodiment provides a system for the detection of exposure to antigens of SARS-CoV-2, i.e., antibodies to SARS-CoV-2, in any sample. The system includes at least one subsystem, wherein the subsystem includes a peptide or peptides or a collection or set of peptides disclosed herein, which are reactive with, and specific for SARS-CoV-2 antibodies, comprising: a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof. The system can also include additional subsystems for the purpose of: preparation of the sample; binding of any SARS-CoV-2 antibody in the sample with the peptides(s); washing the unbound sample; and visualization and/or quantification of bound antibody or antibodies.

A further embodiment provides a system for the detection of exposure to antigens of SARS-CoV-2, i.e., antibodies to SARS-CoV-2, in any sample. The system includes at least one subsystem, wherein the subsystem includes a peptide microarray comprising a peptide or peptides or a collection or set of peptides disclosed herein, which are reactive with, and specific for SARS-CoV-2 antibodies comprising: a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof. The system can also include additional subsystems for the purpose of: preparation of the sample; binding of any SARS-CoV-2 antibody in the sample with the peptides(s); washing the unbound sample; and visualization and/or quantification of bound antibody or antibodies.

Another embodiment provides a system for the differential detection of exposure to antigens of SARS-CoV-2, i.e., antibodies to SARS-CoV-2 and other human coronaviruses, in any sample. The system includes at least one subsystem, wherein the subsystem includes a peptide or peptides or a collection or set of peptides disclosed herein, which are reactive with, and specific for SARS-CoV-2 and other human coronavirus antibodies, comprising: a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptide chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof. The system can also include additional subsystems for the purpose of: preparation of the sample; binding of any antibody in the sample with the peptides(s); washing the unbound sample; and visualization and/or quantification of bound antibody or antibodies.

A further embodiment provides a system for the differential detection of exposure to antigens of SARS-CoV-2, i.e., antibodies to SARS-CoV-2 and other human coronaviruses, in any sample. The system includes at least one subsystem, wherein the subsystem includes a peptide microarray comprising a peptide or peptides or a collection or set of peptides disclosed herein, which are reactive with, and specific for SARS-CoV-2 and other human coronavirus antibodies comprising: peptide or peptides or a collection or set of peptides disclosed herein, which are reactive with, and specific for SARS-CoV-2 and other human coronavirus antibodies, comprising: a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptide chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof. The system can also include additional subsystems for the purpose of: preparation of the sample; binding of any antibody in the sample with the peptides(s); washing the unbound sample; and visualization and/or quantification of bound antibody or antibodies.

The present disclosure provides a method for detecting the exposure to antigens of SARS-CoV-2, i.e., antibodies to SARS-CoV-2, in any sample, including the steps of: contacting the sample with a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof, under conditions sufficient for any antibodies to SARS-CoV-2 in the sample and the peptides to bind; and visualizing and/or quantifying any bound antibody or antibodies to the peptides. The method may optionally include a step for washing any unbound sample.

The present disclosure provides a method for differentially detecting the exposure to antigens of SARS-CoV-2 and other human coronaviruses, i.e., antibodies to SARS-CoV-2 and other human coronaviruses, in any sample, including the steps of: contacting the sample with a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptide chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof, under conditions sufficient for any antibodies to SARS-CoV-2 and/or other human coronaviruses in the sample and the peptides to bind; and visualizing and/or quantifying any bound antibody or antibodies to the peptides. The method may optionally include a step for washing any unbound sample.

The present disclosure further provides a method for detecting the exposure to antigens of SARS-CoV-2, i.e., antibodies to SARS-CoV-2, in any sample, including the steps of: contacting the sample with a peptide microarray comprising a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); or combinations thereof, under conditions sufficient for any antibodies to SARS-CoV-2 in the sample and the peptides to bind; and visualizing and/or quantifying any bound antibody or antibodies to the peptides. The method may optionally include a step for washing any unbound sample.

The present disclosure further provides a method for differentially detecting the exposure to antigens of SARS-CoV-2 and other human coronaviruses, i.e., antibodies to SARS-CoV-2 and other human coronaviruses, in any sample, including the steps of: contacting the sample with a peptide microarray comprising a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptide chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof, under conditions sufficient for any antibodies to SARS-CoV-2 and/or other human coronaviruses in the sample and the peptides to bind; and visualizing and/or quantifying any bound antibody or antibodies to the peptides. The method may optionally include a step for washing any unbound sample.

Any method of detection discussed herein or known in the art can be used for visualizing and/or quantifying the bound antibodies.

### Kits

The present disclosure also includes reagents and kits for practicing the methods of the invention. These reagents and kits may vary.

One reagent of the kit would be a peptide or peptides or a collection or set of peptides disclosed herein, which are reactive with, and specific for SARS-CoV-2 and/or other human coronavirus antibodies, comprising: a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptide chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof.

In certain embodiments, the peptides are attached to or immobilized on a solid support. In some embodiments, the peptides are attached to or immobilized on a solid support through a metallic nanolayer (e.g., cadmium, zinc, mercury, gold, silver, copper, or platinum nanolayer). In certain embodiments, the solid support is a bead (e.g., a colloidal particle or a metallic nanoparticle or nanoshell), a flow path in a lateral flow immunoassay device, a flow path in an analytical or centrifugal rotor, a tube or a well (e.g., in a plate), or a sensor (e.g., an electrochemical, optical, or opto-electronic sensor).

Reagents for particular types of assays can also be provided in kits disclosed herein. Thus, the kits can include a population of beads (e.g., suitable for an agglutination assay or a lateral flow assay), or a plate (e.g., a plate suitable for an ELISA assay). In other embodiments, the kits comprise a device, such as a lateral flow immunoassay device, an analytical or centrifugal rotor, a Western blot, a dot blot, a slot blot, or an electrochemical, optical, or opto-electronic sensor.

In one particular embodiment, the kit would comprise peptide microarrays comprising a peptide or peptides or a collection or set of peptides, which are reactive with, and specific for SARS-CoV-2 and/or other human coronavirus antibodies, comprising: a peptide or peptide chosen from the group consisting of the peptides listed in Table 2 (SEQ ID NOs: 1-29); and/or a peptide or peptides chosen from the group consisting of the peptides listed in Table 3 (SEQ ID NOs: 30-45); and/or a peptide or peptides chosen from the group consisting of the peptides listed in Table 4 (SEQ ID NOs: 46-208); and/or a peptide or peptide chosen from the group consisting of the peptides listed in Table 5 (SEQ ID NOs: 209-261); and/or a collection or set of peptides shifted one residue across at least one peptide chosen from the group consisting of the peptides listed in Table 2 (SEQ ID NOs: 1-29); and/or a collection or set of peptides shifted one residue across at least one peptide chosen from the group consisting of the peptides listed in Table 3 (SEQ ID NOs: 30-45); and/or a collection or set of peptides shifted one residue across at least one peptide chosen from the group consisting of the peptides listed in Table 4 (SEQ ID NOs: 46-208); and/or a collection or set of peptides shifted one residue across at least one peptide chosen from the group consisting of the peptides listed in Table 5 (SEQ ID NOs: 209-261); or combinations thereof.

In addition, the kits can include various diluents and buffers, labeled conjugates or other agents for the detection of specifically bound antigens or antibodies (e.g. labeling reagents), and other signal-generating reagents, such as enzyme substrates, cofactors and chromogens. In some embodiments, the kit comprises an anti-human IgG/IgM antibody conjugated to a detectable label (e.g., a metallic nanoparticle, metallic nanoshell, metallic nanolayer, fluorophore, colored latex particle, or enzyme) as a labeling reagent. In other embodiments, the kit comprises protein A, protein G, protein A/G fusion proteins, protein L, or combinations thereof conjugated to a detectable label (e.g., a metallic nanoparticle, metallic nanoshell, metallic nanolayer, fluorophore, colored latex particle, or enzyme) as a labeling reagent. In still other embodiments, the labeling reagents of the kit are a second collection or set of peptides of the invention conjugated to a detectable label (e.g., a metallic nanoparticle, metallic nanoshell, metallic nanolayer, fluorophore, colored latex particle, or enzyme). The second collection or set of peptides can be the same as or different than the collection or set of peptides, which may optionally be attached to or immobilized upon a solid support.

Other components of a kit can easily be determined by one of skill in the art. Such components may include coating reagents, polyclonal or monoclonal capture antibodies specific for a peptide of the invention, or a cocktail of two or more of the antibodies, purified or semi-purified extracts of these antigens as standards, monoclonal antibody detector antibodies, an anti-mouse, anti-dog, anti-cat, anti-chicken, or anti-human antibody conjugated to a detectable label, indicator charts for colorimetric comparisons, disposable gloves, decontamination instructions, applicator sticks or containers, a sample preparatory cup and buffers or other reagents appropriate for constituting a reaction medium allowing the formation of a peptide-antibody complex.

Such kits provide a convenient, efficient way for a clinical laboratory to diagnose infection by corona virus. Thus, in certain embodiments, the kits further comprise instructions.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods

### Generation of Human Peptide Array-HCoV SeroChip

The platform for epitope discovery was a programmable peptide microarray that can accommodate up to 3 million distinct linear peptides on a 75 mm×26 mm slide (Nimblegen-Roche). The array can also be divided into 12 subarrays, each containing approximately 173,000 “12-mer peptides”. The 12-mer format was based on the observation that serum antibodies bind linear peptide sequences ranging from 5 to 9 amino acid (aa) and bind most efficiently when targets are flanked by additional amino acids (Buus et al. 2012). To enable differential detection of antibodies specific for SARS-CoV-2 infections, a custom NCBI GenBank and VIPR database for full proteomes of seven human coronaviruses: SARS-CoV-2; SARS; MERS; NL-63; OC-43; 229E; and HKU1, was created (Table 1). Two bat coronavirus proteomes similar to SARS-CoV-2 were also included (Zhou et al. 2020). 1000 randomly selected 12 amino acid long scrambled peptides were added for background correction and nonspecific binding of peptides.

For each virus selected, all available protein sequences available before January 2020 from the NCBI and Virus Pathogen Database and Analysis Resource (VIPR) protein databases were downloaded and then a peptide database created comprising overlapping 12-mer peptides that tiled the whole proteome of each of these agents with 11 amino acid (aa) overlap in a sliding window pattern (Tokarz et al. 2020; Tokarz et al. 2018; Mishra et al. 2019; Mishra et al. 2018). The selected peptide sequences were passed through a redundancy filter to yield 283,630 unique peptide sequences and 24,779,921 non-unique (present in more than one virus) for a total of 172,665 peptides. Redundant peptides were excluded prior to synthesis. The individual peptides in the library were printed in random positions on the peptide array to minimize the impact of locational bias.

TABLE 1
Design and Characteristics of HCoV-peptide Array

| | | | Total | Total |
|---|---|---|---|---|
| | | No. of | redundant | unique |
| | | protein | “12-aa” | “12-aa” |
| | Virus database | sequences | peptides | peptides |
| 1 | SARS-CoV-2-MN908947-CDS | 10 | 9591 | 9591 |
| 2 | Human-229E-CoV-PDB | 662 | 576999 | 15756 |
| 3 | Human-HKU1-CoV-PDB | 721 | 554447 | 16390 |
| 4 | Human-NC63-CoV-PDB | 1320 | 1000287 | 16392 |
| 5 | Human-OC43-CoV-PDB | 2667 | 2679272 | 29635 |
| 6 | MERS-CoV-PDB | 7253 | 8604059 | 54188 |
| 7 | SARS-CoV-PDB | 1996 | 1484140 | 29791 |
| 8 | Human-CoV-variants-VIPR-PDB | 12131 | 9880717 | 101145 |
| 9 | Bat-CoV-ZC45 | 11 | 9678 | 9678 |
| 10 | Bat-CoV-ZXC21 | 11 | 9655 | 9655 |
| 11 | Redundant-non-specific-peptides | NA | NA | 1000 |

| | | |
|---|---|---|
| Total number of unique and redundant peptides | 24779921 | 283630 |

| | |
|---|---|
| Total peptides in HCOV-SeroChip | 172665 |

### Samples and Experimental Design

The study was approved by the Medical Ethical Committee of Sun Yat-Sen University (approval number 2020-060). An informed and written consent was obtained by patients.

A total 132 plasma samples were tested and analyzed using HCoV peptide arrays. Samples were divided into six groups: group 1] COVID-19 patients with non-severe (mild) disease (n=22); group 2] COVID-19 patients with severe disease (n=22); group 3] patients with SARS-CoV-2 infections but no-symptoms (asymptomatic COVID-19) (n=6); group 4] SARS-2003 IgG positive cases (n=11); group 5] other banked HCoV IgG positive controls (n=11); and group 6] healthy controls (n=10). The average age was 44.0±16.73 years for the mild COVID-19 group, 60.1±12.37 years for the severe COVID-19 group, and 43.5±15.08 years for the asymptomatic COVID-19 group. The asymptomatic group subjects were family members or close contacts of the mild or severely ill patients. The average age for SARS-CoV-1 IgG positive control group was 24.4±5.0 years. The average age for healthy control group was 46.3±7.3 years. Other HCoV controls included banked samples for which age and sex information was not available.

Plasma samples were collected at two different time points, a minimum of two weeks apart, from group COVID-19 patients (groups 1, 2 and 3). The first time point (early) was collected at 12.9±5.9 post onset of disease (POD) for the mild disease group, and at 9.6±3.5 days POD for the severe disease group. The second time point (late) was collected at 34.7±8.3 days POD for the mild disease group, and at 24.8±6.8 days POD for the severe disease group. For asymptomatic group, the first time point was collected on the day of hospitalization and the second time point was collected at 14.5±4.6 days after the day of hospitalization. Non COVID-19 samples from control groups (group 4, group 5 and group 6), were from adults without any evidence or history of infection with SARS-CoV-2.

All COVID-19 patients were tested for SARS-CoV-2 RNA in respiratory specimens using the China FDA approved Novel Coronavirus (2019-nCoV) Real Time RT-PCR kit from LifeRiver Ltd. (Catalog #: RR-0479-02) real-time RT-PCR (CDC 2003). The diagnosis of COVID-19 pneumonia, and severity criteria were assessed at Guangdong CDC based on the New Coronavirus Pneumonia Prevention and Control Program (6th edition) published by the National Health Commission of China (Wu et al. 2020).

### HCOV Peptide Array Synthesis, Sample Binding and Processing

Peptide synthesis was accomplished by light-directed array synthesis in a Nimble therapeutics Maskless Array Synthesizer (MAS) using an amino-functionalized substrate coupled with 6-amino hexanoic acid as a spacer and amino acid derivatives carrying a photosensitive 2-(2-Nitrophenyl) propyl-oxy-carbonyl-group (NPPOC) protection group (Orgentis Chemicals) Amino acids (final concentration 20 mM) were pre-mixed for 10 minutes in N,N-Dimethylformamide (DMF, Sigma Aldrich) with N,N,N′,N′-Tetramethyl-O-(1H-benzotriazol-1-yl) uroniumhexafluorophosphate (HBTU, Protein Technologies, Inc.; final concentration 20 mM) as an activator, 6-Chloro-1-hydroxybenzotriazole (6-Cl-HOBt, Protein Technologies, Inc.; final concentration 20 mM) to suppress racemization, and N,N-Diisopropylethylamine (DIPEA, Sigma Aldrich; final concentration 31 mM) as a base. Activated amino acids were then coupled to the array surface for 3 minutes. Following each coupling step, the microarray was washed with N-methyl-2-pyrrolidone (VWR International), and site-specific cleavage of the NPPOC protection group was accomplished by irradiation of an image created by a Digital Micro-Mirror Device (Texas Instruments), projecting 365 nm wavelength light. Coupling cycles were repeated to synthesize the full in silico-generated peptide library.

Eleven arrays were used to test 132 plasma samples. Before loading, plasma samples were heat inactivated at 56° C. for 30 minutes. Plasma samples were diluted (1:50) with binding buffer (0.1M Tris-Cl, 1% alkali soluble casein, 0.05% Tween-20, and water). The peptide arrays were incubated overnight at 4° C. on a flat surface with individual sample/sub-array. Overnight sample incubation was followed by three 10-minute washes with 1×TBS-T (0.05% Tween-20) at room temperature (RT). Secondary antibodies IgG (cat no. 109-605-098, Alexa Fluor 647-AffiniPure Goat Anti-Human IgG, Fcy fragment specific, Jackson ImmunoResearch Labs) and IgM (cat no. 109-165-129, Cy™3 AffiniPure Goat Anti-Human IgM, Jackson ImmunoResearch Labs) were diluted in 1×PBS at a concentration of 0.1 μg/ml, and arrays were incubated in Plastic Coplin Jar (cat no. 590130, Fisher Scientific) for 3 hours at RT with gentle shaking. Secondary antibody incubation was also followed by three 10-minute washes with 1×TBST at RT. After a final wash, the arrays were dried and scanned on a microarray scanner at 2-μm resolution, with an excitation wavelength of 635 nm (IgG) and 532 nm (IgM). The images were analyzed using the PepArray analysis program. The fluorescent signals were converted into arbitrary unit (AU) intensity plots ranging minimum to maximum intensity 0-65,000 AU.

### Quality and Reproducibility of the Array Data

In the array synthesis process, Nimble Therapeutics uses a quality control step that builds thousands of peptides with known binding epitopes to streptavidin, which have been confirmed through SPR and crystallization method (Lyamichev et al. 2017). Each subarray on the entire synthesized array slide contained these quality control peptides in addition to the customized targeted experimental peptides. Additionally, every array slide was QC analyzed and the signal AU from these control peptides for each slide was correlated to the banked data from previous QC analyses. Arrays that did not meet standard cutoff thresholds were deemed as failed and removed from the further experimental process. Random nonadjacent tiling of overlapping peptides also enhanced confidence in the probe quality and supported the reactivity of an individual epitope by having multiple, overlapping peptides per epitope. In our previous studies, epitope reactivity of 12 aa overlapping peptides with an 11 amino acid overlap (single aa tiling) has been shown.

The reproducibility of data and inter-array reproducibility analysis of peptide arrays have been reported in previous studies by testing technical replicates on two separate microarrays. The two technical replicates for the target epitopes showed almost identical results with distinct epitopes (Tokarz et al. 2018; Mishra et al. 2018; Forsstrom et al. 2014).

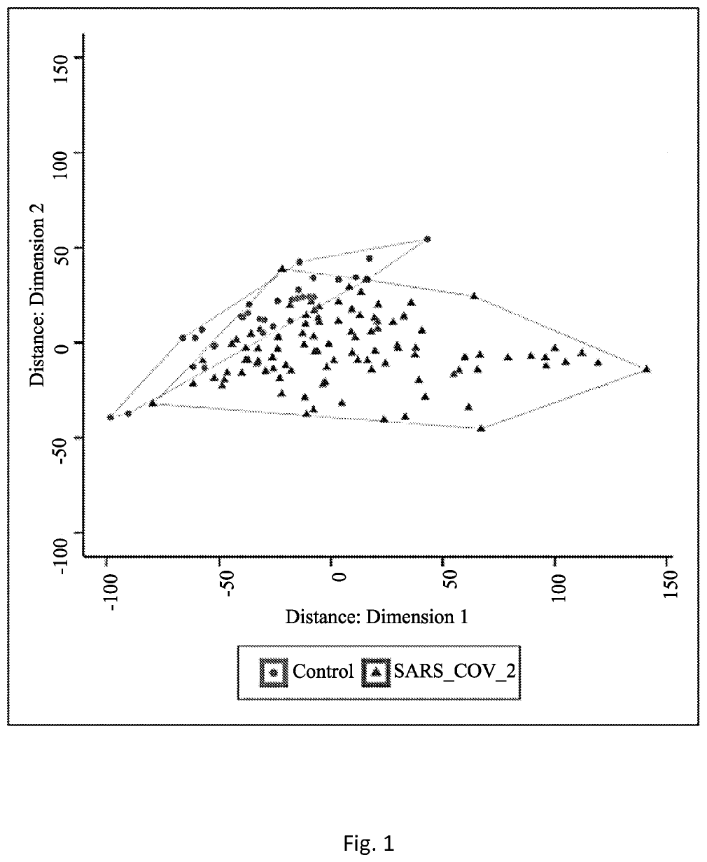

All analysis similar to IgG was also performed to generate data from IgM reactive peptides. For IgM analysis, >10,000 AU filtration step reduced the initial number of peptides from 172,665 to 24,728 for further analysis. A total of 10,816 peptides (7144 from the COVID-19 group and 3672 from the control group) peptides yielded group-specific differences (p<0.05) in signal intensity. MDS efforts did not separate samples collected from patients with COVID-19 and controls (FIG. 1). However, the presence of three continuous peptides that were reactive in samples from SARS-CoV-2-infected subjects (irrespective of disease status) but not in control groups, allowed the identification of 16 SARS-CoV-2-specific IgM epitopes (Table 3).

### Statistics and Reproducibility

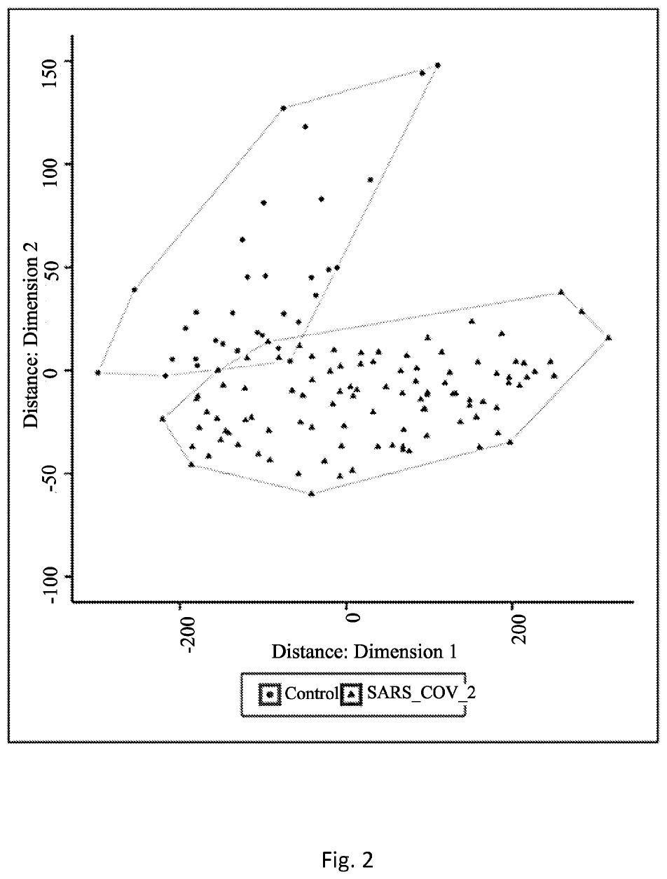

Reactivity values for all 132 samples were pooled together and peptides showing >10,000 AU for any samples were retained for further analysis. Regression analysis, fold-changes, and standard errors were estimated by fitting a linear model for signal intensities generated by each peptide, applying empirical Bayesian smoothing to the standard errors, and then determining those peptides that yielded statistically significant signal by contrasting linear models for each peptide between SARS-CoV-2 and Control samples at a significance value of <0.0520. edgeR package in R was used for this purpose. It implements quantile-adjusted conditional maximum-likelihood method for estimating dispersions followed by fitting negative binomial generalized linear models. This was followed by a quasi-likelihood F-test to determine those peptides that yielded statistically significant signal by contrasting linear models for each peptide between SARS-CoV-2 and Control samples. MDS plots (FIG. 2) shows the discriminative ability of these peptides. These peptides were then used to reassemble longer epitope sequences (Table 2, Table 3). The normalization, background correction was performed using preprocessCore R package (Bolstad et al. 2003) and statistical comparison of peptide microarray intensities between groups were performed using the edgeR package (Robinson et al. 2010).

The analysis was performed to differentiate peptides that were immunoreactive with COVID-19 patients (groups 1, 2, and 3) and versus control groups (groups 4, 5, and 6) samples. MDS plots were generated using signal data for these significant peptides. The code for reassembly and plots was prepared using Rstudio v 1.2.5019. The plots were generated using ggplot2 package. A custom color-blind friendly color pallete was used to make the plots. Alignment of reactive epitopes on SARS-CoV-2 proteome was performed using Geneious version 10.0.9.

### Example 2—Results

As described in Example 1, plasma samples were heat-inactivated at 56° C. for 30 minutes, diluted (1:50), added to HCoV peptide arrays, incubated with Alexa Fluor 647 labeled goat anti-human IgG, Cy™3 labeled goat anti-human IgM antibodies, and scanned on a microarray scanner.

Fluorescence signal data for all the peptides from IgG and IgM scanned images of all HCoV peptide arrays was converted to arbitrary units (AU), pooled, background corrected, and normalized to avoid any inter-experimental variations (Tokarz et al. 2018; Mishra et al. 2018; Buus et al. 2012). A peptide signal was considered reactive if the intensity reading (AU) was above the threshold (mean±2 SD readings of random peptides, >10,000 AU for IgG and IgM analysis). A cutoff threshold for peptide recognition was defined as mean ±2 times the standard deviation of the mean intensity value of all negative controls (Valentini et al. 2017). The regression analysis was performed to measure fold-changes on normalized and background corrected data for filtered peptides and followed by multidimensional scaling (MDS) analysis.

For IgG analysis, the >10,000 AU filtration step reduced the initial number of peptides from 172,665 to 79,714 for further analysis. A total of 37,237 peptides (18,533 from the COVID-19 group and 18,704 from the control group) were identified by regression analysis. This yielded group specific differences (p<0.05) in signal intensity. MDS analysis was performed for IgG and IgM antibodies to differentiate peptides that were immunoreactive with COVID-19 patients (Groups 1-3) versus control groups (Group 4-6). MDS analysis of these peptides confirmed separation of patients with COVID-19 (Groups 1-3) and controls (Groups 4-6) into separate clusters with minimal overlap (FIG. 2). Out of 18,533 reactive 12-mer peptides associated with the COVID-19 group, 981 peptides were specific for the SARS-CoV-2 polyprotein.

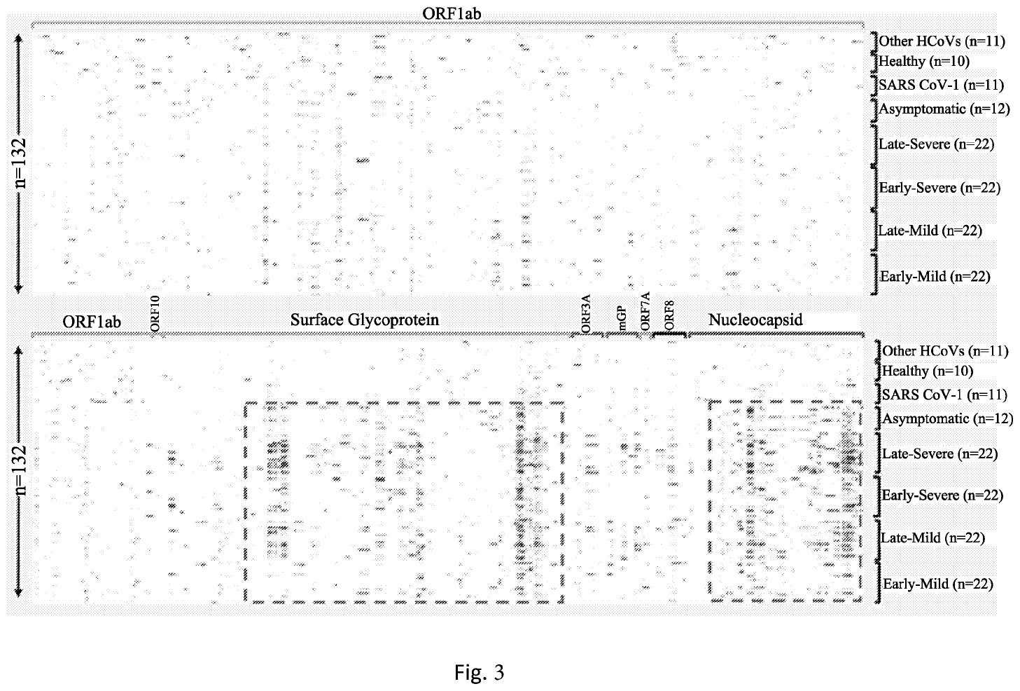

The 981 SARS-CoV-2 peptides with IgG signal intensity >10,000 AU, were used to assemble a heatmap. The signal data was pooled from all the arrays and then reactivity compared across different sample groups. The pooled data were normalized; background corrected, and filtered peptides with immunoreactivity above the 10,000AU to generate the heatmap shown in FIG. 3. Immunoreactive peptides included 566 from ORF1ab, three from ORF10, 243 from surface glycoprotein (S protein), 20 from ORF3a, 20 from membrane glycoprotein (mGP), four from ORF7a, 21 from ORFS, and 104 from nucleocapsid phosphoprotein (N). Peptides from “S” and “N” proteins had higher reactive intensity (higher AU) and rate of reactivity in comparison to peptides from other proteins (FIG. 3). Immunoreactivity was higher in COVID-19 subjects with severe versus mild disease, and in asymptomatic SARS-CoV-2-infected subjects versus subjects with mild disease. Samples from second time point collections were more reactive than first time point collections in subjects with severe, mild and asymptomatic SARSCoV-2 infection (FIG. 3).

The presence of a minimum of three continuous reactive peptides in samples with SARS-CoV-2 infection but non-reactive in control groups, were used to identify SARS-CoV-2 reactive IgG epitopes. Analysis of these 981 peptide sequences led to the identification of 163 epitopes (Table 4). The 29 epitopes with the strongest and most specific reactivity with SARS-CoV-2 are shown in Table 2. Table 2 also indicates the location of each epitope on the SARS-CoV-2 proteome, its length, aa sequence, and the percentages of plasma samples that were immunoreactive in Groups 1-6.

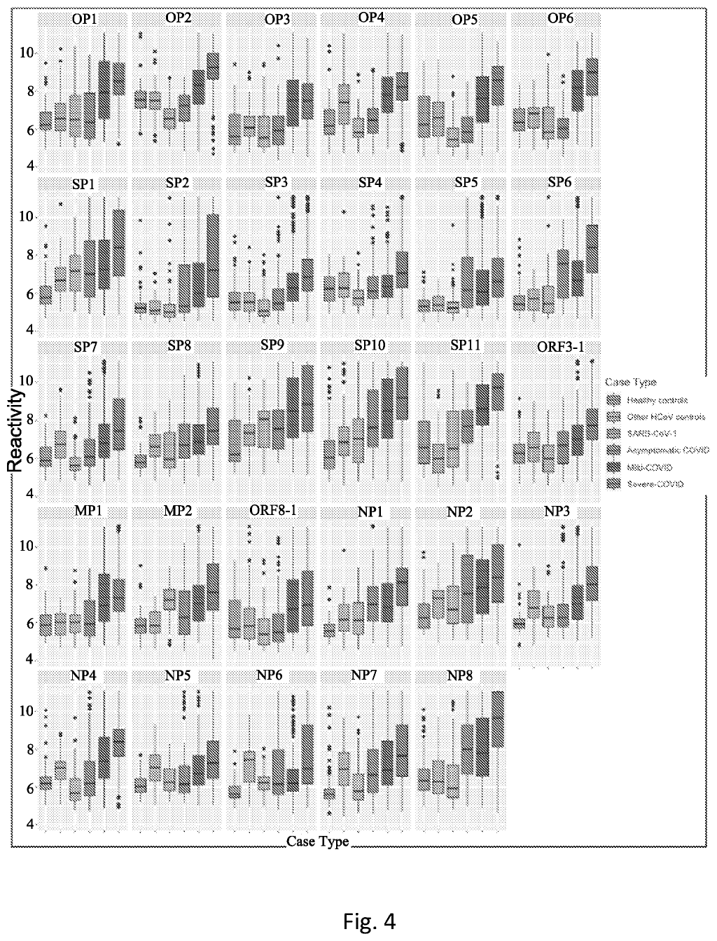

These 29 epitopes included 11 epitopes (37.9%) in S protein (SP1-SP11), 8 (27.5%) epitopes in N protein (NP1-NP8), 6 (20.7%) epitopes in ORF1ab polyprotein (OP1-OP6), 2 (6.9%) in mGP protein (MP1 and MP2), one (3.4%) each from ORF3, and ORF8 proteins. Immunoreactivity was higher in second time point plasma samples (FIG. 4) and in patients with more severe disease. In samples from patients with severe disease, 7-22 epitopes (mean of 13) were reactive in second time point samples (24.8±6.8 days POD) versus 0-15 (mean of 7) epitopes in first time point samples (9.6±3.5 days POD). In patients with mild disease, 3-22 epitopes (mean of eight) were reactive in second time point samples (34.7±8.3 POD) versus 0-12 epitopes (mean of 4) in first time point samples (12.9±5.9 POD). In asymptomatic subjects 1-9 epitopes (mean of four) were reactive at either time point (day of hospitalization and 14.5±4.6 days after the hospitalization). Plasma samples from 19 of 22 patients (86%) with mild disease were reactive with at least 1 of 29 epitopes at the first time point. All 22 (100%) were reactive with at least 3 of 29 epitopes at the second time point. Plasma from 21 of 22 patients (95%) with severe disease were reactive with at least one epitope at first time point. All 22 (100%) were reactive with at least six epitopes at the second time point. All six (100%) asymptomatic cases were reactive with at least 2 of 29 epitopes in first and 3 of 29 in second timepoint collections.

Plasma from none of the asymptomatic cases was reactive with any ORF1ab, ORF3, ORF8, or mGP epitope (Table 4). Plasma from asymptomatic cases were reactive only with S and N epitopes. Plasma collected at both time points from asymptomatic case AS1 was reactive with five epitopes in the S protein (SP1, SP2, SP4, SP5, and SP10) and three epitopes in the N protein (NP2, NP7, and NP8). Case AS1 plasma was reactive with SP9 at only first time point. Plasma from asymptomatic case AS2 was reactive with three epitopes in the S protein (SP7, SP8, and SP10) and five epitopes in the N protein (NP2, NP3, NP4, NPS, and NP8) at both time points. Plasma from asymptomatic cases AS1, AS2, and AS4 were reactive with SP10 and NP8 epitopes at both time points. Plasma from AS3 was reactive with SP10 at both time points and with NP8 only at the second time point. Plasma from AS5 and AS6 were immunoreactive with the NP1 epitope and the epitope NP6, respectively, at both time points. Plasma from mild case M7 was reactive only with the SP10 epitope at first time point but was reactive to 22/29 epitopes at second time point of collection. All mild and severe COVID-19 cases were reactive to a greater number of epitopes at second time points versus the first time point.

The 29 immunoreactive epitopes were mapped to the proteome of SARS-CoV-2 (acc. number MN908947). ORF1ab epitopes (OP1-OP6) were dispersed throughout the protein. Eleven linear epitopes from “S” protein (SP1-SP11) mapped outside of the RBD (Receptor Binding Domain). Four (SP1-SP4) epitopes were located in the SD1/SD2. Four (SP5-SP8) epitopes were located between SD1/SD2 and fusion peptide region. The SP9 epitope overlapped the fusion peptide. The SP10 epitope is located between CD (Connector Domain) region and HR2 (Heptad Repeat 2). The SP11 epitope was located at the beginning of the HR2 region of the S2 subunit of the surface glycoprotein. The SP1 and SP9 peptides identified in this study were recently reported as potentially linked to neutralization (Poh et al. 2020).

Ten of eleven spike epitopes (SP1-SP10) were located in regions of high immunoreactivity reported in a recent pre-print from Li et al. 2020. The SP10 epitope was the most reactive spike epitope in subjects with severe (69% first time point, 96% second timepoint), mild (41% first timepoint, 82% second timepoint), and asymptomatic SARS-CoV-2 (67% either timepoint) infections.

The NP2 and NP8 epitopes were the most reactive N protein epitopes. In severe disease, NP2 reactivity was found in 55% of subjects at the first timepoint, and 82% at the second timepoint. NP8 reactivity was found in 64% of subjects at first timepoint, and 100% at second timepoint. In mild disease, NP2 reactivity was found in 37% of subjects at the first timepoint, and 69% at the second timepoint. NP8 reactivity was found in 28% of subjects at the first timepoint, and 82% at the second timepoint. In asymptomatic cases, reactivity was 59% for either NP2 or NP8. In severe disease SP11 reactivity was found in 46% of subjects at the first time point, and 91% at the second time point. In mild disease SP11 reactivity was found in 28% of subjects at the first time point, and 60% at the second time point. No asymptomatic cases were reactive with SP11. Epitopes for ORF1ab (OP1-OP6) showed reactivity in cases with mild and severe disease at both timepoints but not in asymptomatic infections. Only second timepoint samples from severe and mild diseases were reactive to epitopes for mGP, MP1, and MP2 (37 and 26% for severe disease and 28 and 19% for mild disease).

IgM analysis revealed one linear epitope in the membrane glycoprotein (MADSNGTITVEELKKLLEQWN) (SEQ ID NO: 42), that was reactive in 4/22 (19%) COVID-19 patients with mild disease and 8/22 (37%) patients with severe disease at late time point collection (FIG. 1, Table 3).

Through use of a limited number of plasma samples from subjects with known exposure to other HCoV, IgG epitopes were identified in HKU1 (n=15), NL63 (n=10), OC43 (n=14), 229E (n=5), and SARS-CoV-1 (n=9) (Table 5). More than 90% of samples from patients exposed to SARS-CoV-2 and controls showed a wide range of reactivity to epitopes from seasonal coronaviruses (HKU1, NL63, OC43, and 229E).

In summary, the HCoV peptide array and plasma from 50 patients with asymptomatic, mild, or severe SARS-CoV-2 infection was used to identify immunoreactive IgG epitopes for SARSCoV-2. Immunoreactivity profiles differed with severity of illness and over the time course of infection. Two subjects with a history of SARS-CoV-1 infection had reactivity to two of 29 IgG SARSCoV-2 epitopes. Their plasma was collected in 2004 or 2005; thus, this presumably reflects cross-reactivity due to proteome homology (Lv et al. 2020; Chia et al. 2020). Two healthy controls with immunoreactivity to SP11 (one of 29 epitopes of SARS-CoV-2) may have had an asymptomatic infection with either SARS-CoV-1 or SARS-CoV-2 (Xu et al. 2006; CDC 2003).

TABLE 2
IgG linear epitopes for detection of SARS-CoV-2 infection

| | | | Epitope | AA | AA | Protein name |
|---|---|---|---|---|---|---|
| S. No. | Epitope ID. | Epitope Sequence | length | (start) | (end) | (Acc. MN908947) |
| 1 | OP1 | TAYNGYLTSSSKTP (SEQ ID NO: 1) | 13 | 1,484 | 1,497 | ORF1ab |
| 2 | OP2 | LVPNQPYPNASFDNF (SEQ ID NO: 2) | 14 | 1,914 | 1,928 | polyprotein |
| 3 | OP3 | NRFTTTLNDFNLVAMK (SEQ ID NO: 3) | 15 | 3,484 | 3,499 | (pp1ab) |
| 4 | OP4 | QGLLPPKNSIDAFKLNIK (SEQ ID NO: 4) | 17 | 3,826 | 3,843 | |
| 5 | OP5 | NTCVGSDNVTDFNAIATCDW (SEQ ID NO: 5) | 19 | 5,419 | 5,438 | |
| 6 | OP6 | FYSYATHSDKFTDGV (SEQ ID NO: 6) | 14 | 6,292 | 6,306 | |
| 7 | SP1 | TESNKKFEPFQQFGRDIAD (SEQ ID NO: 7) | 18 | 553 | 571 | Surface |
| 8 | SP2 | TDAVRDPQTLEILDITPCS (SEQ ID NO: 8) | 18 | 573 | 591 | glycoprotein |
| 9 | SP3 | QVAVLYQDVNCTEVPVAIHADQLTPTW (SEQ ID NO: 9) | 26 | 607 | 633 | |
| 10 | SP4 | CASYQTQTNSPRRARSV (SEQ ID NO: 10) | 16 | 671 | 687 | |
| 11 | SP5 | ARSVASQSIIAYTMSLGAENSVA (SEQ ID NO: 11) | 22 | 684 | 706 | |
| 12 | SP6 | RALTGIAVEQDKNTQEVFAQV (SEQ ID NO: 12) | 20 | 765 | 785 | |
| 13 | SP7 | FAQVKQIYKTPPIKDFGGFNF (SEQ ID NO: 13) | 20 | 782 | 802 | |
| 14 | SP8 | NFSQILPDPSKPSKRS (SEQ ID NO: 14) | 15 | 801 | 816 | |
| 15 | SP9 | KRSFIEDLLFNKVT (SEQ ID NO: 15) | 13 | 814 | 827 | |
| 16 | SP10 | PELDSFKEELDKYFKNHTSPDVD (SEQ ID NO: 16) | 22 | 1,143 | 1,165 | |
| 17 | SP11 | SPDVDLGDISGINASVV (SEQ ID NO: 17) | 16 | 1,161 | 1,177 | |
| 18 | ORF3-1 | EHDYQIGGYTEKWESGVKDCVVL (SEQ ID NO: 18) | 22 | 181 | 203 | ORF3a protein |
| 19 | MP1 | CDIKDLPKEITVATS (SEQ ID NO: 19) | 14 | 159 | 173 | Membrane |
| 20 | MP2 | QRVAGDSGFAAYSRY (SEQ ID NO: 20) | 14 | 185 | 199 | glycoprotein |
| 21 | ORF8-1 | GSKSPIQYIDIGNYTVSCLPF (SEQ ID NO: 21) | 20 | 66 | 86 | ORF8 protein |
| 22 | NP1 | ALLLLDRLNQLESKMSG (SEQ ID NO: 22) | 16 | 220 | 236 | Nucleocapsid |
| 23 | NP2 | NQLESKMSGKGQQQQGQTVTKKSA (SEQ ID NO: 23) | 23 | 228 | 251 | phopsphoprotein |
| 24 | NP3 | QTVTKKSAAEASKKPRQK (SEQ ID NO: 24) | 17 | 244 | 261 | |
| 25 | NP4 | RGPEQTQGNFGDQELI (SEQ ID NO: 25) | 15 | 277 | 292 | |
| 26 | NP5 | AYKTFPPTEPKKDKKKK (SEQ ID NO: 26) | 16 | 359 | 375 | |
| 27 | NP6 | KDKKKKADETQALPQR (SEQ ID NO: 27) | 15 | 370 | 385 | |
| 28 | NP7 | ADETQALPQRQKKQQTVTLLPAADL (SEQ ID NO: 28) | 24 | 376 | 400 | |
| 29 | NP8 | TLLPAADLDDFSKQLQQSMSSADS (SEQ ID NO: 29) | 23 | 393 | 416 | |

| | Severe- | Severe- | Mild- | Mild- | Asymptomatic | | | |
|---|---|---|---|---|---|---|---|---|
| | COVID- | COVID- | COVID- | COVID- | COVID- | SARS- | Healthy- | Other- |
| | 19 Early | 19 Late | 19 Early | 19 Late | 19 Late | CoV-1 | Controls | HCoV |
| S. No. | (N = 22) | (N = 22) | (N = 22) | (N = 22) | (N = 12) | (N = 11) | (N = 10) | (N = 11) |
| 1 | 5/22 (23%) | 4/22 (19%) | 5/22 (23%) | 5/22 (23%) | 0/12 (0%) | 1/11 (10%) | 0/10 (0%) | 1/11 (10%) |
| 2 | 8/22 (37%) | 12/22 (55%) | 3/22 (14%) | 3/22 (14%) | 0/12 (0%) | 0/11 (0%) | 0/10 (0%) | 0/11 (0%) |
| 3 | 1/22 (5%) | 1/22 (5%) | 4/22 (19%) | 3/22 (14%) | 0/12 (0%) | 0/11 (0%) | 0/10 (0%) | 0/11 (0%) |
| 4 | 5/22 (23%) | 5/22 (23%) | 4/22 (19%) | 6/22 (28%) | 0/12 (0%) | 0/11 (0%) | 0/10 (0%) | 2/11 (19%) |
| 5 | 5/22 (23%) | 13/22 (60%) | 5/22 (23%) | 6/22 (28%) | 0/12 (0%) | 0/11 (0%) | 0/10 (0%) | 0/11 (0%) |
| 6 | 7/22 (32%) | 11/22 (50%) | 0/22 (0%) | 2/22 (10%) | 0/12 (0%) | 0/11 (0%) | 0/10 (0%) | 0/11 (0%) |
| 7 | 4/22 (19%) | 15/22 (69%) | 1/22 (5%) | 9/22 (41%) | 3/12 (25%) | 0/11 (0%) | 0/10 (0%) | 0/11 (0%) |
| 8 | 6/22 (28%) | 17/22 (78%) | 1/22 (5%) | 11/22 (50%) | 2/12 (17%) | 0/11 (0%) | 0/10 (0%) | 0/11 (0%) |
| 9 | 0/22 (0%) | 9/22 (41%) | 2/22 (10%) | 4/22 (19%) | 0/12 (0%) | 0/11 (0%) | 0/10 (0%) | 0/11 (0%) |
| 10 | 2/22 (10%) | 4/22 (19%) | 0/22 (0%) | 1/22 (5%) | 2/12 (17%) | 0/11 (0%) | 0/10 (0%) | 0/11 (0%) |
| 11 | 2/22 (10%) | 6/22 (28%) | 4/22 (19%) | 4/22 (19%) | 2/12 (17%) | 0/11 (0%) | 0/10 (0%) | 0/11 (0%) |
| 12 | 11/22 (50%) | 14/22 (64%) | 2/22 (10%) | 5/22 (23%) | 2/12 (17%) | 0/11 (0%) | 0/10 (0%) | 0/11 (0%) |
| 13 | 5/22 (23%) | 8/22 (37%) | 1/22 (5%) | 3/22 (14%) | 2/12 (17%) | 0/11 (0%) | 0/10 (0%) | 0/11 (0%) |
| 14 | 1/22 (5%) | 4/22 (19%) | 0/22 (0%) | 4/22 (19%) | 2/12 (17%) | 0/11 (0%) | 0/10 (0%) | 0/11 (0%) |
| 15 | 5/22 (23%) | 8/22 (37%) | 4/22 (19%) | 5/22 (23%) | 1/12 (9%) | 0/11 (0%) | 0/10 (0%) | 0/11 (0%) |
| 16 | 15/22 (69%) | 21/22 (96%) | 9/22 (41%) | 18/22 (82%) | 8/12 (67%) | 1/11 (10%) | 0/10 (0%) | 1/11 (10%) |
| 17 | 10/22 (46%) | 20/22 (91%) | 6/22 (28%) | 13/22 (60%) | 0/12 (0%) | 1/11 (10%) | 2/10 (20%) | 1/11 (10%) |
| 18 | 4/22 (19%) | 7/22 (32%) | 1/22 (5%) | 2/22 (10%) | 0/12 (0%) | 0/11 (0%) | 0/10 (0%) | 0/11 (0%) |
| 19 | 0/22 (0%) | 8/22 (37%) | 0/22 (0%) | 6/22 (28%) | 0/12 (0%) | 0/11 (0%) | 0/10 (0%) | 0/11 (0%) |
| 20 | 0/22 (0%) | 10/22 (46%) | 0/22 (0%) | 4/22 (19%) | 0/12 (0%) | 0/11 (0%) | 0/10 (0%) | 0/11 (0%) |
| 21 | 3/22 (14%) | 12/22 (55%) | 4/22 (19%) | 3/22 (14%) | 0/12 (0%) | 0/11 (0%) | 0/10 (0%) | 1/11 (10%) |
| 22 | 3/22 (14%) | 5/22 (23%) | 2/22 (10%) | 5/22 (23%) | 2/12 (17%) | 0/11 (0%) | 0/10 (0%) | 0/11 (0%) |
| 23 | 12/22 (55%) | 18/22 (82%) | 8/22 (37%) | 15/22 (69%) | 7/12 (59%) | 0/11 (0%) | 0/10 (0%) | 0/11 (0%) |
| 24 | 2/22 (10%) | 7/22 (32%) | 1/22 (5%) | 4/22 (19%) | 2/12 (17%) | 0/11 (0%) | 0/10 (0%) | 0/11 (0%) |
| 25 | 6/22 (28%) | 3/22 (14%) | 2/22 (10%) | 3/22 (14%) | 2/12 (17%) | 0/11 (0%) | 0/10 (0%) | 0/11 (0%) |
| 26 | 2/22 (10%) | 5/22 (23%) | 0/22 (0%) | 2/22 (10%) | 2/12 (17%) | 0/11 (0%) | 0/10 (0%) | 0/11 (0%) |
| 27 | 3/22 (14%) | 7/22 (32%) | 0/22 (0%) | 5/22 (23%) | 3/12 (25%) | 0/11 (0%) | 0/10 (0%) | 0/11 (0%) |
| 28 | 4/22 (19%) | 17/22 (78%) | 4/22 (19%) | 9/22 (41%) | 2/12 (17%) | 0/11 (0%) | 0/10 (0%) | 0/11 (0%) |
| 29 | 14/22 (64%) | 22/22 (100%) | 6/22 (28%) | 18/22 (82%) | 7/12 (59%) | 1/11 (10%) | 0/10 (0%) | 0/11 (0%) |

TABLE 3
IgM linear epitopes for detection of SARS-CoV-2 infection

| | | | | Healthy- | Mild- | Mild- | |
|---|---|---|---|---|---|---|---|
| | | | | Controls_ | COVID_ | COVID_ | Other_ |
| | | | Asymtomatic | China | Early | Late | CoV |
| 1 | LGDELGTDPYEDFQ | SEQ ID | 0/12 (0%) | 0/10 (0%) | 0/22 (0%) | 0/22 (0%) | 1/12 (9%) |
| | | NO: 30 | | | | | |
| 2 | LYIDINGNLHPDSAT | SEQ ID | 0/12 (0%) | 0/10 (0%) | 2/22 (10%) | 0/22 (0%) | 0/12 (0%) |
| | | NO: 31 | | | | | |
| 3 | ATKYLVQQESPFVMM | SEQ ID | 0/12 (0%) | 0/10 (0%) | 0/22 (0%) | 0/22 (0%) | 1/12 (9%) |
| | | NO: 32 | | | | | |
| 4 | IVFDGKSKCEESSA | SEQ ID | 0/12 (0%) | 0/10 (0%) | 1/22 (5%) | 1/22 (5%) | 0/12 (0%) |
| | | NO: 33 | | | | | |
| 5 | LNDFSNSGSDVLYQP | SEQ ID | 0/12 (0%) | 1/10 (10%) | 0/22 (0%) | 0/22 (0%) | 0/12 (0%) |
| | | NO: 34 | | | | | |
| 6 | LSFCAFAVDAAKAY | SEQ ID | 0/12 (0%) | 0/10 (0%) | 0/22 (0%) | 0/22 (0%) | 0/12 (0%) |
| | | NO: 35 | | | | | |
| 7 | YDFGDFIQTTPGSG | SEQ ID | 0/12 (0%) | 1/10 (10%) | 0/22 (0%) | 0/22 (0%) | 0/12 (0%) |
| | | NO: 36 | | | | | |
| 8 | DFIQTTPGSGVPVV | SEQ ID | 0/12 (0%) | 1/10 (10%) | 0/22 (0%) | 0/22 (0%) | 0/12 (0%) |
| | | NO: 37 | | | | | |
| 9 | FLLKYNENGTITDAV | SEQ ID | 0/12 (0%) | 0/10 (0%) | 0/22 (0%) | 0/22 (0%) | 0/12 (0%) |
| | | NO: 38 | | | | | |
| 10 | GVLTESNKKFLPFQ | SEQ ID | 0/12 (0%) | 0/10 (0%) | 0/22 (0%) | 0/22 (0%) | 0/12 (0%) |
| | | NO: 39 | | | | | |
| 11 | YQTQTNSPRRARSVA | SEQ ID | 0/12 (0%) | 0/10 (0%) | 0/22 (0%) | 0/22 (0%) | 0/12 (0%) |
| | | NO: 40 | | | | | |
| 12 | NTQEVFAQVKQIYK | SEQ ID | 0/12 (0%) | 0/10 (0%) | 0/22 (0%) | 0/22 (0%) | 0/12 (0%) |
| | | NO: 41 | | | | | |
| 13 | MADSNGTITVEELKKLLEQWN | SEQ ID | 1/12 (9%) | 0/10 (0%) | 0/22 (0%) | 4/22 (19%) | 0/12 (0%) |
| | | NO: 42 | | | | | |
| 14 | KEPCSSGTYEGNSPFHP | SEQ ID | 0/12 (0%) | 0/10 (0%) | 0/22 (0%) | 1/22 (5%) | 1/12 (9%) |
| | | NO: 43 | | | | | |
| 15 | TLLPAADLDDFSKQ | SEQ ID | 1/12 (9%) | 0/10 (0%) | 0/22 (0%) | 0/22 (0%) | 0/12 (0%) |
| | | NO: 44 | | | | | |
| 16 | AADLDDFSKQLQQS | SEQ ID | 0/12 (0%) | 0/10 (0%) | 0/22 (0%) | 0/22 (0%) | 0/12 (0%) |
| | | NO: 45 | | | | | |

| | | | | | Severe- |
|---|---|---|---|---|---|
| | | | | Severe- | COVID_ |
| | | | SARS_2003 | COVID_Early | Late |
| 1 | LGDELGTDPYEDFQ | SEQ ID NO: 30 | 0/10 (0%) | 0/22 (0%) | 0/22 (0%) |
| 2 | LYIDINGNLHPDSAT | SEQ ID NO: 31 | 0/10 (0%) | 0/22 (0%) | 2/22 (10%) |
| 3 | ATKYLVQQESPFVMM | SEQ ID NO: 32 | 0/10 (0%) | 0/22 (0%) | 0/22 (0%) |
| 4 | IVFDGKSKCEESSA | SEQ ID NO: 33 | 0/10 (0%) | 0/22 (0%) | 0/22 (0%) |
| 5 | LNDFSNSGSDVLYQP | SEQ ID NO: 34 | 0/10 (0%) | 0/22 (0%) | 0/22 (0%) |
| 6 | LSFCAFAVDAAKAY | SEQ ID NO: 35 | 0/10 (0%) | 1/22 (5%) | 0/22 (0%) |
| 7 | YDFGDFIQTTPGSG | SEQ ID NO: 36 | 0/10 (0%) | 0/22 (0%) | 0/22 (0%) |
| 8 | DFIQTTPGSGVPVV | SEQ ID NO: 37 | 0/10 (0%) | 0/22 (0%) | 0/22 (0%) |
| 9 | FLLKYNENGTITDAV | SEQ ID NO: 38 | 0/10 (0%) | 0/22 (0%) | 1/22 (5%) |
| 10 | GVLTESNKKFLPFQ | SEQ ID NO: 39 | 1/10 (10%) | 0/22 (0%) | 0/22 (0%) |
| 11 | YQTQTNSPRRARSVA | SEQ ID NO: 40 | 0/10 (0%) | 0/22 (0%) | 1/22 (5%) |
| 12 | NTQEVFAQVKQIYK | SEQ ID NO: 41 | 0/10 (0%) | 1/22 (5%) | 0/22 (0%) |
| 13 | MADSNGTITVEELKKLLEQWN | SEQ ID NO: 42 | 0/10 (0%) | 1/22 (5%) | 8/22 (37%) |
| 14 | KEPCSSGTYEGNSPFHP | SEQ ID NO: 43 | 0/10 (0%) | 0/22 (0%) | 0/22 (0%) |
| 15 | TLLPAADLDDFSKQ | SEQ ID NO: 44 | 0/10 (0%) | 1/22 (5%) | 1/22 (5%) |
| 16 | AADLDDFSKQLQQS | SEQ ID NO: 45 | 0/10 (0%) | 1/22 (5%) | 0/22 (0%) |

TABLE 4
Additional IgG linear epitopes for detection of SARS-CoV-2 infection.
Sequences (aa) are based on proteome of Severe acute respiratory
syndrome coronavirus 2 isolate Wuhan-Hu-1 (Accession no. MN908947)

| | | Protein Info | Severe- | Severe- | Mild- | Mild- |
|---|---|---|---|---|---|---|
| | | (MN908947_CDS_ | COVID_ | COVID_ | COVID_ | COVID_ |
| Epitope sequence | | proteome) | Early | Late | Early | Late |
| YRKVLLRKNGNKGA | SEQ ID | orf1ab polypro- | 0/22 (0%) | 1/22 | 0/22 | 0/22 |
| | NO: 46 | tein (pp1ab) | | (5%) | (0%) | (0%) |
| LLRKNGNKGAGGHSY | SEQ ID | orf1ab polypro- | 0/22 (0%) | 0/22 | 0/22 | 0/22 |
| | NO: 47 | tein (pp1ab) | | (0%) | (0%) | (0%) |
| GNKGAGGHSYGADLKSF | SEQ ID | orf1ab polypro- | 0/22 (0%) | 0/22 | 0/22 | 0/22 |
| DL | NO: 48 | tein (pp1ab) | | (0%) | (0%) | (0%) |
| DELGTDPYEDFQEN | SEQ ID | orf1ab polypro- | 0/22 (0%) | 0/22 | 0/22 | 0/22 |
| | NO: 49 | tein (pp1ab) | | (0%) | (0%) | (0%) |
| WNTKHSSGVTRELM | SEQ ID | orf1ab polypro- | 0/22 (0%) | 0/22 | 1/22 | 1/22 |
| | NO: 50 | tein (pp1ab) | | (0%) | (5%) | (5%) |
| GVYCCREHEHEIAW | SEQ ID | orf1ab polypro- | 0/22 (0%) | 0/22 | 0/22 | 0/22 |
| | NO: 51 | tein (pp1ab) | | (0%) | (0%) | (0%) |
| HEIAWYTERSEKSY | SEQ ID | orf1ab polypro- | 0/22 (0%) | 0/22 | 0/22 | 0/22 |
| | NO: 52 | tein (pp1ab) | | (0%) | (0%) | (0%) |
| SYELQTPFEIKLAK | SEQ ID | orf1ab polypro- | 1/22 (5%) | 1/22 | 0/22 | 0/22 |
| | NO: 53 | tein (pp1ab) | | (5%) | (0%) | (0%) |
| AKKFDTFNGECPNF | SEQ ID | orf1ab polypro- | 0/22 (0%) | 0/22 | 0/22 | 0/22 |
| | NO: 54 | tein (pp1ab) | | (0%) | (0%) | (0%) |
| CDHCGETSWQTGDF | SEQ ID | orf1ab polypro- | 0/22 (0%) | 0/22 | 1/22 | 1/22 |
| | NO: 55 | tein (pp1ab) | | (0%) | (5%) | (5%) |
| LTNIFGTVYEKLKPVLD | SEQ ID | orf1ab polypro- | 0/22 (0%) | 0/22 | 0/22 | 0/22 |
| | NO: 56 | tein (pp1ab) | | (0%) | (0%) | (0%) |
| GVEFLRDGWEIVKF | SEQ ID | orf1ab polypro- | 1/22 (5%) | 0/22 | 0/22 | 0/22 |
| | NO: 57 | tein (pp1ab) | | (0%) | (0%) | (0%) |
| TGDLQPLEQPTSEAVEAP | SEQ ID | orf1ab polypro- | 0/22 (0%) | 0/22 | 0/22 | 0/22 |
| LV | NO: 58 | tein (pp1ab) | | (0%) | (0%) | (0%) |
| KGGAPTKVTFGDDTVIEV | SEQ ID | orf1ab polypro- | 0/22 (0%) | 0/22 | 0/22 | 0/22 |
| | NO: 59 | tein (pp1ab) | | (0%) | (0%) | (0%) |
| NITFELDERIDKVL | SEQ ID | orf1ab polypro- | 0/22 (0%) | 1/22 | 0/22 | 0/22 |
| | NO: 60 | tein (pp1ab) | | (5%) | (0%) | (0%) |
| EFACVVADAVIKTLQPVS | SEQ ID | orf1ab polypro- | 1/22 (5%) | 1/22 | 0/22 | 1/22 |
| | NO: 61 | tein (pp1ab) | | (5%) | (0%) | (5%) |
| NKGEDIQLLKSAYENF | SEQ ID | orf1ab polypro- | 0/22 (0%) | 1/22 | 0/22 | 0/22 |
| | NO: 62 | tein (pp1ab) | | (5%) | (0%) | (0%) |
| YDKLVSSFLEMKSEK | SEQ ID | orf1ab polypro- | 0/22 (0%) | 1/22 | 0/22 | 0/22 |
| | NO: 63 | tein (pp1ab) | | (5%) | (0%) | (0%) |
| AFYILPSIISNEKQEI | SEQ ID | orf1ab polypro- | 0/22 (0%) | 1/22 | 0/22 | 0/22 |
| | NO: 64 | tein (pp1ab) | | (5%) | (0%) | (0%) |
| LGYVTHGLNLEEAA | SEQ ID | orf1ab polypro- | 1/22 (5%) | 2/22 | 0/22 | 0/22 |
| | NO: 65 | tein (pp1ab) | | (10%) | (0%) | (0%) |
| THGLNLEEAARYMRS | SEQ ID | orf1ab polypro- | 0/22 (0%) | 1/22 | 0/22 | 1/22 |
| | NO: 66 | tein (pp1ab) | | (5%) | (0%) | (5%) |
| TAYNGYLTSSSKTP | SEQ ID | orf1ab polypro- | 5/22 (23%) | 4/22 | 5/22 | 5/22 |
| | NO: 67 | tein (pp1ab) | | (19%) | (23%) | (23%) |
| GEVITFDNLKTLLS | SEQ ID | orf1ab polypro- | 0/22 (0%) | 1/22 | 1/22 | 1/22 |
| | NO: 68 | tein (pp1ab) | | (5%) | (5%) | (5%) |
| GRYMSALNHTKKWKYPQ | SEQ ID | orf1ab polypro- | 0/22 (0%) | 1/22 | 0/22 | 0/22 |
| V | NO: 69 | tein (pp1ab) | | (5%) | (0%) | (0%) |
| TVGELGDVRETMSYL | SEQ ID | orf1ab polypro- | 1/22 (5%) | 0/22 | 1/22 | 0/22 |
| | NO: 70 | tein (pp1ab) | | (0%) | (5%) | (0%) |
| MGTLSYEQFKKGVQIPC | SEQ ID | orf1ab polypro- | 0/22 (0%) | 1/22 | 0/22 | 0/22 |
| | NO: 71 | tein (pp1ab) | | (5%) | (0%) | (0%) |
| FTCASEYTGNYQCGHY | SEQ ID | orf1ab polypro- | 2/22 (10%) | 2/22 | 3/22 | 3/22 |
| | NO: 72 | tein (pp1ab) | | (10%) | (14%) | (14%) |
| YQCGHYKHITSKET | SEQ ID | orf1ab polypro- | 0/22 (0%) | 0/22 | 0/22 | 0/22 |
| | NO: 73 | tein (pp1ab) | | (0%) | (0%) | (0%) |
| LVPNQPYPNASFDNF | SEQ ID | orf1ab polypro- | 8/22 (37%) | 12/22 | 3/22 | 3/22 |
| | NO: 74 | tein (pp1ab) | | (55%) | (14%) | (14%) |
| FPDLNGDVVAIDYK | SEQ ID | orf1ab polypro- | 1/22 (5%) | 1/22 | 0/22 | 0/22 |
| | NO: 75 | tein (pp1ab) | | (5%) | (0%) | (0%) |
| NSFDVLKSEDAQGMDNL | SEQ ID | orf1ab polypro- | 0/22 (0%) | 0/22 | 0/22 | 0/22 |
| | NO: 76 | tein (pp1ab) | | (0%) | (0%) | (0%) |
| SEEVVENPTIQKDVLECN | SEQ ID | orf1ab polypro- | 0/22 (0%) | 2/22 | 0/22 | 1/22 |
| | NO: 77 | tein (pp1ab) | | (10%) | (0%) | (5%) |
| GLAAVNSVPWDTIA | SEQ ID | orf1ab polypro- | 0/22 (0%) | 0/22 | 1/22 | 0/22 |
| | NO: 78 | tein (pp1ab) | | (0%) | (5%) | (0%) |
| TIANYAKPFLNKVVSTTT | SEQ ID | orf1ab polypro- | 1/22 (5%) | 2/22 | 1/22 | 1/22 |
| | NO: 79 | tein (pp1ab) | | (10%) | (5%) | (5%) |
| FLNKVVSTTTNIVTRC | SEQ ID | orf1ab polypro- | 0/22 (0%) | 0/22 | 0/22 | 0/22 |
| | NO: 80 | tein (pp1ab) | | (0%) | (0%) | (0%) |
| VKMFDAYVNTFSST | SEQ ID | orf1ab polypro- | 2/22 (10%) | 3/22 | 1/22 | 0/22 |
| | NO: 81 | tein (pp1ab) | | (14%) | (5%) | (0%) |
| FSSTFNVPMEKLKT | SEQ ID | orf1ab polypro- | 0/22 (0%) | 0/22 | 0/22 | 0/22 |
| | NO: 82 | tein (pp1ab) | | (0%) | (0%) | (0%) |
| TLVATAEAELAKNVSL | SEQ ID | orf1ab polypro- | 0/22 (0%) | 0/22 | 0/22 | 0/22 |
| | NO: 83 | tein (pp1ab) | | (0%) | (0%) | (0%) |
| SAAKKNNLPFKLTCA | SEQ ID | orf1ab polypro- | 0/22 (0%) | 1/22 | 0/22 | 0/22 |
| | NO: 84 | tein (pp1ab) | | (5%) | (0%) | (0%) |
| VHVMSKHTDFSSEIIG | SEQ ID | orf1ab polypro- | 1/22 (5%) | 1/22 | 0/22 | 0/22 |
| | NO: 85 | tein (pp1ab) | | (5%) | (0%) | (0%) |
| DFDTWFSQRGGSYT | SEQ ID | orf1ab polypro- | 0/22 (0%) | 0/22 | 0/22 | 0/22 |
| | NO: 86 | tein (pp1ab) | | (0%) | (0%) | (0%) |
| TFDSEYCRHGTCER | SEQ ID | orf1ab polypro- | 0/22 (0%) | 0/22 | 0/22 | 0/22 |
| | NO: 87 | tein (pp1ab) | | (0%) | (0%) | (0%) |
| DVLYQPPQTSITSAVL | SEQ ID | orf1ab polypro- | 2/22 (10%) | 1/22 | 1/22 | 0/22 |
| | NO: 88 | tein (pp1ab) | | (5%) | (5%) | (0%) |
| NRFTTTLNDFNLVAMK | SEQ ID | orf1ab polypro- | 1/22 (5%) | 1/22 | 4/22 | 3/22 |
| | NO: 89 | tein (pp1ab) | | (5%) | (19%) | (14%) |
| LTQDHVDILGPLSA | SEQ ID | orf1ab polypro- | 0/22 (0%) | 0/22 | 0/22 | 1/22 |
| | NO: 90 | tein (pp1ab) | | (0%) | (0%) | (5%) |
| GMNGRTILGSALLED | SEQ ID | orf1ab polypro- | 0/22 (0%) | 0/22 | 0/22 | 1/22 |
| | NO: 91 | tein (pp1ab) | | (0%) | (0%) | (5%) |
| QGLLPPKNSIDAFKLNIK | SEQ ID | orf1ab polypro- | 5/22 (23%) | 5/22 | 4/22 | 6/22 |
| | NO: 92 | tein (pp1ab) | | (23%) | (19%) | (28%) |
| KLKKSLNVAKSEFDRD | SEQ ID | orf1ab polypro- | 0/22 (0%) | 0/22 | 0/22 | 0/22 |
| | NO: 93 | tein (pp1ab) | | (0%) | (0%) | (0%) |
| LRANSAVKLQNNELS | SEQ ID | orf1ab polypro- | 0/22 (0%) | 0/22 | 0/22 | 0/22 |
| | NO: 94 | tein (pp1ab) | | (0%) | (0%) | (0%) |
| AVKLQNNELSPVALR | SEQ ID | orf1ab polypro- | 1/22 (5%) | 1/22 | 0/22 | 0/22 |
| | NO: 95 | tein (pp1ab) | | (5%) | (0%) | (0%) |
| CAAGTTQTACTDDN | SEQ ID | orf1ab polypro- | 0/22 (0%) | 0/22 | 1/22 | 1/22 |
| | NO: 96 | tein (pp1ab) | | (0%) | (5%) | (5%) |
| LALLSDLQDLKWARF | SEQ ID | orf1ab polypro- | 0/22 (0%) | 1/22 | 0/22 | 0/22 |
| | NO: 97 | tein (pp1ab) | | (5%) | (0%) | (0%) |
| ATVRLQAGNATEVPA | SEQ ID | orf1ab polypro- | 0/22 (0%) | 0/22 | 0/22 | 0/22 |
| | NO: 98 | tein (pp1ab) | | (0%) | (0%) | (0%) |
| VKMLCTHTGTGQAITV | SEQ ID | orf1ab polypro- | 0/22 (0%) | 0/22 | 2/22 | 2/22 |
| | NO: 99 | tein (pp1ab) | | (0%) | (10%) | (10%) |
| KYVQIPTTCANDPV | SEQ ID | orf1ab polypro- | 0/22 (0%) | 0/22 | 0/22 | 0/22 |
| | NO: 100 | tein (pp1ab) | | (0%) | (0%) | (0%) |
| RVCGVSAARLTPCGT | SEQ ID | orf1ab polypro- | 2/22 (10%) | 1/22 | 0/22 | 0/22 |
| | NO: 101 | tein (pp1ab) | | (5%) | (0%) | (0%) |
| AVAKHDFFKFRIDG | SEQ ID | orf1ab polypro- | 0/22 (0%) | 0/22 | 0/22 | 0/22 |
| | NO: 102 | tein (pp1ab) | | (0%) | (0%) | (0%) |
| GDFIQTTPGSGVPV | SEQ ID | orf1ab polypro- | 0/22 (0%) | 0/22 | 0/22 | 0/22 |
| | NO: 103 | tein (pp1ab) | | (0%) | (0%) | (0%) |
| QTYHPNCVNCLDDR | SEQ ID | orf1ab polypro- | 0/22 (0%) | 1/22 | 1/22 | 2/22 |
| | NO: 104 | tein (pp1ab) | | (5%) | (5%) | (10%) |
| ALTNNVAFQTVKPGNF | SEQ ID | orf1ab polypro- | 1/22 (5%) | 0/22 | 0/22 | 0/22 |
| | NO: 105 | tein (pp1ab) | | (0%) | (0%) | (0%) |
| GFFKEGSSVELKHF | SEQ ID | orf1ab polypro- | 0/22 (0%) | 1/22 | 0/22 | 0/22 |
| | NO: 106 | tein (pp1ab) | | (5%) | (0%) | (0%) |
| QLLFVVEVVDKYFDCYD | SEQ ID | orf1ab polypro- | 0/22 (0%) | 0/22 | 1/22 | 1/22 |
| GGC | NO: 107 | tein (pp1ab) | | (0%) | (5%) | (5%) |
| SLYVKPGGTSSGDATTAY | SEQ ID | orf1ab polypro- | 0/22 (0%) | 0/22 | 0/22 | 0/22 |
| A | NO: 108 | tein (pp1ab) | | (0%) | (0%) | (0%) |
| NRDVDTDFVNEFYAYL | SEQ ID | orf1ab polypro- | 0/22 (0%) | 1/22 | 2/22 | 2/22 |
| | NO: 109 | tein (pp1ab) | | (5%) | (10%) | (10%) |
| SQGLVASIKNFKSV | SEQ ID | orf1ab polypro- | 0/22 (0%) | 0/22 | 0/22 | 0/22 |
| | NO: 110 | tein (pp1ab) | | (0%) | (0%) | (0%) |
| QHTMLVKQGDDYVY | SEQ ID | orf1ab polypro- | 0/22 (0%) | 1/22 | 0/22 | 0/22 |
| | NO: 111 | tein (pp1ab) | | (5%) | (0%) | (0%) |
| VDDIVKTDGTLMIE | SEQ ID | orf1ab polypro- | 0/22 (0%) | 1/22 | 0/22 | 0/22 |
| | NO: 112 | tein (pp1ab) | | (5%) | (0%) | (0%) |
| AIDAYPLTKHPNQE | SEQ ID | orf1ab polypro- | 0/22 (0%) | 0/22 | 0/22 | 0/22 |
| | NO: 113 | tein (pp1ab) | | (0%) | (0%) | (0%) |
| GCDVTDVTQLYLGG | SEQ ID | orf1ab polypro- | 1/22 (5%) | 1/22 | 0/22 | 0/22 |
| | NO: 114 | tein (pp1ab) | | (5%) | (0%) | (0%) |
| GLYKNTCVGSDNVT | SEQ ID | orf1ab polypro- | 1/22 (5%) | 1/22 | 0/22 | 0/22 |
| | NO: 115 | tein (pp1ab) | | (5%) | (0%) | (0%) |
| NTCVGSDNVTDFNAIATC | SEQ ID | orf1ab polypro- | 5/22 (23%) | 13/22 | 5/22 | 6/22 |
| DW | NO: 116 | tein (pp1ab) | | (60%) | (23%) | (28%) |
| YQKVGMQKYSTLQG | SEQ ID | orf1ab polypro- | 0/22 (0%) | 0/22 | 0/22 | 0/22 |
| | NO: 117 | tein (pp1ab) | | (0%) | (0%) | (0%) |
| STLQGPPGTGKSHF | SEQ ID | orf1ab polypro- | 2/22 (10%) | 0/22 | 1/22 | 2/22 |
| | NO: 118 | tein (pp1ab) | | (0%) | (5%) | (10%) |
| ARARVECFDKFKVNS | SEQ ID | orf1ab polypro- | 0/22 (0%) | 0/22 | 1/22 | 1/22 |
| | NO: 119 | tein (pp1ab) | | (0%) | (5%) | (5%) |
| DPAQLPAPRTLLTK | SEQ ID | orf1ab polypro- | 0/22 (0%) | 0/22 | 0/22 | 0/22 |
| | NO: 120 | tein (pp1ab) | | (0%) | (0%) | (0%) |
| PRTLLTKGTLEPEY | SEQ ID | orf1ab polypro- | 2/22 (10%) | 2/22 | 0/22 | 0/22 |
| | NO: 121 | tein (pp1ab) | | (10%) | (0%) | (0%) |
| IFTQTTETAHSCNV | SEQ ID | orf1ab polypro- | 1/22 (5%) | 1/22 | 0/22 | 1/22 |
| | NO: 122 | tein (pp1ab) | | (5%) | (0%) | (5%) |
| GCHATREAVGTNLP | SEQ ID | orf1ab polypro- | 0/22 (0%) | 0/22 | 1/22 | 1/22 |
| | NO: 123 | tein (pp1ab) | | (0%) | (5%) | (5%) |
| VQMLSDTLKNLSDR | SEQ ID | orf1ab polypro- | 1/22 (5%) | 1/22 | 0/22 | 0/22 |
| | NO: 124 | tein (pp1ab) | | (5%) | (0%) | (0%) |
| FDYVYNPFMIDVQQWG | SEQ ID | orf1ab polypro- | 0/22 (0%) | 0/22 | 0/22 | 0/22 |
| | NO: 125 | tein (pp1ab) | | (0%) | (0%) | (0%) |
| MIDVQQWGFTGNLQ | SEQ ID | orf1ab polypro- | 1/22 (5%) | 1/22 | 0/22 | 0/22 |
| | NO: 126 | tein (pp1ab) | | (5%) | (0%) | (0%) |
| TGNLQSNHDLYCQVH | SEQ ID | orf1ab polypro- | 0/22 (0%) | 1/22 | 0/22 | 0/22 |
| | NO: 127 | tein (pp1ab) | | (5%) | (0%) | (0%) |
| LADKFPVLHDIGNPKAIK | SEQ ID | orf1ab polypro- | 0/22 (0%) | 0/22 | 0/22 | 0/22 |
| CVP | NO: 128 | tein (pp1ab) | | (0%) | (0%) | (0%) |
| FYSYATHSDKFTDGV | SEQ ID | orf1ab polypro- | 7/22 (32%) | 11/22 | 0/22 | 2/22 |
| | NO: 129 | tein (pp1ab) | | (50%) | (0%) | (10%) |
| FDTRVLSNLNLPGCD | SEQ ID | orf1ab polypro- | 0/22 (0%) | 0/22 | 0/22 | 0/22 |
| | NO: 130 | tein (pp1ab) | | (0%) | (0%) | (0%) |
| QVVSDIDYVPLKSATC | SEQ ID | orf1ab polypro- | 3/22 (14%) | 3/22 | 1/22 | 1/22 |
| | NO: 131 | tein (pp1ab) | | (14%) | (5%) | (5%) |
| PSVGPKQASLNGVTL | SEQ ID | orf1ab polypro- | 1/22 (5%) | 0/22 | 0/22 | 0/22 |
| | NO: 132 | tein (pp1ab) | | (0%) | (0%) | (0%) |
| GVVQQLPETYFTQSRN | SEQ ID | orf1ab polypro- | 1/22 (5%) | 1/22 | 0/22 | 0/22 |
| | NO: 133 | tein (pp1ab) | | (5%) | (0%) | (0%) |
| AMDEFIERYKLEGY | SEQ ID | orf1ab polypro- | 3/22 (14%) | 6/22 | 0/22 | 1/22 |
| | NO: 134 | tein (pp1ab) | | (28%) | (0%) | (5%) |
| LAKRFKESPFELED | SEQ ID | orf1ab polypro- | 0/22 (0%) | 0/22 | 0/22 | 0/22 |
| | NO: 135 | tein (pp1ab) | | (0%) | (0%) | (0%) |
| FITDAQTGSSKCVCSVID | SEQ ID | orf1ab polypro- | 0/22 (0%) | 0/22 | 0/22 | 0/22 |
| | NO: 136 | tein (pp1ab) | | (0%) | (0%) | (0%) |
| IKSQDLSVVSKVVKV | SEQ ID | orf1ab polypro- | 0/22 (0%) | 0/22 | 0/22 | 0/22 |
| | NO: 137 | tein (pp1ab) | | (0%) | (0%) | (0%) |
| YPKLQSSQAWQPGV | SEQ ID | orf1ab polypro- | 0/22 (0%) | 0/22 | 1/22 | 0/22 |
| | NO: 138 | tein (pp1ab) | | (0%) | (5%) | (0%) |
| QRMLLEKCDLQNYG | SEQ ID | orf1ab polypro- | 0/22 (0%) | 0/22 | 0/22 | 0/22 |
| | NO: 139 | tein (pp1ab) | | (0%) | (0%) | (0%) |
| HFGAGSDKGVAPGTA | SEQ ID | orf1ab polypro- | 0/22 (0%) | 0/22 | 0/22 | 0/22 |
| | NO: 140 | tein (pp1ab) | | (0%) | (0%) | (0%) |
| ITEHSWNADLYKLM | SEQ ID | orf1ab polypro- | 0/22 (0%) | 0/22 | 1/22 | 1/22 |
| | NO: 141 | tein (pp1ab) | | (0%) | (5%) | (5%) |
| LSSYSLFDMSKFPLKL | SEQ ID | orf1ab polypro- | 2/22 (10%) | 3/22 | 0/22 | 0/22 |
| | NO: 142 | tein (pp1ab) | | (14%) | (0%) | (0%) |
| SLLLCRMNSRNYIA | SEQ ID | ORF10 protein | 1/22 (5%) | 1/22 | 0/22 | 0/22 |
| | NO: 143 | | | (5%) | (0%) | (0%) |
| QCVNLTTRTQLPPA | SEQ ID | surface | 0/22 (0%) | 0/22 | 0/22 | 0/22 |
| | NO: 144 | glycoprotein | | (0%) | (0%) | (0%) |
| TQLPPAYTNSFTRGVYYP | SEQ ID | surface | 4/22 (19%) | 5/22 | 2/22 | 2/22 |
| D | NO: 145 | glycoprotein | | (23%) | (10%) | (10%) |
| FHAIHVSGTNGTKRF | SEQ ID | surface | 0/22 (0%) | 0/22 | 1/22 | 0/22 |
| | NO: 146 | glycoprotein | | (0%) | (5%) | (0%) |
| NLVRDLPQGFSALEPLV | SEQ ID | surface | 0/22 (0%) | 1/22 | 1/22 | 1/22 |
| | NO: 147 | glycoprotein | | (5%) | (5%) | (5%) |
| LALHRSYLTPGDSSSGWT | SEQ ID | surface | 1/22 (5%) | 3/22 | 0/22 | 2/22 |
| | NO: 148 | glycoprotein | | (14%) | (0%) | (10%) |
| KYNENGTITDAVDCALDP | SEQ ID | surface | 1/22 (5%) | 1/22 | 1/22 | 1/22 |
| LS | NO: 149 | glycoprotein | | (5%) | (5%) | (5%) |
| IYQAGSTPCNGVEGFNCY | SEQ ID | surface | 0/22 (0%) | 0/22 | 0/22 | 0/22 |
| | NO: 150 | glycoprotein | | (0%) | (0%) | (0%) |
| VLSFELLHAPATVC | SEQ ID | surface | 1/22 (5%) | 0/22 | 0/22 | 0/22 |
| | NO: 151 | glycoprotein | | (0%) | (0%) | (0%) |
| LLHAPATVCGPKKST | SEQ ID | surface | 0/22 (0%) | 2/22 | 0/22 | 1/22 |
| | NO: 152 | glycoprotein | | (10%) | (0%) | (5%) |
| VKNKCVNFNFNGLT | SEQ ID | surface | 0/22 (0%) | 0/22 | 0/22 | 0/22 |
| | NO: 153 | glycoprotein | | (0%) | (0%) | (0%) |
| TESNKKFLPFQQFGRDIA | SEQ ID | surface | 4/22 (19%) | 15/22 | 1/22 | 9/22 |
| D | NO: 154 | glycoprotein | | (69%) | (5%) | (41%) |
| TDAVRDPQTLEILDITPC | SEQ ID | surface | 6/22 (28%) | 17/22 | 1/22 | 11/22 |
| S | NO: 155 | glycoprotein | | (78%) | (5%) | (50%) |
| QVAVLYQDVNCTEVPVAI | SEQ ID | surface | 0/22 (0%) | 9/22 | 2/22 | 4/22 |
| HADQLTPTW | NO: 156 | glycoprotein | | (41%) | (10%) | (19%) |
| IHADQLTPTWRVYSTGSN | SEQ ID | surface | 0/22 (0%) | 3/22 | 0/22 | 2/22 |
| | NO: 157 | glycoprotein | | (14%) | (0%) | (10%) |
| YSTGSNVFQTRAGCL | SEQ ID | surface | 2/22 (10%) | 1/22 | 0/22 | 0/22 |
| | NO: 158 | glycoprotein | | (5%) | (0%) | (0%) |
| CASYQTQTNSPRRARSV | SEQ ID | surface | 2/22 (10%) | 4/22 | 0/22 | 1/22 |
| | NO: 159 | glycoprotein | | (19%) | (0%) | (5%) |
| ARSVASQSIIAYTMSLGA | SEQ ID | surface | 2/22 (10%) | 6/22 | 4/22 | 4/22 |
| ENSVA | NO: 160 | glycoprotein | | (28%) | (19%) | (19%) |
| PVSMTKTSVDCTMY | SEQ ID | surface | 0/22 (0%) | 0/22 | 0/22 | 0/22 |
| | NO: 161 | glycoprotein | | (0%) | (0%) | (0%) |
| RALTGIAVEQDKNTQEVF | SEQ ID | surface | 11/22 (50%) | 14/22 | 2/22 | 5/22 |
| AQV | NO: 162 | glycoprotein | | (64%) | (10%) | (23%) |
| FAQVKQIYKTPPIKDFGG | SEQ ID | surface | 5/22 (23%) | 8/22 | 1/22 | 3/22 |
| FNF | NO: 163 | glycoprotein | | (37%) | (5%) | (14%) |
| NFSQILPDPSKPSKRS | SEQ ID | surface | 1/22 (5%) | 4/22 | 0/22 | 4/22 |
| | NO: 164 | glycoprotein | | (19%) | (0%) | (19%) |
| KRSFIEDLLFNKVT | SEQ ID | surface | 5/22 (23%) | 8/22 | 4/22 | 5/22 |
| | NO: 165 | glycoprotein | | (37%) | (19%) | (23%) |
| EDLLFNKVTLADAGF | SEQ ID | surface | 1/22 (5%) | 1/22 | 1/22 | 0/22 |
| | NO: 166 | glycoprotein | | (5%) | (5%) | (0%) |
| YGDCLGDIAARDLIC | SEQ ID | surface | 1/22 (5%) | 3/22 | 1/22 | 0/22 |
| | NO: 167 | glycoprotein | | (14%) | (5%) | (0%) |
| ICAQKFNGLTVLPPLL | SEQ ID | surface | 0/22 (0%) | 0/22 | 0/22 | 0/22 |
| | NO: 168 | glycoprotein | | (0%) | (0%) | (0%) |
| FGAGAALQIPFAMQM | SEQ ID | surface | 0/22 (0%) | 0/22 | 0/22 | 0/22 |
| | NO: 169 | glycoprotein | | (0%) | (0%) | (0%) |
| NGIGVTQNVLYENQK | SEQ ID | surface | 1/22 (5%) | 0/22 | 1/22 | 1/22 |
| | NO: 170 | glycoprotein | | (0%) | (5%) | (5%) |
| IGKIQDSLSSTASA | SEQ ID | surface | 0/22 (0%) | 1/22 | 1/22 | 0/22 |
| | NO: 171 | glycoprotein | | (5%) | (5%) | (0%) |
| VLNDILSRLDKVEAE | SEQ ID | surface | 0/22 (0%) | 2/22 | 0/22 | 0/22 |
| | NO: 172 | glycoprotein | | (10%) | (0%) | (0%) |
| LSRLDKVEAEVQID | SEQ ID | surface | 0/22 (0%) | 0/22 | 0/22 | 0/22 |
| | NO: 173 | glycoprotein | | (0%) | (0%) | (0%) |
| AICHDGKAHFPREGVF | SEQ ID | surface | 0/22 (0%) | 0/22 | 0/22 | 0/22 |
| | NO: 174 | glycoprotein | | (0%) | (0%) | (0%) |
| HWFVTQRNFYEPQIITT | SEQ ID | surface | 0/22 (0%) | 0/22 | 1/22 | 1/22 |
| | NO: 175 | glycoprotein | | (0%) | (5%) | (5%) |
| VIGIVNNTVYDPLQPEL | SEQ ID | surface | 2/22 (10%) | 2/22 | 0/22 | 1/22 |
| | NO: 176 | glycoprotein | | (10%) | (0%) | (5%) |
| TVYDPLQPELDSFK | SEQ ID | surface | 1/22 (5%) | 1/22 | 0/22 | 0/22 |
| | NO: 177 | glycoprotein | | (5%) | (0%) | (0%) |
| PELDSFKEELDKYFKNHT | SEQ ID | surface | 15/22 (69%) | 21/22 | 9/22 | 18/22 |
| SPDVD | NO: 178 | glycoprotein | | (96%) | (41%) | (82%) |
| SPDVDLGDISGINASVV | SEQ ID | surface | 10/22 (46%) | 20/22 | 6/22 | 13/22 |
| | NO: 179 | glycoprotein | | (91%) | (28%) | (60%) |
| EIDRLNEVAKNLNESL | SEQ ID | surface | 0/22 (0%) | 5/22 | 0/22 | 1/22 |
| | NO: 180 | glycoprotein | | (23%) | (0%) | (5%) |
| EVAKNLNESLIDLQ | SEQ ID | surface | 1/22 (5%) | 1/22 | 2/22 | 1/22 |
| | NO: 181 | glycoprotein | | (5%) | (10%) | (5%) |
| SCGSCCKFDEDDSE | SEQ ID | surface | 0/22 (0%) | 0/22 | 0/22 | 0/22 |
| | NO: 182 | glycoprotein | | (0%) | (0%) | (0%) |
| FDEDDSEPVLKGVKLH | SEQ ID | surface | 0/22 (0%) | 3/22 | 0/22 | 1/22 |
| | NO: 183 | glycoprotein | | (14%) | (0%) | (5%) |
| TIGTVTLKQGEIKDAT | SEQ ID | ORF3a protein | 0/22 (0%) | 2/22 | 0/22 | 1/22 |
| | NO: 184 | | | (10%) | (0%) | (5%) |
| EHDYQIGGYTEKWESGV | SEQ ID | ORF3a protein | 4/22 (19%) | 7/22 | 1/22 | 2/22 |
| KDCVVL | NO: 185 | | | (32%) | (5%) | (10%) |
| MADSNGTITVEELK | SEQ ID | membrane | 0/22 (0%) | 0/22 | 2/22 | 2/22 |
| | NO: 186 | glycoprotein | | (0%) | (10%) | (10%) |
| RSMWSFNPETNILLNV | SEQ ID | membrane | 0/22 (0%) | 1/22 | 0/22 | 0/22 |
| | NO: 187 | glycoprotein | | (5%) | (0%) | (0%) |
| CDIKDLPKEITVATS | SEQ ID | membrane | 0/22 (0%) | 8/22 | 0/22 | 6/22 |
| | NO: 188 | glycoprotein | | (37%) | (0%) | (28%) |
| KLGASQRVAGDSGFA | SEQ ID | membrane | 0/22 (0%) | 1/22 | 0/22 | 0/22 |
| | NO: 189 | glycoprotein | | (5%) | (0%) | (0%) |
| QRVAGDSGFAAYSRY | SEQ ID | membrane | 0/22 (0%) | 10/22 | 0/22 | 4/22 |
| | NO: 190 | glycoprotein | | (46%) | (0%) | (19%) |
| SSGTYEGNSPFHPLA | SEQ ID | ORF7a protein | 1/22 (5%) | 1/22 | 1/22 | 1/22 |
| | NO: 191 | | | (5%) | (5%) | (5%) |
| LIELCVDEAGSKSPIQYI | SEQ ID | ORF8 protein | 0/22 (0%) | 3/22 | 2/22 | 1/22 |
| D | NO: 192 | | | (14%) | (10%) | (5%) |
| GSKSPIQYIDIGNYTVSC | SEQ ID | ORF8 protein | 3/22 (14%) | 12/22 | 4/22 | 3/22 |
| LPF | NO: 193 | | | (55%) | (19%) | (14%) |
| RSKQRRPQGLPNNT | SEQ ID | nucleocapsid | 1/22 (5%) | 5/22 | 0/22 | 1/22 |
| | NO: 194 | phosphoprotein | | (23%) | (0%) | (5%) |
| DLKFPRGQGVPINT | SEQ ID | nucleocapsid | 0/22 (0%) | 0/22 | 0/22 | 0/22 |
| | NO: 195 | phosphoprotein | | (0%) | (0%) | (0%) |
| NTNSSPDDQIGYYRR | SEQ ID | nucleocapsid | 0/22 (0%) | 0/22 | 0/22 | 0/22 |
| | NO: 196 | phosphoprotein | | (0%) | (0%) | (0%) |
| KDHIGTRNPANNAA | SEQ ID | nucleocapsid | 0/22 (0%) | 0/22 | 0/22 | 0/22 |
| | NO: 197 | phosphoprotein | | (0%) | (0%) | (0%) |
| RGTSPARMAGNGGDA | SEQ ID | nucleocapsid | 1/22 (5%) | 3/22 | 0/22 | 0/22 |
| | NO: 198 | phosphoprotein | | (14%) | (0%) | (0%) |
| MAGNGGDAALALLLLDR | SEQ ID | nucleocapsid | 0/22 (0%) | 2/22 | 2/22 | 1/22 |
| | NO: 199 | phosphoprotein | | (10%) | (10%) | (5%) |
| ALLLLDRLNQLESKMSG | SEQ ID | nucleocapsid | 3/22 (14%) | 5/22 | 2/22 | 5/22 |
| | NO: 200 | phosphoprotein | | (23%) | (10%) | (23%) |
| NQLESKMSGKGQQQQGQ | SEQ ID | nucleocapsid | 12/22 (55%) | 18/22 | 8/22 | 15/22 |
| TVTKKSA | NO: 201 | phosphoprotein | | (82%) | (37%) | (69%) |
| QTVTKKSAAEASKKPRQK | SEQ ID | nucleocapsid | 2/22 (10%) | 7/22 | 1/22 | 4/22 |
| | NO: 202 | phosphoprotein | | (32%) | (5%) | (19%) |
| RGPEQTQGNFGDQELI | SEQ ID | nucleocapsid | 6/22 (28%) | 3/22 | 2/22 | 3/22 |
| | NO: 203 | phosphoprotein | | (14%) | (10%) | (14%) |
| IKLDDKDPNFKDQVIL | SEQ ID | nucleocapsid | 4/22 (19%) | 1/22 | 0/22 | 0/22 |
| | NO: 204 | phosphoprotein | | (5%) | (0%) | (0%) |
| AYKTFPPTEPKKDKKKK | SEQ ID | nucleocapsid | 2/22 (10%) | 5/22 | 0/22 | 2/22 |
| | NO: 205 | phosphoprotein | | (23%) | (0%) | (10%) |
| KDKKKKADETQALPQR | SEQ ID | nucleocapsid | 3/22 (14%) | 7/22 | 0/22 | 5/22 |
| | NO: 206 | phosphoprotein | | (32%) | (0%) | (23%) |
| ADETQALPQRQKKQQTV | SEQ ID | nucleocapsid | 4/22 (19%) | 17/22 | 4/22 | 9/22 |
| TLLPAADL | NO: 207 | phosphoprotein | | (78%) | (19%) | (41%) |
| TLLPAADLDDFSKQLQQS | SEQ ID | nucleocapsid | 14/22 (64%) | 22/22 | 6/22 | 18/22 |
| MSSADS | NO: 208 | phosphoprotein | | (100%) | (28%) | (82%) |

| | | | | Healthy- | |
|---|---|---|---|---|---|
| | | | | Controls_ | SARS-CoV- |
| Epitope sequence | | Asymtomatic | Other_CoV | China | 1 (2003) |
| YRKVLLRKNGNKGA | SEQ ID | 0/12 (0%) | 0/12 (0%) | 0/10 (0%) | 0/10 (0%) |
| | NO: 46 | | | | |
| LLRKNGNKGAGGHSY | SEQ ID | 0/12 (0%) | 1/12 (9%) | 0/10 (0%) | 0/10 (0%) |
| | NO: 47 | | | | |
| GNKGAGGHSYGADLKSFD | SEQ ID | 0/12 (0%) | 2/12 (17%) | 0/10 (0%) | 0/10 (0%) |
| L | NO: 48 | | | | |
| DELGTDPYEDFQEN | SEQ ID | 0/12 (0%) | 0/12 (0%) | 1/10 (10%) | 0/10 (0%) |
| | NO: 49 | | | | |
| WNTKHSSGVTRELM | SEQ ID | 0/12 (0%) | 0/12 (0%) | 0/10 (0%) | 0/10 (0%) |
| | NO: 50 | | | | |
| GVYCCREHEHEIAW | SEQ ID | 0/12 (0%) | 0/12 (0%) | 1/10 (10%) | 0/10 (0%) |
| | NO: 51 | | | | |
| HEIAWYTERSEKSY | SEQ ID | 0/12 (0%) | 0/12 (0%) | 1/10 (10%) | 0/10 (0%) |
| | NO: 52 | | | | |
| SYELQTPFEIKLAK | SEQ ID | 0/12 (0%) | 0/12 (0%) | 0/10 (0%) | 0/10 (0%) |
| | NO: 53 | | | | |
| AKKFDTFNGECPNF | SEQ ID | 0/12 (0%) | 0/12 (0%) | 1/10 (10%) | 0/10 (0%) |
| | NO: 54 | | | | |
| CDHCGETSWQTGDF | SEQ ID | 0/12 (0%) | 0/12 (0%) | 0/10 (0%) | 0/10 (0%) |
| | NO: 55 | | | | |
| LTNIFGTVYEKLKPVLD | SEQ ID | 2/12 (17%) | 0/12 (0%) | 0/10 (0%) | 0/10 (0%) |
| | NO: 56 | | | | |
| GVEFLRDGWEIVKF | SEQ ID | 0/12 (0%) | 0/12 (0%) | 0/10 (0%) | 0/10 (0%) |
| | NO: 57 | | | | |
| TGDLQPLEQPTSEAVEAP | SEQ ID | 0/12 (0%) | 1/12 (9%) | 1/10 (10%) | 1/10 (10%) |
| LV | NO: 58 | | | | |
| KGGAPTKVTFGDDTVIEV | SEQ ID | 0/12 (0%) | 0/12 (0%) | 1/10 (10%) | 1/10 (10%) |
| | NO: 59 | | | | |
| NITFELDERIDKVL | SEQ ID | 0/12 (0%) | 0/12 (0%) | 1/10 (10%) | 0/10 (0%) |
| | NO: 60 | | | | |
| EFACVVADAVIKTLQPVS | SEQ ID | 0/12 (0%) | 0/12 (0%) | 0/10 (0%) | 0/10 (0%) |
| | NO: 61 | | | | |
| NKGEDIQLLKSAYENF | SEQ ID | 0/12 (0%) | 0/12 (0%) | 0/10 (0%) | 0/10 (0%) |
| | NO: 62 | | | | |
| YDKLVSSFLEMKSEK | SEQ ID | 0/12 (0%) | 0/12 (0%) | 0/10 (0%) | 0/10 (0%) |
| | NO: 63 | | | | |
| AFYILPSIISNEKQEI | SEQ ID | 0/12 (0%) | 1/12 (9%) | 1/10 (10%) | 0/10 (0%) |
| | NO: 64 | | | | |
| LGYVTHGLNLEEAA | SEQ ID | 0/12 (0%) | 0/12 (0%) | 0/10 (0%) | 0/10 (0%) |
| | NO: 65 | | | | |
| THGLNLEEAARYMRS | SEQ ID | 0/12 (0%) | 1/12 (9%) | 0/10 (0%) | 0/10 (0%) |
| | NO: 66 | | | | |
| TAYNGYLTSSSKTP | SEQ ID | 0/12 (0%) | 1/12 (9%) | 0/10 (0%) | 1/10 (10%) |
| | NO: 67 | | | | |
| GEVITFDNLKTLLS | SEQ ID | 0/12 (0%) | 0/12 (0%) | 0/10 (0%) | 0/10 (0%) |
| | NO: 68 | | | | |
| GRYMSALNHTKKWKYPQV | SEQ ID | 0/12 (0%) | 1/12 (9%) | 0/10 (0%) | 0/10 (0%) |
| | NO: 69 | | | | |
| TVGELGDVRETMSYL | SEQ ID | 0/12 (0%) | 0/12 (0%) | 0/10 (0%) | 0/10 (0%) |
| | NO: 70 | | | | |
| MGTLSYEQFKKGVQIPC | SEQ ID | 0/12 (0%) | 0/12 (0%) | 2/10 (20%) | 0/10 (0%) |
| | NO: 71 | | | | |
| FTCASEYTGNYQCGHY | SEQ ID | 0/12 (0%) | 0/12 (0%) | 0/10 (0%) | 0/10 (0%) |
| | NO: 72 | | | | |
| YQCGHYKHITSKET | SEQ ID | 0/12 (0%) | 0/12 (0%) | 0/10 (0%) | 1/10 (10%) |
| | NO: 73 | | | | |
| LVPNQPYPNASFDNF | SEQ ID | 0/12 (0%) | 0/12 (0%) | 0/10 (0%) | 0/10 (0%) |
| | NO: 74 | | | | |
| FPDLNGDVVAIDYK | SEQ ID | 0/12 (0%) | 0/12 (0%) | 0/10 (0%) | 0/10 (0%) |
| | NO: 75 | | | | |
| NSFDVLKSEDAQGMDNL | SEQ ID | 0/12 (0%) | 0/12 (0%) | 0/10 (0%) | 1/10 (10%) |
| | NO: 76 | | | | |
| SEEVVENPTIQKDVLECN | SEQ ID | 0/12 (0%) | 0/12 (0%) | 0/10 (0%) | 0/10 (0%) |
| | NO: 77 | | | | |
| GLAAVNSVPWDTIA | SEQ ID | 0/12 (0%) | 0/12 (0%) | 0/10 (0%) | 0/10 (0%) |
| | NO: 78 | | | | |
| TIANYAKPFLNKVVSTTT | SEQ ID | 0/12 (0%) | 3/12 (25%) | 2/10 (20%) | 0/10 (0%) |
| | NO: 79 | | | | |
| FLNKVVSTTTNIVTRC | SEQ ID | 0/12 (0%) | 1/12 (9%) | 0/10 (0%) | 0/10 (0%) |
| | NO: 80 | | | | |
| VKMFDAYVNTFSST | SEQ ID | 0/12 (0%) | 0/12 (0%) | 0/10 (0%) | 0/10 (0%) |
| | NO: 81 | | | | |
| FSSTFNVPMEKLKT | SEQ ID | 0/12 (0%) | 0/12 (0%) | 0/10 (0%) | 1/10 (10%) |
| | NO: 82 | | | | |
| TLVATAEAELAKNVSL | SEQ ID | 0/12 (0%) | 0/12 (0%) | 1/10 (10%) | 0/10 (0%) |
| | NO: 83 | | | | |
| SAAKKNNLPFKLTCA | SEQ ID | 0/12 (0%) | 0/12 (0%) | 0/10 (0%) | 0/10 (0%) |
| | NO: 84 | | | | |
| VHVMSKHTDFSSEIIG | SEQ ID | 0/12 (0%) | 0/12 (0%) | 0/10 (0%) | 0/10 (0%) |
| | NO: 85 | | | | |
| DFDTWFSQRGGSYT | SEQ ID | 0/12 (0%) | 0/12 (0%) | 1/10 (10%) | 0/10 (0%) |
| | NO: 86 | | | | |
| TFDSEYCRHGTCER | SEQ ID | 0/12 (0%) | 1/12 (9%) | 0/10 (0%) | 0/10 (0%) |
| | NO: 87 | | | | |
| DVLYQPPQTSITSAVL | SEQ ID | 1/12 (9%) | 0/12 (0%) | 0/10 (0%) | 0/10 (0%) |
| | NO: 88 | | | | |
| NRFTTTLNDFNLVAMK | SEQ ID | 0/12 (0%) | 0/12 (0%) | 0/10 (0%) | 0/10 (0%) |
| | NO: 89 | | | | |
| LTQDHVDILGPLSA | SEQ ID | 0/12 (0%) | 0/12 (0%) | 0/10 (0%) | 0/10 (0%) |
| | NO: 90 | | | | |
| GMNGRTILGSALLED | SEQ ID | 0/12 (0%) | 0/12 (0%) | 0/10 (0%) | 0/10 (0%) |
| | NO: 91 | | | | |
| QGLLPPKNSIDAFKLNIK | SEQ ID | 0/12 (0%) | 2/12 (17%) | 0/10 (0%) | 0/10 (0%) |
| | NO: 92 | | | | |
| KLKKSLNVAKSEFDRD | SEQ ID | 0/12 (0%) | 0/12 (0%) | 0/10 (0%) | 1/10 (10%) |
| | NO: 93 | | | | |
| LRANSAVKLQNNELS | SEQ ID | 0/12 (0%) | 0/12 (0%) | 1/10 (10%) | 0/10 (0%) |
| | NO: 94 | | | | |
| AVKLQNNELSPVALR | SEQ ID | 0/12 (0%) | 0/12 (0%) | 1/10 (10%) | 0/10 (0%) |
| | NO: 95 | | | | |
| CAAGTTQTACTDDN | SEQ ID | 0/12 (0%) | 0/12 (0%) | 0/10 (0%) | 0/10 (0%) |
| | NO: 96 | | | | |
| LALLSDLQDLKWARF | SEQ ID | 0/12 (0%) | 0/12 (0%) | 0/10 (0%) | 0/10 (0%) |
| | NO: 97 | | | | |
| ATVRLQAGNATEVPA | SEQ ID | 0/12 (0%) | 1/12 (9%) | 0/10 (0%) | 0/10 (0%) |
| | NO: 98 | | | | |
| VKMLCTHTGTGQAITV | SEQ ID | 0/12 (0%) | 0/12 (0%) | 0/10 (0%) | 0/10 (0%) |
| | NO: 99 | | | | |
| KYVQIPTTCANDPV | SEQ ID | 0/12 (0%) | 0/12 (0%) | 1/10 (10%) | 0/10 (0%) |
| | NO: 100 | | | | |
| RVCGVSAARLTPCGT | SEQ ID | 0/12 (0%) | 0/12 (0%) | 0/10 (0%) | 0/10 (0%) |
| | NO: 101 | | | | |
| AVAKHDFFKFRIDG | SEQ ID | 0/12 (0%) | 1/12 (9%) | 0/10 (0%) | 0/10 (0%) |
| | NO: 102 | | | | |
| GDFIQTTPGSGVPV | SEQ ID | 0/12 (0%) | 0/12 (0%) | 1/10 (10%) | 0/10 (0%) |
| | NO: 103 | | | | |
| QTYHPNCVNCLDDR | SEQ ID | 0/12 (0%) | 0/12 (0%) | 0/10 (0%) | 0/10 (0%) |
| | NO: 104 | | | | |
| ALTNNVAFQTVKPGNF | SEQ ID | 0/12 (0%) | 0/12 (0%) | 0/10 (0%) | 0/10 (0%) |
| | NO: 105 | | | | |
| GFFKEGSSVELKHF | SEQ ID | 0/12 (0%) | 0/12 (0%) | 0/10 (0%) | 0/10 (0%) |
| | NO: 106 | | | | |
| QLLFVVEVVDKYFDCYDG | SEQ ID | 0/12 (0%) | 3/12 (25%) | 1/10 (10%) | 0/10 (0%) |
| GC | NO: 107 | | | | |
| SLYVKPGGTSSGDATTAY | SEQ ID | 0/12 (0%) | 0/12 (0%) | 2/10 (20%) | 0/10 (0%) |
| A | NO: 108 | | | | |
| NRDVDTDFVNEFYAYL | SEQ ID | 0/12 (0%) | 0/12 (0%) | 0/10 (0%) | 0/10 (0%) |
| | NO: 109 | | | | |
| SQGLVASIKNFKSV | SEQ ID | 0/12 (0%) | 0/12 (0%) | 1/10 (10%) | 0/10 (0%) |
| | NO: 110 | | | | |
| QHTMLVKQGDDYVY | SEQ ID | 0/12 (0%) | 0/12 (0%) | 1/10 (10%) | 0/10 (0%) |
| | NO: 111 | | | | |
| VDDIVKTDGTLMIE | SEQ ID | 0/12 (0%) | 0/12 (0%) | 0/10 (0%) | 0/10 (0%) |
| | NO: 112 | | | | |
| AIDAYPLTKHPNQE | SEQ ID | 0/12 (0%) | 0/12 (0%) | 1/10 (10%) | 0/10 (0%) |
| | NO: 113 | | | | |
| GCDVTDVTQLYLGG | SEQ ID | 0/12 (0%) | 0/12 (0%) | 0/10 (0%) | 0/10 (0%) |
| | NO: 114 | | | | |
| GLYKNTCVGSDNVT | SEQ ID | 0/12 (0%) | 0/12 (0%) | 0/10 (0%) | 0/10 (0%) |
| | NO: 115 | | | | |
| NTCVGSDNVTDFNAIATC | SEQ ID | 0/12 (0%) | 0/12 (0%) | 0/10 (0%) | 0/10 (0%) |
| DW | NO: 116 | | | | |
| YQKVGMQKYSTLQG | SEQ ID | 0/12 (0%) | 0/12 (0%) | 0/10 (0%) | 1/10 (10%) |
| | NO: 117 | | | | |
| STLQGPPGTGKSHF | SEQ ID | 1/12 (9%) | 0/12 (0%) | 0/10 (0%) | 0/10 (0%) |
| | NO: 118 | | | | |
| ARARVECFDKFKVNS | SEQ ID | 0/12 (0%) | 1/12 (9%) | 0/10 (0%) | 0/10 (0%) |
| | NO: 119 | | | | |
| DPAQLPAPRTLLTK | SEQ ID | 0/12 (0%) | 0/12 (0%) | 0/10 (0%) | 1/10 (10%) |
| | NO: 120 | | | | |
| PRTLLTKGTLEPEY | SEQ ID | 0/12 (0%) | 0/12 (0%) | 0/10 (0%) | 0/10 (0%) |
| | NO: 121 | | | | |
| IFTQTTETAHSCNV | SEQ ID | 0/12 (0%) | 0/12 (0%) | 0/10 (0%) | 0/10 (0%) |
| | NO: 122 | | | | |
| GCHATREAVGTNLP | SEQ ID | 0/12 (0%) | 0/12 (0%) | 0/10 (0%) | 0/10 (0%) |
| | NO: 123 | | | | |
| VQMLSDTLKNLSDR | SEQ ID | 0/12 (0%) | 0/12 (0%) | 0/10 (0%) | 0/10 (0%) |
| | NO: 124 | | | | |
| FDYVYNPFMIDVQQWG | SEQ ID | 0/12 (0%) | 0/12 (0%) | 1/10 (10%) | 0/10 (0%) |
| | NO: 125 | | | | |
| MIDVQQWGFTGNLQ | SEQ ID | 0/12 (0%) | 0/12 (0%) | 0/10 (0%) | 0/10 (0%) |
| | NO: 126 | | | | |
| TGNLQSNHDLYCQVH | SEQ ID | 0/12 (0%) | 0/12 (0%) | 0/10 (0%) | 0/10 (0%) |
| | NO: 127 | | | | |
| LADKFPVLHDIGNPKAIK | SEQ ID | 0/12 (0%) | 1/12 (9%) | 0/10 (0%) | 1/10 (10%) |
| CVP | NO: 128 | | | | |
| FYSYATHSDKFTDGV | SEQ ID | 0/12 (0%) | 0/12 (0%) | 0/10 (0%) | 0/10 (0%) |
| | NO: 129 | | | | |
| FDTRVLSNLNLPGCD | SEQ ID | 0/12 (0%) | 1/12 (9%) | 0/10 (0%) | 0/10 (0%) |
| | NO: 130 | | | | |
| QVVSDIDYVPLKSATC | SEQ ID | 1/12 (9%) | 0/12 (0%) | 0/10 (0%) | 0/10 (0%) |
| | NO: 131 | | | | |
| PSVGPKQASLNGVTL | SEQ ID | 0/12 (0%) | 0/12 (0%) | 1/10 (10%) | 0/10 (0%) |
| | NO: 132 | | | | |
| GVVQQLPETYFTQSRN | SEQ ID | 0/12 (0%) | 0/12 (0%) | 0/10 (0%) | 0/10 (0%) |
| | NO: 133 | | | | |
| AMDEFIERYKLEGY | SEQ ID | 0/12 (0%) | 0/12 (0%) | 0/10 (0%) | 0/10 (0%) |
| | NO: 134 | | | | |
| LAKRFKESPFELED | SEQ ID | 0/12 (0%) | 0/12 (0%) | 1/10 (10%) | 1/10 (10%) |
| | NO: 135 | | | | |
| FITDAQTGSSKCVCSVID | SEQ ID | 0/12 (0%) | 0/12 (0%) | 0/10 (0%) | 1/10 (10%) |
| | NO: 136 | | | | |
| IKSQDLSVVSKVVKV | SEQ ID | 0/12 (0%) | 1/12 (9%) | 0/10 (0%) | 0/10 (0%) |
| | NO: 137 | | | | |
| YPKLQSSQAWQPGV | SEQ ID | 0/12 (0%) | 0/12 (0%) | 0/10 (0%) | 1/10 (10%) |
| | NO: 138 | | | | |
| QRMLLEKCDLQNYG | SEQ ID | 0/12 (0%) | 0/12 (0%) | 1/10 (10%) | 0/10 (0%) |
| | NO: 139 | | | | |
| HFGAGSDKGVAPGTA | SEQ ID | 0/12 (0%) | 0/12 (0%) | 0/10 (0%) | 1/10 (10%) |
| | NO: 140 | | | | |
| ITEHSWNADLYKLM | SEQ ID | 0/12 (0%) | 0/12 (0%) | 0/10 (0%) | 0/10 (0%) |
| | NO: 141 | | | | |
| LSSYSLFDMSKFPLKL | SEQ ID | 0/12 (0%) | 0/12 (0%) | 0/10 (0%) | 0/10 (0%) |
| | NO: 142 | | | | |
| SLLLCRMNSRNYIA | SEQ ID | 0/12 (0%) | 0/12 (0%) | 0/10 (0%) | 0/10 (0%) |
| | NO: 143 | | | | |
| QCVNLTTRTQLPPA | SEQ ID | 0/12 (0%) | 0/12 (0%) | 1/10 (10%) | 0/10 (0%) |
| | NO: 144 | | | | |
| TQLPPAYTNSFTRGVYYP | SEQ ID | 0/12 (0%) | 0/12 (0%) | 0/10 (0%) | 0/10 (0%) |
| D | NO: 145 | | | | |
| FHAIHVSGTNGTKRF | SEQ ID | 0/12 (0%) | 0/12 (0%) | 0/10 (0%) | 0/10 (0%) |
| | NO: 146 | | | | |
| NLVRDLPQGFSALEPLV | SEQ ID | 0/12 (0%) | 0/12 (0%) | 0/10 (0%) | 0/10 (0%) |
| | NO: 147 | | | | |
| LALHRSYLTPGDSSSGWT | SEQ ID | 0/12 (0%) | 0/12 (0%) | 0/10 (0%) | 0/10 (0%) |
| | NO: 148 | | | | |
| KYNENGTITDAVDCALDP | SEQ ID | 0/12 (0%) | 0/12 (0%) | 0/10 (0%) | 0/10 (0%) |
| LS | NO: 149 | | | | |
| IYQAGSTPCNGVEGFNCY | SEQ ID | 0/12 (0%) | 0/12 (0%) | 1/10 (10%) | 0/10 (0%) |
| | NO: 150 | | | | |
| VLSFELLHAPATVC | SEQ ID | 0/12 (0%) | 0/12 (0%) | 0/10 (0%) | 0/10 (0%) |
| | NO: 151 | | | | |
| LLHAPATVCGPKKST | SEQ ID | 0/12 (0%) | 0/12 (0%) | 0/10 (0%) | 0/10 (0%) |
| | NO: 152 | | | | |
| VKNKCVNFNFNGLT | SEQ ID | 2/12 (17%) | 0/12 (0%) | 0/10 (0%) | 0/10 (0%) |
| | NO: 153 | | | | |
| TESNKKFLPFQQFGRDIA | SEQ ID | 3/12 (25%) | 0/12 (0%) | 0/10 (0%) | 0/10 (0%) |
| D | NO: 154 | | | | |
| TDAVRDPQTLEILDITPC | SEQ ID | 2/12 (17%) | 0/12 (0%) | 0/10 (0%) | 0/10 (0%) |
| S | NO: 155 | | | | |
| QVAVLYQDVNCTEVPVAI | SEQ ID | 0/12 (0%) | 0/12 (0%) | 0/10 (0%) | 0/10 (0%) |
| HADQLTPTW | NO: 156 | | | | |
| IHADQLTPTWRVYSTGSN | SEQ ID | 1/12 (9%) | 0/12 (0%) | 0/10 (0%) | 0/10 (0%) |
| | NO: 157 | | | | |
| YSTGSNVFQTRAGCL | SEQ ID | 0/12 (0%) | 0/12 (0%) | 0/10 (0%) | 0/10 (0%) |
| | NO: 158 | | | | |
| CASYQTQTNSPRRARSV | SEQ ID | 2/12 (17%) | 0/12 (0%) | 0/10 (0%) | 0/10 (0%) |
| | NO: 159 | | | | |
| ARSVASQSIIAYTMSLGA | SEQ ID | 2/12 (17%) | 0/12 (0%) | 0/10 (0%) | 0/10 (0%) |
| ENSVA | NO: 160 | | | | |
| PVSMTKTSVDCTMY | SEQ ID | 0/12 (0%) | 0/12 (0%) | 1/10 (10%) | 0/10 (0%) |
| | NO: 161 | | | | |
| RALTGIAVEQDKNTQEVF | SEQ ID | 2/12 (17%) | 0/12 (0%) | 0/10 (0%) | 0/10 (0%) |
| AQV | NO: 162 | | | | |
| FAQVKQIYKTPPIKDFGG | SEQ ID | 2/12 (17%) | 0/12 (0%) | 0/10 (0%) | 0/10 (0%) |
| FNF | NO: 163 | | | | |
| NFSQILPDPSKPSKRS | SEQ ID | 2/12 (17%) | 0/12 (0%) | 0/10 (0%) | 0/10 (0%) |
| | NO: 164 | | | | |
| KRSFIEDLLFNKVT | SEQ ID | 1/12 (9%) | 0/12 (0%) | 0/10 (0%) | 0/10 (0%) |
| | NO: 165 | | | | |
| EDLLFNKVTLADAGF | SEQ ID | 0/12 (0%) | 0/12 (0%) | 0/10 (0%) | 0/10 (0%) |
| | NO: 166 | | | | |
| YGDCLGDIAARDLIC | SEQ ID | 0/12 (0%) | 0/12 (0%) | 0/10 (0%) | 0/10 (0%) |
| | NO: 167 | | | | |
| ICAQKFNGLTVLPPLL | SEQ ID | 0/12 (0%) | 0/12 (0%) | 1/10 (10%) | 0/10 (0%) |
| | NO: 168 | | | | |
| FGAGAALQIPFAMQM | SEQ ID | 0/12 (0%) | 0/12 (0%) | 0/10 (0%) | 1/10 (10%) |
| | NO: 169 | | | | |
| NGIGVTQNVLYENQK | SEQ ID | 0/12 (0%) | 0/12 (0%) | 0/10 (0%) | 0/10 (0%) |
| | NO: 170 | | | | |
| IGKIQDSLSSTASA | SEQ ID | 0/12 (0%) | 1/12 (9%) | 1/10 (10%) | 0/10 (0%) |
| | NO: 171 | | | | |
| VLNDILSRLDKVEAE | SEQ ID | 0/12 (0%) | 0/12 (0%) | 0/10 (0%) | 0/10 (0%) |
| | NO: 172 | | | | |
| LSRLDKVEAEVQID | SEQ ID | 0/12 (0%) | 0/12 (0%) | 1/10 (10%) | 0/10 (0%) |
| | NO: 173 | | | | |
| AICHDGKAHFPREGVF | SEQ ID | 2/12 (17%) | 0/12 (0%) | 0/10 (0%) | 0/10 (0%) |
| | NO: 174 | | | | |
| HWFVTQRNFYEPQIITT | SEQ ID | 2/12 (17%) | 0/12 (0%) | 0/10 (0%) | 0/10 (0%) |
| | NO: 175 | | | | |
| VIGIVNNTVYDPLQPEL | SEQ ID | 0/12 (0%) | 0/12 (0%) | 0/10 (0%) | 0/10 (0%) |
| | NO: 176 | | | | |
| TVYDPLQPELDSFK | SEQ ID | 0/12 (0%) | 0/12 (0%) | 0/10 (0%) | 0/10 (0%) |
| | NO: 177 | | | | |
| PELDSFKEELDKYFKNHT | SEQ ID | 8/12 (67%) | 1/12 (9%) | 0/10 (0%) | 1/10 (10%) |
| SPDVD | NO: 178 | | | | |
| SPDVDLGDISGINASVV | SEQ ID | 0/12 (0%) | 1/12 (9%) | 2/10 (20%) | 1/10 (10%) |
| | NO: 179 | | | | |
| EIDRLNEVAKNLNESL | SEQ ID | 1/12 (9%) | 1/12 (9%) | 0/10 (0%) | 0/10 (0%) |
| | NO: 180 | | | | |
| EVAKNLNESLIDLQ | SEQ ID | 0/12 (0%) | 0/12 (0%) | 0/10 (0%) | 0/10 (0%) |
| | NO: 181 | | | | |
| SCGSCCKFDEDDSE | SEQ ID | 0/12 (0%) | 1/12 (9%) | 0/10 (0%) | 0/10 (0%) |
| | NO: 182 | | | | |
| FDEDDSEPVLKGVKLH | SEQ ID | 0/12 (0%) | 0/12 (0%) | 0/10 (0%) | 0/10 (0%) |
| | NO: 183 | | | | |
| TIGTVTLKQGEIKDAT | SEQ ID | 0/12 (0%) | 0/12 (0%) | 1/10 (10%) | 0/10 (0%) |
| | NO: 184 | | | | |
| EHDYQIGGYTEKWESGVK | SEQ ID | 0/12 (0%) | 0/12 (0%) | 0/10 (0%) | 0/10 (0%) |
| DCVVL | NO: 185 | | | | |
| MADSNGTITVEELK | SEQ ID | 0/12 (0%) | 0/12 (0%) | 0/10 (0%) | 0/10 (0%) |
| | NO: 186 | | | | |
| RSMWSFNPETNILLNV | SEQ ID | 0/12 (0%) | 0/12 (0%) | 0/10 (0%) | 0/10 (0%) |
| | NO: 187 | | | | |
| CDIKDLPKEITVATS | SEQ ID | 0/12 (0%) | 0/12 (0%) | 0/10 (0%) | 0/10 (0%) |
| | NO: 188 | | | | |
| KLGASQRVAGDSGFA | SEQ ID | 0/12 (0%) | 0/12 (0%) | 0/10 (0%) | 0/10 (0%) |
| | NO: 189 | | | | |
| QRVAGDSGFAAYSRY | SEQ ID | 0/12 (0%) | 0/12 (0%) | 0/10 (0%) | 0/10 (0%) |
| | NO: 190 | | | | |
| SSGTYEGNSPFHPLA | SEQ ID | 0/12 (0%) | 0/12 (0%) | 0/10 (0%) | 0/10 (0%) |
| | NO: 191 | | | | |
| LIELCVDEAGSKSPIQYI | SEQ ID | 0/12 (0%) | 0/12 (0%) | 0/10 (0%) | 0/10 (0%) |
| D | NO: 192 | | | | |
| GSKSPIQYIDIGNYTVSC | SEQ ID | 0/12 (0%) | 1/12 (9%) | 0/10 (0%) | 0/10 (0%) |
| LPF | NO: 193 | | | | |
| RSKQRRPQGLPNNT | SEQ ID | 0/12 (0%) | 0/12 (0%) | 0/10 (0%) | 0/10 (0%) |
| | NO: 194 | | | | |
| DLKFPRGQGVPINT | SEQ ID | 0/12 (0%) | 0/12 (0%) | 1/10 (10%) | 0/10 (0%) |
| | NO: 195 | | | | |
| NTNSSPDDQIGYYRR | SEQ ID | 0/12 (0%) | 0/12 (0%) | 0/10 (0%) | 1/10 (10%) |
| | NO: 196 | | | | |
| KDHIGTRNPANNAA | SEQ ID | 0/12 (0%) | 0/12 (0%) | 1/10 (10%) | 0/10 (0%) |
| | NO: 197 | | | | |
| RGTSPARMAGNGGDA | SEQ ID | 0/12 (0%) | 0/12 (0%) | 0/10 (0%) | 0/10 (0%) |
| | NO: 198 | | | | |
| MAGNGGDAALALLLLDR | SEQ ID | 0/12 (0%) | 0/12 (0%) | 0/10 (0%) | 0/10 (0%) |
| | NO: 199 | | | | |
| ALLLLDRLNQLESKMSG | SEQ ID | 2/12 (17%) | 0/12 (0%) | 0/10 (0%) | 0/10 (0%) |
| | NO: 200 | | | | |
| NQLESKMSGKGQQQQGQT | SEQ ID | 7/12 (59%) | 0/12 (0%) | 0/10 (0%) | 0/10 (0%) |
| VTKKSA | NO: 201 | | | | |
| QTVTKKSAAEASKKPRQK | SEQ ID | 2/12 (17%) | 0/12 (0%) | 0/10 (0%) | 0/10 (0%) |
| | NO: 202 | | | | |
| RGPEQTQGNFGDQELI | SEQ ID | 2/12 (17%) | 0/12 (0%) | 0/10 (0%) | 0/10 (0%) |
| | NO: 203 | | | | |
| IKLDDKDPNFKDQVIL | SEQ ID | 0/12 (0%) | 0/12 (0%) | 0/10 (0%) | 0/10 (0%) |
| | NO: 204 | | | | |
| AYKTFPPTEPKKDKKKK | SEQ ID | 2/12 (17%) | 0/12 (0%) | 0/10 (0%) | 0/10 (0%) |
| | NO: 205 | | | | |
| KDKKKKADETQALPQR | SEQ ID | 3/12 (25%) | 0/12 (0%) | 0/10 (0%) | 0/10 (0%) |
| | NO: 206 | | | | |
| ADETQALPQRQKKQQTVT | SEQ ID | 2/12 (17%) | 0/12 (0%) | 0/10 (0%) | 0/10 (0%) |
| LLPAADL | NO: 207 | | | | |
| TLLPAADLDDFSKQLQQS | SEQ ID | 7/12 (59%) | 1/12 (9%) | 0/10 (0%) | 0/10 (0%) |
| MSSADS | NO: 208 | | | | |

TABLE 5
IgG linear IgG epitopes for detection of HKU-1, NL63, OC43, 229E, SARS-CoV-1.
Sequences (aa) are based on proteome of reference sequences from NCBI

| | | | | Controls | | | | | |
|---|---|---|---|---|---|---|---|---|---|
| | | | | | | Other | | | |
| | | | Epitope | Protein | SARS- | Human | | | |
| | Virus name | | sequences | name | CoV-1 | CoV | Healthy | | |
| 1. | Human | SEQ ID | MVNPSKGSSGLLIT | Nucleo- | 0/10 | 4/12 | 0/10 (0%) | | |
| | coronavirus | NO: 209 | KLT | capsid | (0%) | (34%) | | | |
| | HKU1 | | | phospho- | | | | | |
| | | | | protein | | | | | |
| 2. | Human | SEQ ID | VKQLPEQFDSLNLS | Nucleo- | 0/10 | 2/12 | 1/10 (10%) | | |
| | coronavirus | NO: 210 | AGTQ | capsid | (0%) | (17%) | | | |
| | HKU1 | | | phospho- | | | | | |
| | | | | protein | | | | | |
| 3. | Human | SEQ ID | SLLATLDDPYVEDS | Nucleo- | 1/10 | 2/12 | 0/10 (0%) | | |
| | coronavirus | NO: 211 | VA | capsid | (10%) | (17%) | | | |
| | HKU1 | | | phospho- | | | | | |
| | | | | protein | | | | | |
| 4. | Human | SEQ ID | MNKSFLPQFTSDQA | Membrane | 5/10 | 4/12 | 6/10 (60%) | | |
| | coronavirus | NO: 212 | V | glyco- | (50%) | (34%) | | | |
| | HKU1 | | | protein | | | | | |
| 5. | Human | SEQ ID | WLNNFNVPSPLNW | Spike | 0/10 | 2/12 | 0/10 (0%) | | |
| | coronavirus | NO: 213 | ERKI | glyco- | (0%) | (17%) | | | |
| | HKU1 | | | protein | | | | | |
| 6. | Human | SEQ ID | ISASYRFVTFEPFNV | Spike | 2/10 | 4/12 | 2/10 (20%) | | |
| | coronavirus | NO: 214 | S | glyco- | (20%) | (34%) | | | |
| | HKU1 | | | protein | | | | | |
| 7. | Human | SEQ ID | SRSFFEDLLFDKVK | Spike | 0/10 | 1/12 | 1/10 (10%) | | |
| | coronavirus | NO: 215 | LS | glyco- | (0%) | (9%) | | | |
| | HKU1 | | | protein | | | | | |
| 8. | Human | SEQ ID | VDKQVILVSNNKE | orf1ab | 0/10 | 1/12 | 1/10 (10%) | | |
| | coronavirus | NO: 216 | DFDIIQ | poly- | (0%) | (9%) | | | |
| | HKU1 | | | protein | | | | | |
| 9. | Human | SEQ ID | LLHSKVKVVPNLY | orf1ab | 1/10 | 3/12 | 2/10 (20%) | | |
| | coronavirus | NO: 217 | VVV | poly- | (10%) | (25%) | | | |
| | HKU1 | | | protein | | | | | |
| 10. | Human | SEQ ID | FDLVQYDFTDYKL | orf1ab | 1/10 | 1/12 | 0/10 (0%) | | |
| | coronavirus | NO: 218 | EL | poly- | (10%) | (9%) | | | |
| | HKU1 | | | protein | | | | | |
| 11. | Human | SEQ ID | PASQVIVNNYDKSA | orf1ab | 0/10 | 2/12 | 1/10 (10%) | | |
| | coronavirus | NO: 219 | GY | poly- | (0%) | (17%) | | | |
| | HKU1 | | | protein | | | | | |
| 12. | Human | SEQ ID | CTGSPYIDDFNKIAS | orf1ab | 0/10 | 1/12 | 0/10 (0%) | | |
| | coronavirus | NO: 220 | | poly- | (0%) | (9%) | | | |
| | HKU1 | | | protein | | | | | |
| 13. | Human | SEQ ID | CWETGKVKPPLNK | orf1ab | 1/10 | 1/12 | 2/10 (20%) | | |
| | coronavirus | NO: 221 | NYVFTG | poly- | (10%) | (9%) | | | |
| | HKU1 | | | protein | | | | | |
| 14. | Human | SEQ ID | AKVRVDCYDKFKI | orf1ab | 1/10 | 2/12 | 0/10 (0%) | | |
| | coronavirus | NO: 222 | NDTTCKY | poly- | (10%) | (17%) | | | |
| | HKU1 | | | protein | | | | | |
| 15. | Human | SEQ ID | ISPYNSQNYVAKRV | orf1ab | 0/10 | 1/12 | 1/10 (10%) | | |
| | coronavirus | NO: 223 | LGVQ | poly- | (0%) | (9%) | | | |
| | HKU1 | | | protein | | | | | |
| 16. | Human | SEQ ID | EFRDYFNNNTDSIV | orf1ab | 1/10 | 2/12 | 2/10 (20%) | | |
| | coronavirus | NO: 224 | | poly- | (10%) | (17%) | | | |
| | NL63 | | | protein | | | | | |
| 17. | Human | SEQ ID | RKASGLFDAIWDAF | orf1ab | 0/10 | 1/12 | 2/10 (20%) | | |
| | coronavirus | NO: 225 | VAAI | poly- | (0%) | (9%) | | | |
| | NL63 | | | protein | | | | | |
| 18. | Human | SEQ ID | NVGEPIISQPSKLLN | orf1ab | 2/10 | 1/12 | 0/10 (0%) | | |
| | coronavirus | NO: 226 | G | poly- | (20%) | (9%) | | | |
| | NL63 | | | protein | | | | | |
| 19. | Human | SEQ ID | ISYLNRARGSSAAR | orf1ab | 1/10 | 2/12 | 0/10 (0%) | | |
| | coronavirus | NO: 227 | LEPC | poly- | (10%) | (17%) | | | |
| | NL63 | | | protein | | | | | |
| 20. | Human | SEQ ID | CIKACEVVVTNLNK | orf1ab | 1/10 | 3/12 | 1/10 (10%) | | |
| | coronavirus | NO: 228 | SAGWPLNKFGKA | poly- | (10%) | (25%) | | | |
| | NL63 | | | protein | | | | | |
| 21. | Human | SEQ ID | VFINKYGLEDFNFE | orf1ab | 0/10 | 1/12 | 0/10 (0%) | | |
| | coronavirus | NO: 229 | H | poly- | (0%) | (9%) | | | |
| | NL63 | | | protein | | | | | |
| 22. | Human | SEQ ID | TVLNASIPESKPLA | Nucleo- | 7/10 | 3/12 | 6/10 (60%) | | |
| | coronavirus | NO: 230 | DDDSA | capsid | (70%) | (25%) | | | |
| | NL63 | | | phospho- | | | | | |
| | | | | protein | | | | | |
| 23. | Human | SEQ ID | ASQEGVLSEREKLL | Membrane | 0/10 | 2/12 | 0/10 (0%) | | |
| | coronavirus | NO: 231 | HLI | glyco- | (0%) | (17%) | | | |
| | NL63 | | | protein | | | | | |
| 24. | Human | SEQ ID | QFGLQDGFYSANFL | Spike | 3/10 | 4/12 | 6/10 (60%) | | |
| | coronavirus | NO: 232 | DDNVL | glyco- | (30%) | (34%) | | | |
| | NL63 | | | protein | | | | | |
| 25. | Human | SEQ ID | CDCGSTKLPYYEFE | Spike | 0/10 | 3/12 | 7/10 (70%) | | |
| | coronavirus | NO: 233 | KVHVQ | glyco- | (0%) | (25%) | | | |
| | NL63 | | | protein | | | | | |
| 26. | Human | SEQ ID | FDLVQYDFTDYKL | nsp9 | 1/10 | 1/12 | 0/10 (0%) | | |
| | coronavirus | NO: 234 | EL | | (10%) | (9%) | | | |
| | OC43 | | | | | | | | |
| 27. | Human | SEQ ID | SQVIVNNYDKSAG | nsp9 | 0/10 | 2/12 | 0/10 (0%) | | |
| | coronavirus | NO: 235 | YPF | | (0%) | (17%) | | | |
| | OC43 | | | | | | | | |
| 28. | Human | SEQ ID | LSTCDGQKFTDESF | nsp9 | 0/10 | 2/12 | 1/10 (10%) | | |
| | coronavirus | NO: 236 | YKNMY | | (0%) | (17%) | | | |
| | OC43 | | | | | | | | |
| 29. | Human | SEQ ID | FPLKLAGTAVINLR | nsp13 | 0/10 | 2/12 | 0/10 (0%) | | |
| | coronavirus | NO: 237 | AD | | (0%) | (17%) | | | |
| | OC43 | | | | | | | | |
| 30. | Human | SEQ ID | HATRDSIGTNFPLQ | nsp11 | 0/10 | 1/12 | 1/10 (10%) | | |
| | coronavirus | NO: 238 | L | | (0%) | (9%) | | | |
| | OC43 | | | | | | | | |
| 31. | Human | SEQ ID | CTGSPYIDDFNRIAS | nsp10 | 0/10 | 1/12 | 0/10 (0%) | | |
| | coronavirus | NO: 239 | | | (0%) | (9%) | | | |
| | OC43 | | | | | | | | |
| 32. | Human | SEQ ID | AKVRVECYDKFKI | nsp10 | 1/10 | 3/12 | 0/10 (0%) | | |
| | coronavirus | NO: 240 | NDTTRKYV | | (10%) | (25%) | | | |
| | OC43 | | | | | | | | |
| 33. | Human | SEQ ID | ASSRSAIEDLLFDK | Spike | 5/10 | 3/12 | 5/10 (50%) | | |
| | coronavirus | NO: 241 | VKLS | glyco- | (50%) | (25%) | | | |
| | OC43 | | | protein | | | | | |
| 34. | Human | SEQ ID | SQVIVNNYDKSAG | orf1ab | 0/10 | 2/12 | 0/10 (0%) | | |
| | coronavirus | NO: 242 | YPF | poly- | (0%) | (17%) | | | |
| | OC43 | | | protein | | | | | |
| 35. | Human | SEQ ID | LSTCDGQKFTDESF | orf1ab | 0/10 | 2/12 | 1/10 (10%) | | |
| | coronavirus | NO: 243 | YKNMY | poly- | (0%) | (17%) | | | |
| | OC43 | | | protein | | | | | |
| 36. | Human | SEQ ID | AETQKATEEAFKQS | orf1ab | 0/10 | 2/12 | 0/10 (0%) | | |
| | coronavirus | NO: 244 | YA | poly- | (0%) | (17%) | | | |
| | OC43 | | | protein | | | | | |
| 37. | Human | SEQ ID | SWEIGKVKPPLNKN | orf1ab | 1/10 | 1/12 | 3/10 (30%) | | |
| | coronavirus | NO: 245 | YVFTG | poly- | (10%) | (9%) | | | |
| | OC43 | | | protein | | | | | |
| 38. | Human | SEQ ID | AKVRVECYDKFKI | orf1ab | 2/10 | 3/12 | 1/10 (10%) | | |
| | coronavirus | NO: 246 | NDTTRKYV | poly- | (20%) | (25%) | | | |
| | OC43 | | | protein | | | | | |
| 39. | Human | SEQ ID | HATRDSIGTNFPLQ | orf1ab | 0/10 | 2/12 | 1/10 (10%) | | |
| | coronavirus | NO: 247 | L | poly- | (0%) | (17%) | | | |
| | OC43 | | | protein | | | | | |
| 40. | Human | SEQ ID | DVIDNEIIVKPNISL | orf1ab | 3/10 | 4/12 | 3/10 (30%) | | |
| | coronavirus | NO: 248 | CVPL | poly- | (30%) | (34%) | | | |
| | 229E | | | protein | | | | | |
| 41. | Human | SEQ ID | KHSDFGLGDLVDS | orf1ab | 0/10 | 1/12 | 2/10 (20%) | | |
| | coronavirus | NO: 249 | YFEND | poly- | (0%) | (9%) | | | |
| | 229E | | | protein | | | | | |
| 42. | Human | SEQ ID | CITSREVVVTNLNK | orf1ab | 0/10 | 3/12 | 0/10 (0%) | | |
| | coronavirus | NO: 250 | SA | poly- | (0%) | (25%) | | | |
| | 229E | | | protein | | | | | |
| 43. | Human | SEQ ID | DVIDNEIIVKPNISL | orf1ab | 3/10 | 4/12 | 3/10 (30%) | | |
| | coronavirus | NO: 251 | CVPL | poly- | (30%) | (34%) | | | |
| | 229E | | | protein | | | | | |
| 44. | Human | SEQ ID | KHSDFGLGDLVDS | orf1ab | 0/10 | 1/12 | 2/10 (20%) | | |
| | coronavirus | NO: 252 | YFEND | poly- | (0%) | (9%) | | | |
| | 229E | | | protein | | | | | |
| 45. | SARS-CoV-1 | SEQ ID | LYEQVVMDYLDNL | orf1ab | 7/10 | 3/12 | 4/10 (40%) | | |
| | | NO: 253 | KPRVEAPKQEEPPN | poly- | (70%) | (25%) | | | |
| | | | TED | protein | | | | | |
| 46. | SARS-CoV-1 | SEQ ID | VSVSSPDAVTTYNG | nsp3 | 3/10 | 0/12 | 2/10 (20%) | | |
| | | NO: 254 | YLTSSSKTSEEHF | | (30%) | (0%) | | | |
| 47. | SARS-CoV-1 | SEQ ID | LGDELGTDPIEDYE | orf1ab | 3/10 | 2/12 | 3/10 (30%) | | |
| | | NO: 255 | QNWNTKHGSGALR | poly- | (30%) | (17%) | | | |
| | | | | protein | | | | | |
| 48. | SARS-CoV-1 | SEQ ID | FTLRSITAQPVKIDN | Orf3 | 4/10 | 0/12 | 0/10 (0%) | | |
| | | NO: 256 | ASPASTVHATA | | (40%) | (0%) | | | |
| 49. | SARS-CoV-1 | SEQ ID | TIVVTEGDGISTPKL | Orf3 | 5/10 | 1/12 | 0/10 (0%) | | |
| | | NO: 257 | KEDYQIGGYSEDR | | (50%) | (9%) | | | |
| 50. | SARS-CoV-1 | SEQ ID | SSGVANPAMDPIYD | Orf3 | 4/10 | 1/12 | 1/10 (10%) | | |
| | | NO: 258 | EPTTTTSVPL | | (40%) | (9%) | | | |
| 51. | SARS-CoV-1 | SEQ ID | MADNGTITVEELK | membrane | 6/10 | 1/12 | 0/10 (0%) | | |
| | | NO: 259 | QLLEQWNLVIG | glyco- | (60%) | (9%) | | | |
| | | | | protein | | | | | |
| 52. | SARS-CoV-1 | SEQ ID | TPKDHIGTRNPNNN | Nucleo- | 5/10 | 2/12 | 0/10 (0%) | | |
| | | NO: 260 | AATVLQLPQGTTLP | capsid | (50%) | (17%) | | | |
| | | | KGFYAEGSRGGS | phospho- | | | | | |
| | | | | protein | | | | | |
| 53. | SARS-CoV-1 | SEQ ID | FSLDVSEKSGNFKH | spike | 4/10 | 0/12 | 1/10 (10%) | | |
| | | NO: 261 | LREFVF | glyco- | (40%) | (0%) | | | |
| | | | | protein | | | | | |
| | | | | | Asymp- | | Mild | | Severe |
| | | | | | to- | Mild | di- | Severe | di- |
| | | | | Asympto- | matic | di- | sease | di- | sease |
| | Epitope | | | matic | (Sec- | sease | (sec- | sease | (sec- |
| | sequences | | Protein name | (first) | ond) | (first) | ond) | (first) | ond) |
| 1. | MVNPSKGSSGLLIT | SEQ ID | Nucleocapsid | 0/6 (0%) | 0/6 | 0/22 | 1/22 | 0/22 | 0/22 |
| | KLT | NO: 209 | phosphoprotein | | (0%) | (0%) | (5%) | (0%) | (0%) |
| 2. | VKQLPEQFDSLNLS | SEQ ID | Nucleocapsid | 2/6 (34%) | 0/6 | 6/22 | 6/22 | 10/22 | 11/22 |
| | AGTQ | NO: 210 | phosphoprotein | | (0%) | (28%) | (28%) | (46%) | (50%) |
| 3. | SLLATLDDPYVEDS | SEQ ID | Nucleocapsid | 0/6 (0%) | 0/6 | 4/22 | 1/22 | 5/22 | 10/22 |
| | VA | NO: 211 | phosphoprotein | | (0%) | (19%) | (5%) | (23%) | (46%) |
| 4. | MNKSFLPQFTSDQA | SEQ ID | Membrane | 6/6 | 4/6 | 19/22 | 15/22 | 18/22 | 21/22 |
| | V | NO: 212 | glycoprotein | (100%) | (67%) | (87%) | (69%) | (82%) | (96%) |
| 5. | WLNNFNVPSPLNWE | SEQ ID | Spike | 0/6 (0%) | 0/6 | 0/22 | 0/22 | 1/22 | 0/22 |
| | RKI | NO: 213 | glycoprotein | | (0%) | (0%) | (0%) | (5%) | (0%) |
| 6. | ISASYRFVTFEPFNV | SEQ ID | Spike | 2/6 (34%) | 0/6 | 11/22 | 10/22 | 11/22 | 10/22 |
| | S | NO: 214 | glycoprotein | | (0%) | (50%) | (46%) | (50%) | (46%) |
| 7. | SRSFFEDLLFDKVKL | SEQ ID | Spike | 0/6 (0%) | 1/6 | 5/22 | 3/22 | 3/22 | 4/22 |
| | S | NO: 215 | glycoprotein | | (17%) | (23%) | (14%) | (14%) | (19%) |
| 8. | VDKQVILVSNNKED | SEQ ID | orf1ab | 0/6 (0%) | 0/6 | 0/22 | 2/22 | 7/22 | 11/22 |
| | FDIIQ | NO: 216 | polyprotein | | (0%) | (0%) | (10%) | (32%) | (50%) |
| 9. | LLHSKVKVVPNLYV | SEQ ID | orf1ab | 0/6 (0%) | 0/6 | 1/22 | 0/22 | 2/22 | 2/22 |
| | VV | NO: 217 | polyprotein | | (0%) | (5%) | (0%) | (10%) | (10%) |
| 10. | FDLVQYDFTDYKLE | SEQ ID | orf1ab | 0/6 (0%) | 0/6 | 4/22 | 2/22 | 7/22 | 11/22 |
| | L | NO: 218 | polyprotein | | (0%) | (19%) | (10%) | (32%) | (50%) |
| 11. | PASQVIVNNYDKSA | SEQ ID | orf1ab | 0/6 (0%) | 0/6 | 0/22 | 0/22 | 4/22 | 3/22 |
| | GY | NO: 219 | polyprotein | | (0%) | (0%) | (0%) | (19%) | (14%) |
| 12. | CTGSPYIDDFNKIAS | SEQ ID | orf1ab | 0/6 (0%) | 1/6 | 6/22 | 10/22 | 11/22 | 14/22 |
| | | NO: 220 | polyprotein | | (17%) | (28%) | (46%) | (50%) | (64%) |
| 13. | CWETGKVKPPLNK | SEQ ID | orf1ab | 1/6 (17%) | 2/6 | 4/22 | 5/22 | 4/22 | 5/22 |
| | NYVFTG | NO: 221 | polyprotein | | (34%) | (19%) | (23%) | (19%) | (23%) |
| 14. | AKVRVDCYDKFKIN | SEQ ID | orf1ab | 2/6 (34%) | 2/6 | 2/22 | 1/22 | 4/22 | 4/22 |
| | DTTCKY | NO: 222 | polyprotein | | (34%) | (10%) | (5%) | (19%) | (19%) |
| 15. | ISPYNSQNYVAKRV | SEQ ID | orf1ab | 0/6 (0%) | 0/6 | 2/22 | 2/22 | 5/22 | 6/22 |
| | LGVQ | NO: 223 | polyprotein | | (0%) | (10%) | (10%) | (23%) | (28%) |
| 16. | EFRDYFNNNTDSIV | SEQ ID | orf1ab | 0/6 (0%) | 0/6 | 3/22 | 3/22 | 6/22 | 8/22 |
| | | NO: 224 | polyprotein | | (0%) | (14%) | (14%) | (28%) | (37%) |
| 17. | RKASGLFDAIWDAF | SEQ ID | orf1ab | 0/6 (0%) | 0/6 | 2/22 | 1/22 | 0/22 | 1/22 |
| | VAAI | NO: 225 | polyprotein | | (0%) | (10%) | (5%) | (0%) | (5%) |
| 18. | NVGEPIISQPSKLLN | SEQ ID | orf1ab | 1/6 (17%) | 1/6 | 0/22 | 0/22 | 2/22 | 1/22 |
| | G | NO: 226 | polyprotein | | (17%) | (0%) | (0%) | (10%) | (5%) |
| 19. | ISYLNRARGSSAARL | SEQ ID | orf1ab | 0/6 (0%) | 0/6 | 0/22 | 0/22 | 0/22 | 0/22 |
| | EPC | NO: 227 | polyprotein | | (0%) | (0%) | (0%) | (0%) | (0%) |
| 20. | CIKACEVVVTNLNK | SEQ ID | orf1ab | 0/6 (0%) | 0/6 | 1/22 | 2/22 | 1/22 | 3/22 |
| | SAGWPLNKFGKA | NO: 228 | polyprotein | | (0%) | (5%) | (10%) | (5%) | (14%) |
| 21. | VFINKYGLEDFNFE | SEQ ID | orf1ab | 0/6 (0%) | 0/6 | 0/22 | 0/22 | 5/22 | 5/22 |
| | H | NO: 229 | polyprotein | | (0%) | (0%) | (0%) | (23%) | (23%) |
| 22. | TVLNASIPESKPLAD | SEQ ID | Nucleocapsid | 3/6 (50%) | 3/6 | 15/22 | 17/22 | 15/22 | 21/22 |
| | DDSA | NO: 230 | phosphoprotein | | (50%) | (69%) | (78%) | (69%) | (96%) |
| 23. | ASQEGVLSEREKLL | SEQ ID | Membrane | 0/6 (0%) | 0/6 | 0/22 | 1/22 | 1/22 | 2/22 |
| | HLI | NO: 231 | glycoprotein | | (0%) | (0%) | (5%) | (5%) | (10%) |
| 24. | QFGLQDGFYSANFL | SEQ ID | Spike | 1/6 (17%) | 1/6 | 6/22 | 7/22 | 8/22 | 13/22 |
| | DDNVL | NO: 232 | glycoprotein | | (17%) | (28%) | (32%) | (37%) | (60%) |
| 25. | CDCGSTKLPYYEFE | SEQ ID | Spike | 1/6 (17%) | 2/6 | 8/22 | 6/22 | 12/22 | 15/22 |
| | KVHVQ | NO: 233 | glycoprotein | | (34%) | (37%) | (28%) | (55%) | (69%) |
| 26. | FDLVQYDFTDYKLE | SEQ ID | nsp9 | 0/6 (0%) | 0/6 | 4/22 | 2/22 | 7/22 | 12/22 |
| | L | NO: 234 | | | (0%) | (19%) | (10%) | (32%) | (55%) |
| 27. | SQVIVNNYDKSAGY | SEQ ID | nsp9 | 0/6 (0%) | 0/6 | 0/22 | 0/22 | 6/22 | 9/22 |
| | PF | NO: 235 | | | (0%) | (0%) | (0%) | (28%) | (41%) |
| 28. | LSTCDGQKFTDESF | SEQ ID | nsp9 | 0/6 (0%) | 0/6 | 0/22 | 1/22 | 1/22 | 6/22 |
| | YKNMY | NO: 236 | | | (0%) | (0%) | (5%) | (5%) | (28%) |
| 29. | FPLKLAGTAVINLR | SEQ ID | nsp13 | 0/6 (0%) | 0/6 | 0/22 | 0/22 | 0/22 | 0/22 |
| | AD | NO: 237 | | | (0%) | (0%) | (0%) | (0%) | (0%) |
| 30. | HATRDSIGTNFPLQL | SEQ ID | nsp11 | 0/6 (0%) | 0/6 | 5/22 | 6/22 | 2/22 | 6/22 |
| | | NO: 238 | | | (0%) | (23%) | (28%) | (10%) | (28%) |
| 31. | CTGSPYIDDFNRIAS | SEQ ID | nsp10 | 0/6 (0%) | 0/6 | 2/22 | 2/22 | 3/22 | 5/22 |
| | | NO: 239 | | | (0%) | (10%) | (10%) | (14%) | (23%) |
| 32. | AKVRVECYDKFKIN | SEQ ID | nsp10 | 1/6 (17%) | 2/6 | 1/22 | 3/22 | 2/22 | 5/22 |
| | DTTRKYV | NO: 240 | | | (34%) | (5%) | (14%) | (10%) | (23%) |
| 33. | ASSRSAIEDLLFDKV | SEQ ID | Spike | 1/6 (17%) | 3/6 | 13/22 | 14/22 | 13/22 | 16/22 |
| | KLS | NO: 241 | glycoprotein | | (50%) | (60%) | (64%) | (60%) | (73%) |
| 34. | SQVIVNNYDKSAGY | SEQ ID | orf1ab | 0/6 (0%) | 0/6 | 0/22 | 0/22 | 0/22 | 0/22 |
| | PF | NO: 242 | polyprotein | | (0%) | (0%) | (0%) | (0%) | (0%) |
| 35. | LSTCDGQKFTDESF | SEQ ID | orf1ab | 0/6 (0%) | 0/6 | 0/22 | 1/22 | 1/22 | 6/22 |
| | YKNMY | NO: 243 | polyprotein | | (0%) | (0%) | (5%) | (5%) | (28%) |
| 36. | AETQKATEEAFKQS | SEQ ID | orf1ab | 0/6 (0%) | 0/6 | 0/22 | 0/22 | 0/22 | 0/22 |
| | YA | NO: 244 | polyprotein | | (0%) | (0%) | (0%) | (0%) | (0%) |
| 37. | SWEIGKVKPPLNKN | SEQ ID | orf1ab | 2/6 (34%) | 2/6 | 5/22 | 4/22 | 5/22 | 6/22 |
| | YVFTG | NO: 245 | polyprotein | | (34%) | (23%) | (19%) | (23%) | (28%) |
| 38. | AKVRVECYDKFKIN | SEQ ID | orf1ab | 1/6 (17%) | 2/6 | 1/22 | 3/22 | 5/22 | 8/22 |
| | DTTRKYV | NO: 246 | polyprotein | | (34%) | (5%) | (14%) | (23%) | (37%) |
| 39. | HATRDSIGTNFPLQL | SEQ ID | orf1ab | 0/6 (0%) | 0/6 | 6/22 | 7/22 | 2/22 | 6/22 |
| | | NO: 247 | polyprotein | | (0%) | (28%) | (32%) | (10%) | (28%) |
| 40. | DVIDNEIIVKPNISLC | SEQ ID | orf1ab | 1/6 (17%) | 0/6 | 9/22 | 11/22 | 16/22 | 19/22 |
| | VPL | NO: 248 | polyprotein | | (0%) | (41%) | (50%) | (73%) | (87%) |
| 41. | KHSDFGLGDLVDSY | SEQ ID | orf1ab | 0/6 (0%) | 1/6 | 10/22 | 7/22 | 15/22 | 19/22 |
| | FEND | NO: 249 | polyprotein | | (17%) | (46%) | (32%) | (69%) | (87%) |
| 42. | CITSREVVVTNLNKS | SEQ ID | orf1ab | 0/6 (0%) | 0/6 | 0/22 | 0/22 | 2/22 | 1/22 |
| | A | NO: 250 | polyprotein | | (0%) | (0%) | (0%) | (10%) | (5%) |
| 43. | DVIDNEIIVKPNISLC | SEQ ID | orf1ab | 1/6 (17%) | 0/6 | 10/22 | 11/22 | 16/22 | 19/22 |
| | VPL | NO: 251 | polyprotein | | (0%) | (46%) | (50%) | (73%) | (87%) |
| 44. | KHSDFGLGDLVDSY | SEQ ID | orf1ab | 0/6 (0%) | 0/6 | 12/22 | 7/22 | 15/22 | 19/22 |
| | FEND | NO: 252 | polyprotein | | (0%) | (55%) | (32%) | (69%) | (87%) |
| 45. | LYEQVVMDYLDNL | SEQ ID | orf1ab | 3/6 (50%) | 4/6 | 12/22 | 14/22 | 13/22 | 14/22 |
| | KPRVEAPKQEEPPN | NO: 253 | polyprotein | | (67%) | (55%) | (64%) | (60%) | (64%) |
| | TED | | | | | | | | |
| 46. | VSVSSPDAVTTYNG | SEQ ID | nsp3 | 0/6 (0%) | 0/6 | 10/22 | 10/22 | 7/22 | 12/22 |
| | YLTSSSKTSEEHF | NO: 254 | | | (0%) | (46%) | (46%) | (32%) | (55%) |
| 47. | LGDELGTDPIEDYE | SEQ ID | orf1ab | 0/6 (0%) | 0/6 | 10/22 | 13/22 | 17/22 | 18/22 |
| | QNWNTKHGSGALR | NO: 255 | polyprotein | | (0%) | (46%) | (60%) | (78%) | (82%) |
| 48. | FTLRSITAQPVKIDN | SEQ ID | Orf3 | 1/6 (17%) | 1/6 | 0/22 | 1/22 | 0/22 | 1/22 |
| | ASPASTVHATA | NO: 256 | | | (17%) | (0%) | (5%) | (0%) | (5%) |
| 49. | TIVVTEGDGISTPKL | SEQ ID | Orf3 | 0/6 (0%) | 0/6 | 0/22 | 0/22 | 2/22 | 1/22 |
| | KEDYQIGGYSEDR | NO: 257 | | | (0%) | (0%) | (0%) | (10%) | (5%) |
| 50. | SSGVANPAMDPIYD | SEQ ID | 0rf3 | 1/6 (17%) | 0/6 | 0/22 | 2/22 | 4/22 | 8/22 |
| | EPTTTTSVPL | NO: 258 | | | (0%) | (0%) | (10%) | (19%) | (37%) |
| 51. | MADNGTITVEELKQ | SEQ ID | membrane | 0/6 (0%) | 1/6 | 1/22 | 15/22 | 4/22 | 13/22 |
| | LLEQWNLVIG | NO: 259 | glycoprotein | | (17%) | (5%) | (69%) | (19%) | (60%) |
| 52. | TPKDHIGTRNPNNN | SEQ ID | Nucleocapsid | 2/6 (34%) | 3/6 | 10/22 | 16/22 | 15/22 | 22/22 |
| | AATVLQLPQGTTLP | NO: 260 | phosphoprotein | | (50%) | (46%) | (73%) | (69%) | (100%) |
| | KGFYAEGSRGGS | | | | | | | | |
| 53. | FSLDVSEKSGNFKH | SEQ ID | spike | 0/6 (0%) | 0/6 | 1/22 | 2/22 | 1/22 | 4/22 |
| | LREFVF | NO: 261 | glycoprotein | | (0%) | (5%) | (10%) | (5%) | (19%) |

### REFERENCES

Bolstad et al. A comparison of normalization methods for high density oligonucleotide array data based on avariance and bias. Bioinformatics 19, 185-193 (2003).
Buus et al. High-resolution mapping of linear antibody epitopes using ultrahigh-density peptide microarrays. Mol. Cell Proteomics 11, 1790-1800 (2012).
Centers for Disease Control & Prevention. Prevalence of IgG antibody to SARSassociated coronavirus in animal traders—Guangdong Province, China, 2003. MMWR Morb. Mortal Wkly Rep. 52, 986-987 (2003).
Chiaet al. Serological differentiation between COVID-19 and SARS infections. Emerg Microbes Infect. 9, 1497-1505 (2020).
Coronaviridae Study Group of the International Committee on Taxonomy of, V., The species Severe acute respiratory syndrome-related coronavirus: classifying 2019-nCoV and naming it SARS-CoV-2. Nat Microbiol, 2020. 5(4): p. 536-544.
Forsstrom et al. Proteome-wide epitope mapping of antibodies using ultradense peptide arrays. Mol. Cell Proteomics 13, 1585-1597 (2014).
Lv et al. Cross-reactive antibody response between SARS-CoV-2 and SARS-CoV Infections. Cell Rep. 31, 107725 (2020).
Lyamichev et al. Stepwise evolution improves identification of diverse peptides binding to a protein target. Sci Rep. 7, 12116 (2017).
Mishra et al. Antibodies to enteroviruses in cerebrospinal fluid of patients with acute flaccid myelitis. MBio 10, p.e01903-19 (2019).
Mishra et al. Diagnosis of zika virus infection by peptide array and enzyme-linked immunosorbent assay. MBio 9, e00095-18 (2018).
Poh et al. Two linear epitopes on the SARS-CoV-2 spike protein that elicit neutralising antibodies in COVID-19 patients. Nat. Commun. 11, 2806 (2020).
Tokarz et al. Identification of immunoreactive linear epitopes of Borrelia miyamotoi. Ticks Tick Borne Dis. 11, 101314 (2020).
Tokarz et al. A multiplex serologic platform for diagnosis of tick-borne diseases. Sci. Rep. 8, 3158 (2018).
Valentini et al. Peptide microarray-based characterization of antibody responses to host proteins after bacille Calmette-Guerin vaccination. Int. J. Infect. Dis. 56, 140-154 (2017).
Wu et al. Prolonged presence of SARS-CoV-2 viral RNA in faecal samples. Lancet Gastroenterol. Hepatol. 5, 434-435 (2020).
Xu et al. [Study on the dynamic prevalence of serum antibody against severe acute respiratory syndrome coronavirus in employees from wild animal market in Guangzhou]. Zhonghua Liu Xing Bing Xue Za Zhi 27, 950-952 (2006).
Yang et al. Linear epitopes of SARS-CoV-2 spike protein elicit neutralizing antibodies in COVID-19 patients. (2020).
Zhou et al. A pneumonia outbreak associated with a new coronavirus of probable bat origin. Nature, 2020. 579(7798): p. 270-273.

1. An isolated peptide that is strongly reactive with, and specific for antibodies to SARS-CoV-2, wherein the peptide comprises the amino acid sequence chosen from the group consisting of SEQ ID NOs: 1-45.

2. (canceled)

3. (canceled)

4. The collection of isolated peptides of claim 7, wherein the peptides range in length from about 6 amino acids to 50 amino acids.

5. The collection of isolated peptides of claim 7, wherein one or more of the isolated peptides is conjugated to a ligand, avidin, streptavidin, neutravidin, serum albumin, keyhole limpet hemocyanin (KLH), an enzyme, a metallic nanoparticle or nanoshell or is biotinylated.

6. The collection of isolated peptides of claim 7, wherein one or more of the isolated peptides is immobilized to a solid support.

7. A collection of isolated peptides that are strongly reactive with, and specific for antibodies to SARS-CoV-2, comprising: a peptide or peptide chosen from the group consisting of SEQ ID NOs: 1-45; and/or a collection or set of peptides shifted one residue across at least one peptide chosen from the group consisting of SEQ ID NOs: 1-45; or combinations thereof.

8. A peptide microarray comprising the collection of isolated peptides of claim 7.

9. A method for the differential serological detection of serological detection of exposure to and/or infection by SARS-CoV-2 in a sample, comprising:
a. contacting the sample with the collection of peptides of claim 7, under conditions sufficient to allow binding of antibody(ies) to the peptide(s); and
b. detecting formation of an antibody-peptide complex comprising said one or more peptides in the collection,
wherein formation of the complex is indicative of an antibody to an epitope of a SARS-CoV-2 antigen being present in the sample.

10. The method of claim 9, wherein the sample is from a subject.

11. The method of claim 10, wherein the subject is a test subject who has been administered a corona virus vaccine or immunomodulatory agent.

12. The method of claim 9, wherein the sample is chosen from the group consisting of nasopharyngeal aspirate, blood, cerebrospinal fluid, saliva, serum, plasma, urine, sputum, bronchial lavage, pericardial fluid, and peritoneal fluid.

13. The method of claim 9, wherein the sample is chosen from the group consisting of cells, cell culture, cell culture medium and compositions used for the development of pharmaceutical and therapeutic agents.

14. The method of claim 9, wherein the collection of peptides is immobilized to a solid support.

15. The method of claim 9, further comprising contacting the sample with a collection of peptides comprising a peptide or peptides chosen from the group consisting of SEQ ID NOs: 209-261; and/or a collection or set of peptides shifted one residue across at least one peptide chosen from the group consisting of SEQ ID NOs: 209-261; or combinations thereof, and further detecting the formation of an antibody-peptide complex comprising said one or more peptides in the collection, wherein formation of the complex is indicative of an antibody to an epitope of another human coronavirus other than SARS-CoV-2 being present in the sample

16. A method for the differential serological detection of exposure to and/or infection by SARS-CoV-2 in a sample, comprising:
a. contacting the sample with at least one peptide comprising the amino acid sequence chosen from the group consisting of SEQ ID NOs: 1-45, under conditions sufficient to allow binding of antibody(ies) in the sample to the peptide(s); and
b. detecting formation of an antibody-peptide complex comprising said one or more peptides,
wherein formation of the complex is indicative of an antibody to an epitope of SARS-CoV-2 being present in the sample.

17. The method of claim 16, wherein the sample is from a subject.

18. The method of claim 17, wherein the subject is a test subject which has been administered a SARS-CoV-2 vaccine or immunomodulatory agent.

19. The method of claim 16, wherein the sample is chosen from the group consisting of nasopharyngeal aspirate, blood, cerebrospinal fluid, saliva, serum, plasma, urine, sputum, bronchial lavage, pericardial fluid, and peritoneal fluid.

20. (canceled)

21. The method of claim 16, further comprising contacting the sample with at least one peptide comprising the amino acid sequence chosen from the group consisting of SEQ ID NOs: 209-261 and further detecting the formation of an antibody-peptide complex comprising said peptide(s), wherein formation of the complex is indicative of an antibody to an epitope of another human coronavirus other than SARS-CoV-2 being present in the sample

22. (canceled)

23. A kit comprising the collection of isolated peptides of claim 7.

24. (canceled)

25. The kit of claim 23, further comprising a collection of peptides comprising a peptide or peptides chosen from the group consisting of SEQ ID NOs: 209-261; and/or a collection or set of peptides shifted one residue across at least one peptide chosen from the group consisting of SEQ ID NOs: 209-261; or combinations thereof.
